(19) United States
(12) Patent Application Publication
(10) Pub. No.: US 2023/0414250 A1
(43) Pub. Date: Dec. 28, 2023

(54) PUNCTURE DEVICE GUIDE

(71) Applicant: CIVCO Medical Instruments Co., Inc., Kalona, IA (US)

(72) Inventor: Craig Joseph Cermak, Iowa City, IA (US)

(21) Appl. No.: 18/322,274

(22) Filed: May 23, 2023

Related U.S. Application Data
(60) Provisional application No. 63/366,968, filed on Jun. 24, 2022.

(51) Int. Cl. A61B 17/34; A61B 8/12; A61B 8/00

(52) U.S. Cl. CPC A61B 17/3403; A61B 8/12; A61B 8/4455; A61B 2010/045

(57) ABSTRACT

A puncture device guide comprises a guide body member configured to fixedly attach to an ultrasound probe and a lock assembly coupled to the guide body to fix the guide body relative to the ultrasound probe. The guide body member comprises a guide tube having a central aperture extending therethrough for receiving a puncture device; a forward securing member for engaging a forward portion of the ultrasound probe; and a rearward securing member for engaging a rearward portion of the ultrasound probe. The lock assembly is configured to be moveably coupled to the guide body member between a locked configuration and an unlocked configuration.

### CROSS-REFERENCE TO RELATED APPLICATIONS

This application claims priority under 35 U.S.C. § 119, based on U.S. Provisional Application No. 63/366968 filed Jun. 24, 2022, the disclosure of which is incorporated by reference herein.

### BACKGROUND

This invention relates to puncture device guidance devices for use with medical imaging instruments and more particularly to devices for guiding puncture devices to repeatable locations on a patient relative to a medical imaging instrument probe.

Imaging instruments, such as ultrasound probes, have revolutionized the manner in which many important medical procedures are performed. These medical instruments utilize imaging techniques to explore and assess the condition of human tissue and/or organs. As a result, diagnostic and therapeutic protocols have been developed that allow many highly successful and safe procedures to be performed with minimal disturbance to patients. For example, ultrasound probes have become an accepted modality for exploring endocavities, e.g., the digestive and reproductive tracts, of humans and animals in order to conduct routine examinations, as well as to identify evidence of tumors or other tissue regions of interest.

### BRIEF DESCRIPTION OF THE DRAWINGS

FIGS. 1A and 1B are top and left side views, respectively, illustrating one embodiment of a needle guidance device for use with an ultrasound probe, consistent with embodiments described herein;

FIGS. 1C and 1D are left side plan and rear isometric views, respectively, illustrating the needle guidance device of FIGS. 1A-1B, in an open or unlocked configuration;

FIGS. 1E-1G are left side, top, and cross-sectional views, respectively, of the guide body of FIGS. 1A-1D, consistent with implementations described herein;

FIGS. 1H-1K are left side, top, bottom, and exploded isometric views, respectively, of the lock assembly of FIGS. 1A-1D, consistent with implementations described herein;

FIGS. 2A and 2B are top and left side views, respectively, of another embodiment of a needle guidance device for use with an ultrasound probe, consistent with embodiments described herein;

FIGS. 2C and 2D are left side plan and rear isometric views, respectively, illustrating the needle guidance device of FIGS. 2A-2B, in an open or unlocked configuration;

FIGS. 2E-2G are left side, top, and cross-sectional views, respectively, of the guide body of FIGS. 2A-2D, consistent with implementations described herein;

FIGS. 2H-2K are left side, top, bottom, and exploded isometric views, respectively, of the lock assembly of FIGS. 2A-2D, consistent with implementations described herein;

FIGS. 3A and 3B are top and left side views, respectively, yet another embodiment of a needle guidance device for use with an ultrasound probe, consistent with embodiments described herein;

FIGS. 3C and 3D are left side plan and rear isometric views, respectively, illustrating the needle guidance device of FIGS. 3A-3B, in an open or unlocked configuration;

FIGS. 3E-3G are left side, top, and cross-sectional views, respectively, of the guide body of FIGS. 3A-3D, consistent with implementations described herein;

FIGS. 3H-3K are left side, top, bottom, and exploded isometric views, respectively, of the lock assembly of FIGS. 3A-3D, consistent with implementations described herein;

FIGS. 4A and 4B are top and left side views, respectively, of still another embodiment of a needle guidance device for use with an ultrasound probe, consistent with embodiments described herein;

FIGS. 4C and 4D are left side plan and rear isometric views, respectively, illustrating the needle guidance device of FIGS. 4A-4B, in an open or unlocked configuration;

FIGS. 4E-4G are left side, top, and cross-sectional views, respectively, of the guide body of FIGS. 4A-4D, consistent with implementations described herein;

FIGS. 4H-4K are left side, top, bottom, and exploded isometric views, respectively, of the lock assembly of FIGS. 4A-4D, consistent with implementations described herein;

FIGS. 5A and 5B are top and left side views, respectively, illustrating another embodiment of a needle guidance device for use with an ultrasound probe, consistent with embodiments described herein;

FIGS. 5C and 5D are left side plan and rear isometric views, respectively, illustrating the needle guidance device of FIGS. 5A-5B, in an open or unlocked configuration;

FIGS. 5E-5G are left side, top, and cross-sectional views, respectively, of the guide body of FIGS. 5A-5D, consistent with implementations described herein;

FIGS. 5H-5K are left side, top, bottom, and exploded isometric views, respectively, of the lock assembly of FIGS. 5A-5D, consistent with implementations described herein;

FIGS. 6A and 6B are top and left side views, respectively, another embodiment of a needle guidance device for use with an ultrasound probe, consistent with embodiments described herein;

FIGS. 6C and 6D are left side plan and rear isometric views, respectively, illustrating the needle guidance device of FIGS. 6A-6B, in an open or unlocked configuration;

FIGS. 6E-6G are left side, top, and cross-sectional views, respectively, of the guide body of FIGS. 6A-6D, consistent with implementations described herein;

FIGS. 6H-6K are left side, top, bottom, and exploded isometric views, respectively, of the lock assembly of FIGS. 6A-6D, consistent with implementations described herein;

FIGS. 7A and 7B are top and left side views, respectively, illustrating yet another embodiment of a needle guidance device for use with an ultrasound probe, consistent with embodiments described herein;

FIGS. 7C and 7D are left side plan and rear isometric views, respectively, illustrating the needle guidance device of FIGS. 7A-7B, in an open or unlocked configuration;

FIGS. 7E-7G are left side, top, and cross-sectional views, respectively, of the guide body of FIGS. 7A-7D, consistent with implementations described herein;

FIGS. 7H-7K are left side, top, bottom, and exploded isometric views, respectively, of the lock assembly of FIGS. 7A-7D, consistent with implementations described herein;

FIGS. 8A and 8B are top and left side views, respectively, another embodiment of a needle guidance device for use with an ultrasound probe, consistent with embodiments described herein;

FIGS. 8C and 8D are left side plan and rear isometric views, respectively, illustrating the needle guidance device of FIGS. 8A-8B, in an open or unlocked configuration;

FIGS. 8E-8G are left side, top, and cross-sectional views, respectively, of the guide body of FIGS. 8A-8D, consistent with implementations described herein;

FIGS. 8H-8K are left side, top, bottom, and exploded isometric views, respectively, of the lock assembly of FIGS. 8A-8D, consistent with implementations described herein;

FIGS. 9A and 9B are top and left side views, respectively, illustrating one embodiment of a needle guidance device for use with an ultrasound probe, consistent with embodiments described herein;

FIGS. 9C and 9D are front and rear isometric views, respectively, illustrating the needle guidance device of FIGS. 9A-9B, in an open or unlocked configuration;

FIGS. 9E-9G are rear and front isometric and top side views, respectively, of the guide body of FIGS. 9A-9D, consistent with implementations described herein;

FIGS. 9H-9K are left side, top, bottom, and exploded isometric views, respectively, of the lock assembly of FIGS. 9A-9D, consistent with implementations described herein;

FIGS. 10A and 10B are top and left side views, respectively, illustrating still another embodiment of a needle guidance device for use with an ultrasound probe, consistent with embodiments described herein;

FIGS. 10C and 10D are front and rear isometric views, respectively, illustrating the needle guidance device of FIGS. 10A-10B, in an open or unlocked configuration;

FIGS. 10E-10G are rear and front isometric and top side views, respectively, of the guide body of FIGS. 10A-10D, consistent with implementations described herein;

FIGS. 10H-10K are left side, top, bottom, and exploded isometric views, respectively, of the lock assembly of FIGS. 10A-10D, consistent with implementations described herein;

FIGS. 11A and 11B are top and left side views, respectively, illustrating another embodiment of a needle guidance device for use with an ultrasound probe, consistent with embodiments described herein;

FIGS. 11C and 11D are front and rear isometric views, respectively, illustrating the needle guidance device of FIGS. 11A-11B, in an open or unlocked configuration;

FIGS. 11E-11H are left side, top, front isometric, and front views, respectively, of the guide body of FIGS. 11A-11D, consistent with implementations described herein;

FIGS. 11I-11L are top, left side, bottom, and exploded isometric views, respectively, of the lock assembly of FIGS. 11A-11D, consistent with implementations described herein;

FIGS. 12A and 12B are top and left side views, respectively, illustrating still another embodiment of a needle guidance device for use with an ultrasound probe, consistent with embodiments described herein;

FIGS. 12C and 12D are front and rear isometric views, respectively, illustrating the needle guidance device of FIGS. 12A-12B, in an open or unlocked configuration;

FIGS. 12E-12H are left side, top, bottom, and front views, respectively, of the guide body of FIGS. 12A-12D, consistent with implementations described herein; and

FIGS. 12I-12L are left side, top, bottom, and exploded isometric views, respectively, of the lock assembly of FIGS. 12A-12D, consistent with implementations described herein;

### DETAILED DESCRIPTION OF THE PREFERRED EMBODIMENTS

The following detailed description refers to the accompanying drawings. The same reference numbers in different drawings may identify the same or similar elements. Also, the following detailed description does not limit the invention.

Implementations described herein relate to guidance devices for facilitating the placement of a puncture device (e.g., a needle) at a defined position relative to an ultrasound probe. More specifically, the guidance devices described below include components configured to be releasably secured to an ultrasound probe in a defined configuration relative to the ultrasound probe.

For example, in one implementation, the ultrasound probe may be an endocavity ultrasound probe and the guidance device may be configured to facilitate guidance of a needle, such as a biopsy needle at a predefined orientation or angle with respect to the ultrasound probe. Consistent with embodiments described herein, the needle guidance device may be releasably locked onto the ultrasound probe prior to use.

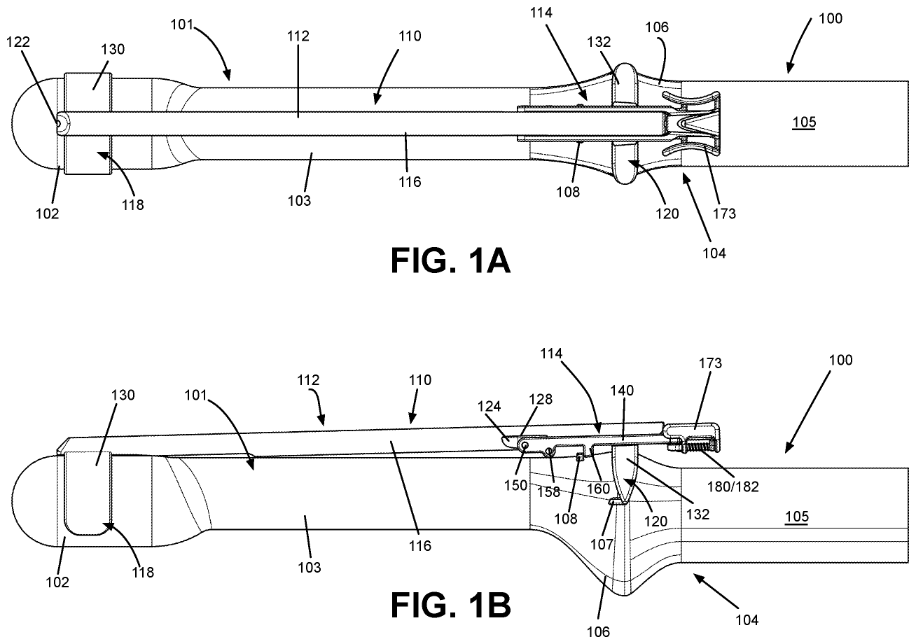

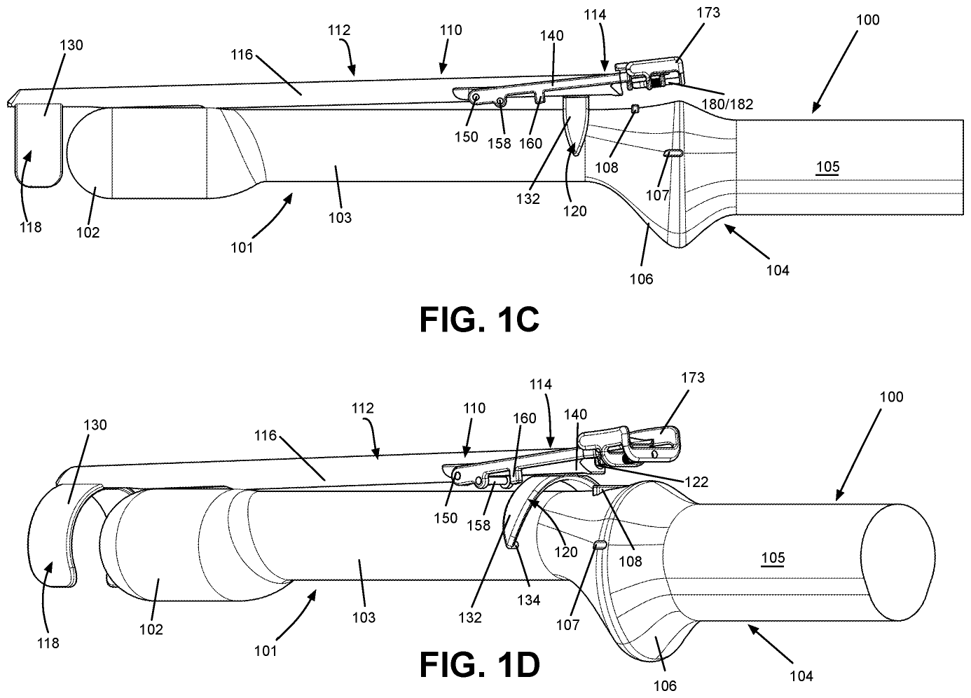

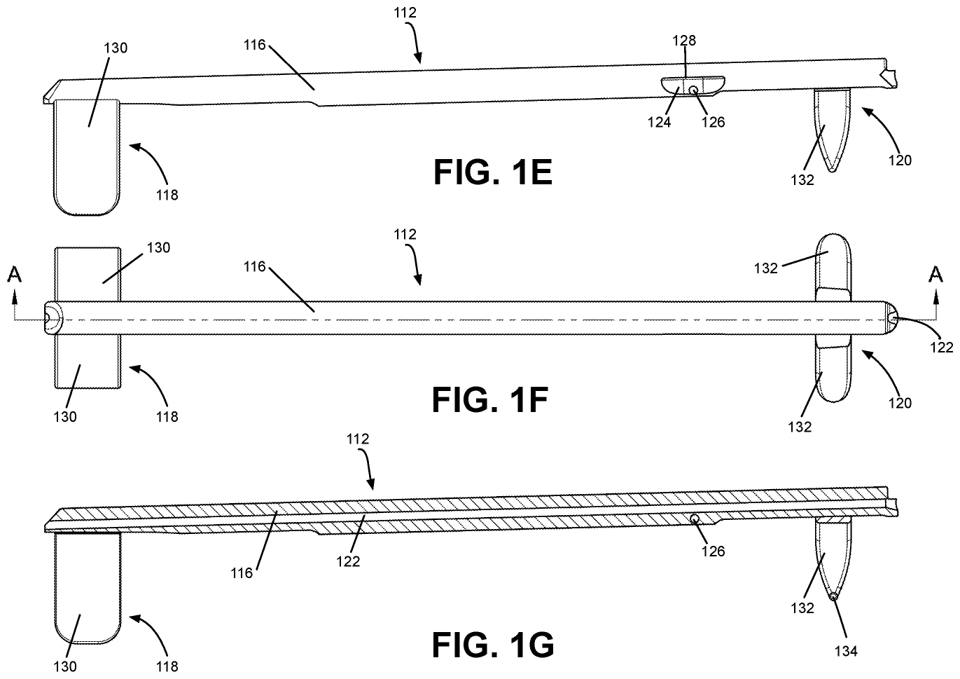

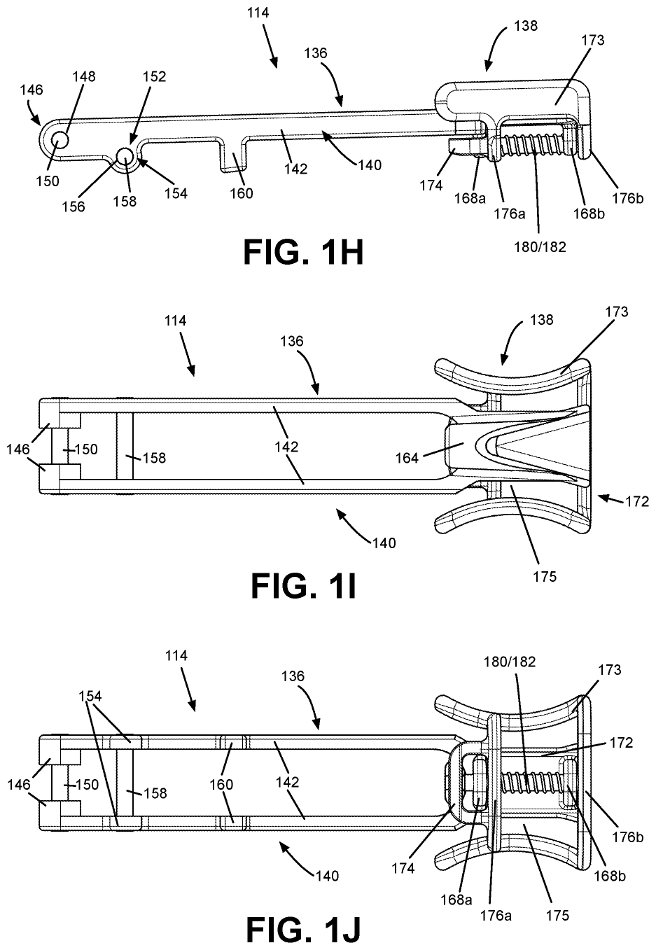

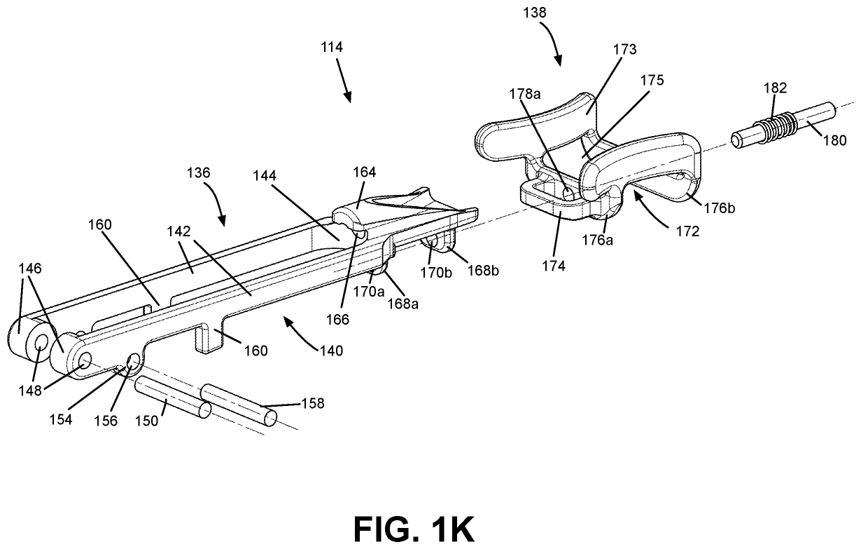

FIGS. 1A and 1B are top and left side views, respectively, illustrating one embodiment of a needle guidance device 110 for use with an ultrasound probe 100 in an assembled or locked configuration, consistent with embodiments described herein. FIG. 1C and 1D are left side plan and right rear isometric views, respectively, illustrating needle guidance device 110 and ultrasound probe 100 in an open or pre-assembled configuration.

Consistent with embodiments described herein, ultrasound probe 100 and needle guidance device 110 are configured to be lockingly coupled to each other so that, during use, needle guidance device 110 is fixed relative to ultrasound probe 100. As shown in FIGS. 1A-1D ultrasound probe 100 includes one or more features that facilitate cooperative engagement with needle guidance device 110. In particular, ultrasound probe 100 includes a generally tubular body 101 having a forward end 102, an intermediate portion 103, and a rearward end 104. As shown, forward end 102 includes a generally bulbous portion having a diameter larger than or similar size to that of intermediate portion 103. Rearward end 104 includes a handle portion 105 and a transition portion 106 to transition a shape of body 101 between handle portion 105 and intermediate portion 103. As shown, transition portion 106 includes a least a portion having a larger diameter than that of intermediate portion 103 and handle portion 105. The intermediate portion can be similar in size to the handle portion.

Consistent with implementations described herein, transition portion 106 includes a pair of locating pockets 107 positioned on opposite sides of probe 100 and a locating rib 108 projecting from a top surface of probe 100. Locating pockets 107 include a forward-facing cavity having a rear wall therein, which effectively act as stops for preventing further axial advancement of ultrasound probe 100 relative to needle guidance device 110. As described in additional detail below, locating pockets 107 and locating rib 108 collectively facilitate positioning and securing of needle guidance device 110 to ultrasound probe 100 during assembly.

Needle guidance device 110 includes a guide body 112 and a lock assembly 114. Guide body 112 includes a guide tube 116, a forward securing member 118, and a rear securing member 120. FIGS. 1E-1G are left side, top, and cross-sectional views, respectively, of guide body 112. The cross-sectional view of FIG. 1G is taken along the line A-A in FIG. 1F.

As shown in FIGS. 1E-1G, guide tube 116 comprises a generally cylindrical configuration having a central aperture 122 extending therethrough and sized for receiving a needle or other puncture device. In some implementations, guide tubes 116 having different sized apertures 122 may be provided for different gauge puncture devices. In some embodiments, rather than an enclosed central aperture 122, guide tube 116 may include a channel or slot formed vertically therein, into which the puncture device is received.

An intermediate portion of guide tube 116 includes cut out portion 124 that includes a transverse aperture 126 extending therethrough and positioned below the central aperture 122 in guide tube 116, as shown in FIG. 1G. As described below, transverse aperture 126 is sized to receive a pivot pin when assembled together with lock assembly 114. In addition, cut out portion 124 includes a planar upper portion 128, which forms a stop for engaging a portion of lock assembly 114, as also described below.

Forward securing member 118 includes opposing curved arms 130 that project downwardly from a forward portion of guide tube 116 and which include a shape configured to correspond to a shape of forward end 102 of ultrasound probe 100. During assembly, curved arms 130 are configured to engage forward end 102 of ultrasound probe 100 to positively center guide tube 116 longitudinally with respect to ultrasound probe 100. Consistent with the embodiment of FIGS. 1A-G, curved arms 130 may be configured to extend more than 180°. In this manner, insertion of forward end 102 of ultrasound probe 100 within curved arms 130 effectively captures an axial position of the forward end of guide tube 116 relative to ultrasound probe 100. That is, the forward end of guide tube 116 may not be pulled radially outwardly from ultrasound probe 100 when forward end 102 of ultrasound probe 100 is positioned with curved arms 130.

Rear securing member 120 also includes curved arms 132 that project downwardly from guide tube 116 and which are configured to engage an outer surface of transition portion 106 of ultrasound probe 100. Consistent with some implementations described herein, unlike curved arms 130 in forward securing member 118, curved arms 132 in rear securing member 120 include locating elements 134 which project inwardly at a terminus of curved arms 132. As shown in FIG. 1A, when assembled, locating elements 134 are slidingly positioned within locating pockets 107 in transition portion 106 of ultrasound probe 100.

FIGS. 1H-1K are left side, top, bottom, and exploded isometric views, respectively, of lock assembly 114 consistent with implementations described herein. As shown, lock assembly 114 includes a lever component 136 and a latch component 138 which together cooperatively engage guide tube 116 and ultrasound probe 100 to secure needle guidance device 100 to ultrasound probe 100 in the manner described below.

As shown in FIGS. 1I-1K, lever component 136 includes a body portion 140 having a pair of opposing arms 142 and a guide tube alignment portion 144. Arms 142 are sized to accommodate at least a portion of guide tube 116 therebetween, as shown in FIG. 1F. A forward end of each of arms 142 includes an engagement portion 146 for engaging cut out portion 124 in guide tube 116. As shown in FIGS. 1H and 1K, each engagement portion 146 includes an aperture 148 therethrough for aligning with transverse aperture 126 in cut out portion 124. During assembly, apertures 148 and 126 receive a pivot pin 150 therethrough.

Consistent with embodiments described herein, arms 142 also include a pivot limiting element 152 configured to limit the extent to which lever component 136 may be pivoted with respect to guide tube 116. In one implementation, pivot limiting element 152 includes downward projections 154 in arms 142 that include apertures 156 therethrough which may receive a limiting pin 158 therethrough. The position of apertures 156 relative to apertures 148 define the pivot limit for lock assembly 114. In other implementations, pivot limiting element 152 may include a different structure, such as a fixed bar which joins arms 142 in a location similar to limiting pin 158.

As shown in FIGS. 1H and 1K, arms 142 in lever component 136 may also include rib engagement elements 160 which project downwardly therefrom. As shown in FIG. 1A, when locked onto ultrasound probe 100, rib engagement elements 160 engage a rearward side of locating rib 108. When rib engagement elements 160 engage locating rib 108 and locating elements 134 in rear securing member 120 engage locating pockets 107, needle guidance device becomes fixed relative to ultrasound probe 100.

As shown, a rearward end of lever component 136 includes guide tube alignment portion 144 which joins arms 142. As shown in FIG. 1K, guide tube alignment portion 144 includes an upper guide tube engagement feature 164 and a central aperture 166 longitudinally positioned to align with central aperture 122 in guide tube 116, when latch component 138 is engaged with lever component 136 and guide tube 116, as described below. As shown in FIG. 1B, upper guide tube engagement feature 164 includes a portion which projects forwardly relative to a remainder of guide tube alignment portion 144. When lock assembly 114 is latched onto guide tube 116, upper guide tube engagement feature 164 is configured to engage an upper, rear end of guide tube 116 in a position in which aperture 166 is aligned with aperture 122.

As shown in FIGS. 1B, 1H, and 1K, guide tube alignment portion 144 further includes downwardly projecting tabs 168a and 168b. Tabs 168a and 168b include apertures 170a and 170b formed therethrough and function to engage a biasing element in latch component 138, as described below.

As shown in FIGS. 1I-1K and FIG. 1A-1B, latch component 138 comprises a generally frame-like member 172 configured to engage both a rearward portion of guide tube 116 of and lever component 136. In particular, as shown, latch component 138 includes a latch engagement portion 173 and a lower guide tube engagement feature 174. Latch engagement portion 173 projects upwardly from frame-like member 172 and provides an engagement surface with which a user may manipulate (i.e., open and close) latch component 138, as described below. Frame-like member 172 include cavity 175 therein for receiving at least a portion of guide tube alignment portion 144, as shown in FIG. 1A. Frame-like member 172 includes a pair of tab engagement elements 176a and 176b that include alignment apertures 178a and 178b formed therethrough. Lower guide tube engagement feature 174 projects forwardly from forward tab engagement element 176a and engages a lower surface of guide tube 116 when latch component 138 is in a latched position with respect to guide tube 116.

During assembly, guide tube alignment portion 144 of lever component 136 is inserted with cavity 175 such that tab engagement elements 176a and 176b abut respective rearward surfaces of tabs 168a and 168b. A rod 180 having a biasing element, such as a spring, 182 positioned thereon is inserted and secured within apertures 170a/170b and 178a/178b, such that biasing element 182 is positioned between a rearward side of forward tab engagement element 176a and a forward side of rearward tab 168b, as shown in FIG. 1J.

To secure needle guidance device 110 to ultrasound probe 100, an operator positions guide tube 116 relative to ultrasound probe 100 such that forward securing member 118 and rearward securing member of guide tube 116 engage forward end 102 and transition portion 106 of ultrasound probe 100, respectively. Guide tube 116 is then fixed relative to ultrasound probe 100 by seating locating elements 134 within locating pockets 107.

Latch component 138 is then opened by pulling rearwardly on latch engagement portion 173, thus compressing biasing element 182 and allowing lower guide tube engagement feature 174 to move rearwardly relative to lever component 136. Lever component 136 is then pivoted about pivot pin 150 such that rib engagement elements 168 engage a rearward side of locating rib 108. Latch engagement portion 173 is then released, which causes biasing element 182 to urge lower guide tube engagement feature 174 to move forwardly relative to lever component 136. In its forward position, lower guide tube engagement feature 174 engages a lower surface of guide tube 116 and upper guide tube engagement feature 164 engages an upper surface of guide tube 116, effectively securing guide tube 116 to ultrasound probe 100.

Consistent with embodiments described herein, central aperture 166 in guide tube alignment portion 144 is only aligned with central aperture 122 in guide tube 116 when guide tube 116 is securely latched onto ultrasound probe, thus preventing premature or unsafe use of needle guidance device 110.

Upon completion of a procedure, needle guidance device 110 may be removed from ultrasound probe 100 by reversing the above steps. That is, latch engagement portion 173 may be again moved rearwardly to release guide tube 116 from lower guide tube engagement feature 174. Lever component 136 may then be pivoted about pivot pin 150 such that rib engagement elements 168 becomes disengaged from locating rib 108. At this point, needle guidance device 110 may be removed from ultrasound probe 100 by removing locating elements 134 from locating pockets 107.

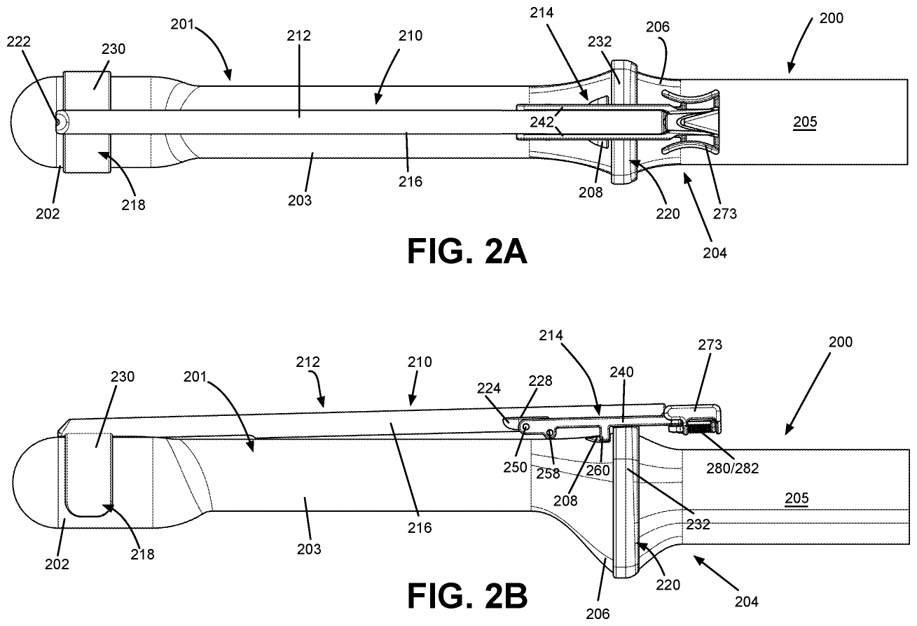

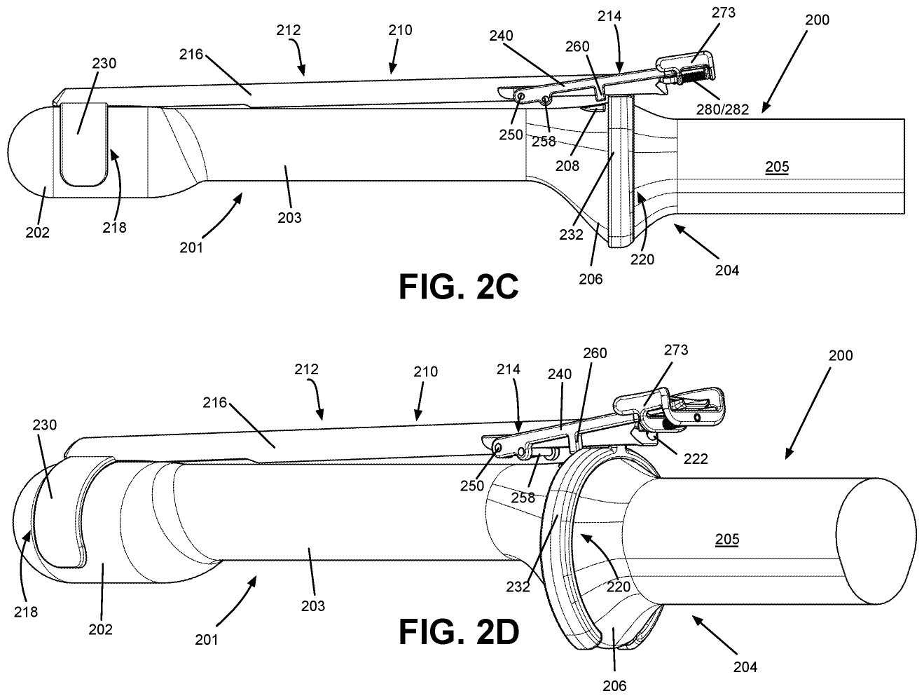

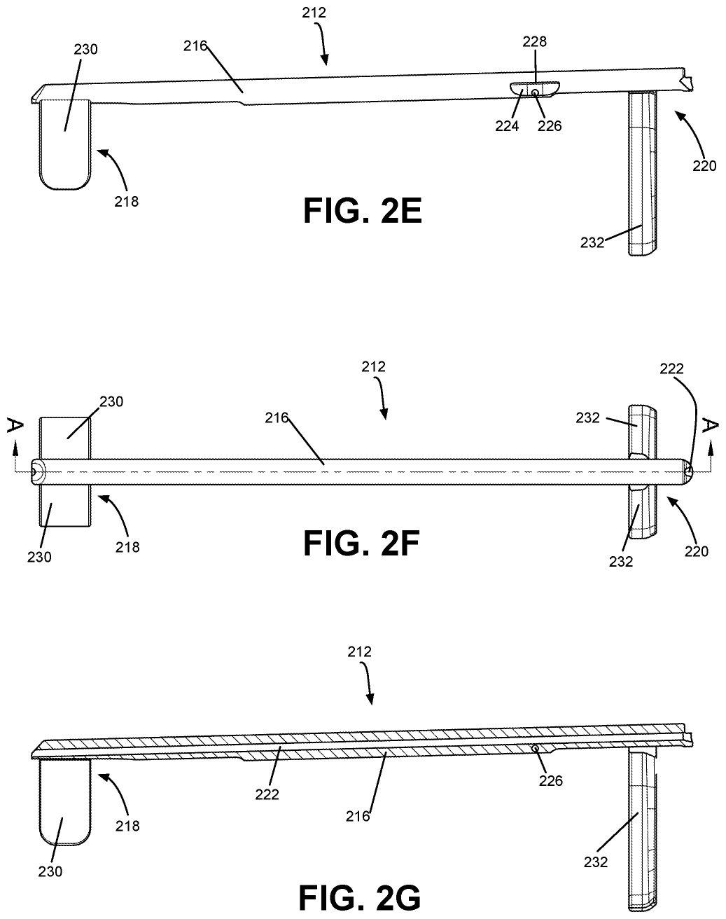

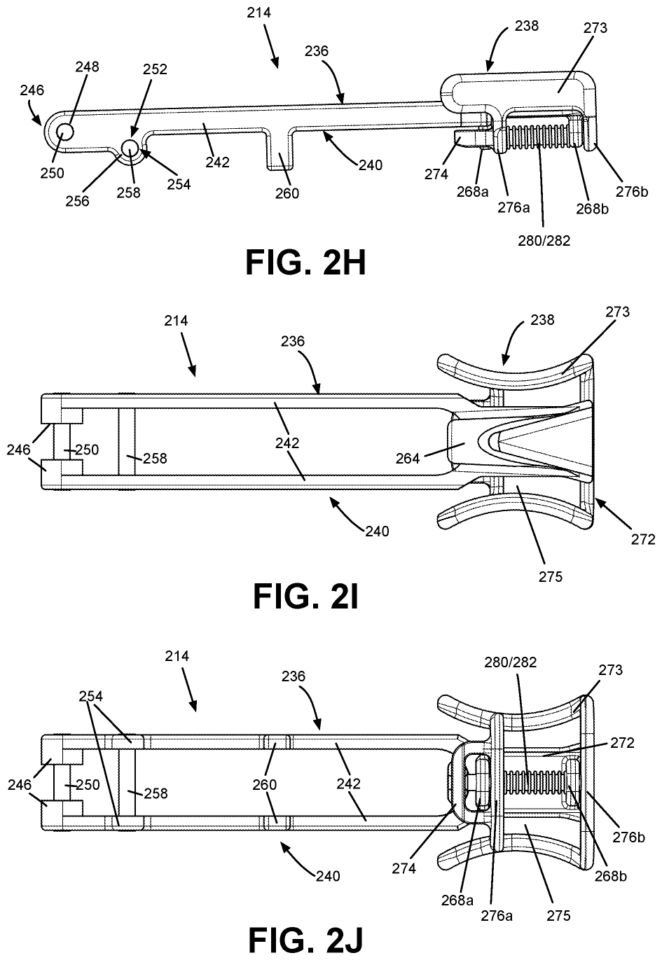

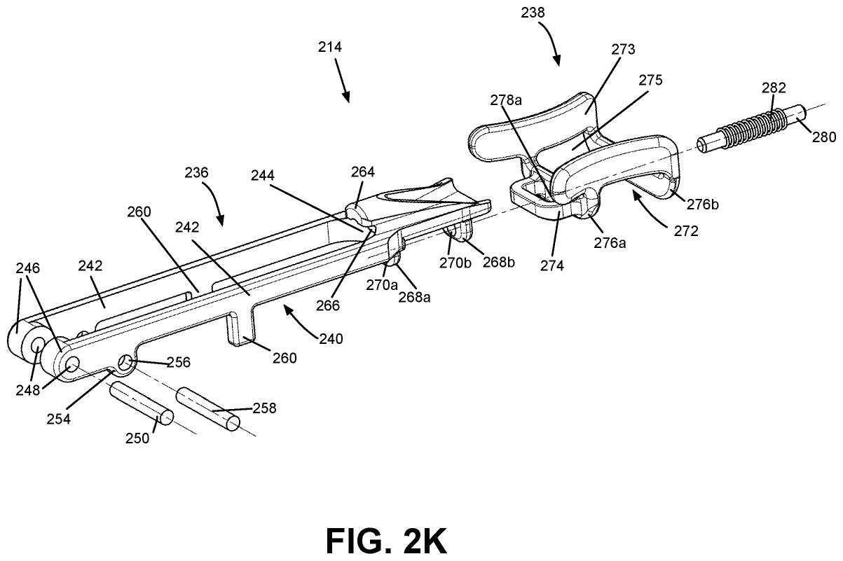

FIGS. 2A and 2B are top and left side views, respectively, illustrating one embodiment of a needle guidance device 210 for use with an ultrasound probe 200 in an assembled or locked configuration, consistent with embodiments described herein. FIG. 2C and 2D are left side plan and right rear isometric views, respectively, illustrating needle guidance device 210 and ultrasound probe 200 in an open or pre-assembled configuration.

Consistent with embodiments described herein, ultrasound probe 200 and needle guidance device 210 are configured to be lockingly coupled to each other so that, during use, needle guidance device 210 is fixed relative to ultrasound probe 200. As shown in FIGS. 2A-2D ultrasound probe 200 includes one or more features that facilitate cooperative engagement with needle guidance device 210. In particular, ultrasound probe 200 includes a generally tubular body 201 having a forward end 202, an intermediate portion 203, and a rearward end 204. As shown, forward end 202 includes a generally bulbous portion having a diameter larger than that of intermediate portion 203. Rearward end 204 includes a handle portion 205 and a transition portion 206 to transition a shape of body 201 between handle portion 205 and intermediate portion 203. As shown, transition portion 206 includes a least a portion having a larger diameter than that of intermediate portion 203 and handle portion 205.

Consistent with implementations described herein, transition portion 206 includes a pair of locating pockets 208 positioned on an upper surface of probe 200 forward of a portion of probe 200 having the largest diameter. Locating pockets 208 include a forward-facing cavity having a rear wall therein. As described in additional detail below, locating pockets 208 facilitate positioning and securing of needle guidance device 210 to ultrasound probe 200 during assembly.

Needle guidance device 210 includes a guide body 212 and a lock assembly 214. Guide body 212 includes a guide tube 216, a forward securing member 218, and a rear securing member 220. FIG. 2E-2G are left side, top, and cross-sectional views, respectively, of guide body 212. The cross-sectional view of FIG. 2G is taken along the line A-A in FIG. 2F.

As shown in FIGS. 2E-2G, guide tube 216 comprises a generally cylindrical configuration having a central aperture 222 extending therethrough and sized for receiving a needle or other puncture device. In some implementations, guide tubes 216 having different sized apertures 222 may be provided for different gauge puncture devices. In some embodiments, rather than an enclosed central aperture 222, guide tube 216 may include a channel or slot formed vertically therein, into which the puncture device is received.

An intermediate portion of guide tube 216 includes cut out portion 224 that includes a transverse aperture 226 extending therethrough and positioned below the central aperture 222 in guide tube 216, as shown in FIG. 2G. As described below, transverse aperture 226 is sized to receive a pivot pin when assembled together with lock assembly 214. In addition, cut out portion 224 includes a planar upper portion 228, which forms a stop for engaging a portion of lock assembly 214, as also described below.

Forward securing member 218 includes opposing curved arms 230 that project downwardly from a forward portion of guide tube 216 and which include a shape configured to correspond to a shape of forward end 202 of ultrasound probe 200. During assembly, curved arms 230 are configured to engage forward end 202 of ultrasound probe 200 to positively center guide tube 216 longitudinally with respect to ultrasound probe 200. Consistent with the embodiment of FIGS. 2A-G, curved arms 230 may be configured to extend more than 180°. In this manner, insertion of forward end 202 of ultrasound probe 200 within curved arms 230 effectively captures an axial position of the forward end of guide tube 216 relative to ultrasound probe 200. That is, the forward end of guide tube 216 may not be pulled radially outwardly from ultrasound probe 200 when forward end 202 of ultrasound probe 200 is positioned with curved arms 230.

Rear securing member 220 also includes curved arms 232 that project downwardly from guide tube 216 and which are configured to engage an outer surface of transition portion 206 of ultrasound probe 200. Consistent with the embodiment of FIGS. 2A-G, curved arms 232 may also be configured to extend more than 180°. In this manner, insertion of rearward end 202 of ultrasound probe 200 within curved arms 232 effectively captures an axial position of the rearward end of guide tube 216 relative to ultrasound probe 200. That is, the rearward end of guide tube 216 may not be pulled radially outwardly from ultrasound probe 200 when transition portion 206 of ultrasound probe 200 is positioned with curved arms 232. In addition, as shown in FIG. 2G, curved arms 232 of rear securing member 220 may be contoured to configure with a surface configuration of transition portion 206 so as to prevent forward longitudinal movement of guide body 212 relative to ultrasound probe 200 once curved arms 232 are engaged with transition portion 206.

FIGS. 2H-2K are left side, top, bottom, and exploded isometric views, respectively, of lock assembly 214 consistent with implementations described herein. As shown, lock assembly 214 includes a lever component 236 and a latch component 238 which together cooperatively engage guide tube 216 and ultrasound probe 200 to secure needle guidance device 200 to ultrasound probe 200 in the manner described below.

As shown in FIGS. 2I-2K, lever component 236 includes a body portion 240 having a pair of opposing arms 242 and a guide tube alignment portion 244. Arms 242 are sized to accommodate at least a portion of guide tube 216 therebetween, as shown in FIG. 2F. A forward end of each of arms 242 includes an engagement portion 246 for engaging cut out portion 224 in guide tube 216. As shown in FIGS. 2H and 2K, each engagement portion 246 includes an aperture 248 therethrough for aligning with transverse aperture 226 in cut out portion 224. During assembly, apertures 248 and 226 receive a pivot pin 250 therethrough.

Consistent with embodiments described herein, arms 242 also include a pivot limiting element 252 configured to limit the extent to which lever component 236 may be pivoted with respect to guide tube 216. In one implementation, pivot limiting element 252 includes downward projections 254 in arms 242 that include apertures 256 therethrough which may receive a limiting pin 258 therethrough. The position of apertures 256 relative to apertures 248 define the pivot limit for lock assembly 214. In other implementations, pivot limiting element 252 may include a different structure, such as a fixed bar which joins arms 242 in a location similar to limiting pin 258.

As shown in FIGS. 2H and 2K, arms 242 in lever component 236 may also include pocket engagement elements 260 which project downwardly therefrom. As shown in FIG. 2A, when locked onto ultrasound probe 200, pocket engagement elements 260 become seated within pockets 208. When pocket engagement elements 260 engage locating pockets 208 and rear securing member 220 positively engages transition portion 206 of ultrasound probe 200, needle guidance device becomes fixed relative to ultrasound probe 200.

As shown, a rearward end of lever component 236 includes a guide tube alignment portion 244 which joins arms 242. As shown in FIG. 2K, guide tube alignment portion 244 includes an upper guide tube engagement feature 264 and a central aperture 266 longitudinally positioned to align with central aperture 222 in guide tube 216, when latch component 238 is engaged with lever component 236 and guide tube 216, as described below. As shown in FIG. 2B, upper guide tube engagement feature 264 includes a portion which projects forwardly relative to a remainder of guide tube alignment portion 244. When lock assembly 214 is latched onto guide tube 216, upper guide tube engagement feature 264 is configured to engage an upper, rear end of guide tube 216 in a position in which aperture 266 is aligned with aperture 222.

As shown in FIGS. 2B, 2H, and 2K, guide tube alignment portion 244 further includes downwardly projecting tabs 268a and 268b. Tabs 268a and 268b include apertures 270a and 270b formed therethrough and function to engage a biasing element in latch component 238, as described below.

As shown in FIGS. 2I-2K and FIG. 2A-2B, latch component 238 comprises a generally frame-like member 272 configured to engage both a rearward portion guide tube 216 of and lever component 236. In particular, as shown, latch component 238 includes a latch engagement portion 273 and a lower guide tube engagement feature 274. Latch engagement portion 273 projects upwardly from frame-like member 272 and provides an engagement surface with which a user may manipulate (i.e., open and close) latch component 238, as described below. Frame-like member 272 include cavity 275 therein for receiving at least a portion of guide tube alignment portion 244, as shown in FIG. 2A. Frame-like member 272 includes a pair of tab engagement elements 276a and 276b that include alignment apertures 278a and 278b formed therethrough. Lower guide tube engagement feature 274 projects forwardly from forward tab engagement element 276a and engages a lower surface of guide tube 216 when latch component 238 is in a latched position with respect to guide tube 216.

During assembly, guide tube alignment portion 244 of lever component 236 is inserted with cavity 275 such that tab engagement elements 276a and 276b abut respective rearward surfaces of tabs 268a and 268b. A rod 280 having a biasing element, such as a spring, 282 positioned thereon is inserted and secured within apertures 270a/270b and 278a/278b, such that biasing element 282 is positioned between a rearward side of forward tab engagement element 276 and a forward side of rearward tab 268b, as shown in FIG. 2J.

To secure needle guidance device 210 to ultrasound probe 200, an operator positions guide tube 216 relative to ultrasound probe 200 such that forward securing member 218 and rearward securing member of guide tube 216 engage forward end 202 and transition portion 206 of ultrasound probe 200, respectively. Guide tube 216 is then fixed relative to ultrasound probe 200 by seating transition portion 206 within curved arms 232 of rear securing member 220.

Latch component 238 is then opened by pulling rearwardly on latch engagement portion 273, thus compressing biasing element 282 and allowing lower guide tube engagement feature 274 to move rearwardly relative to lever component 236. Lever component 236 is then pivoted about pivot pin 250 such that pocket engagement elements 268 become seated within locating pockets 208. Latch engagement portion 273 is then released, which causes biasing element 282 to urge lower guide tube engagement feature 274 to move forwardly relative to lever component 236. In its forward position, lower guide tube engagement feature 274 engages a lower surface of guide tube 216 and upper guide tube engagement feature 264 engages a lower surface of guide tube 216, effectively securing guide tube 216 to ultrasound probe 200.

Consistent with embodiments described herein, central aperture 266 in guide tube alignment portion 244 is only aligned with central aperture 222 in guide tube 216 when guide tube 216 is securely latched onto ultrasound probe, thus preventing premature or unsafe use of needle guidance device 210.

Upon completion of a procedure, needle guidance device 210 may be removed from ultrasound probe 200 by reversing the above steps. That is, latch engagement portion 273 may be again moved rearwardly to release guide tube 216 from lower guide tube engagement feature 274. Lever component 236 may then be pivoted about pivot pin 250 such that pocket engagement elements 268 becomes disengaged from locating pockets 208. At this point, needle guidance device 210 may be removed from ultrasound probe 200 by sliding guide body 212 forward relative to ultrasound probe 200.

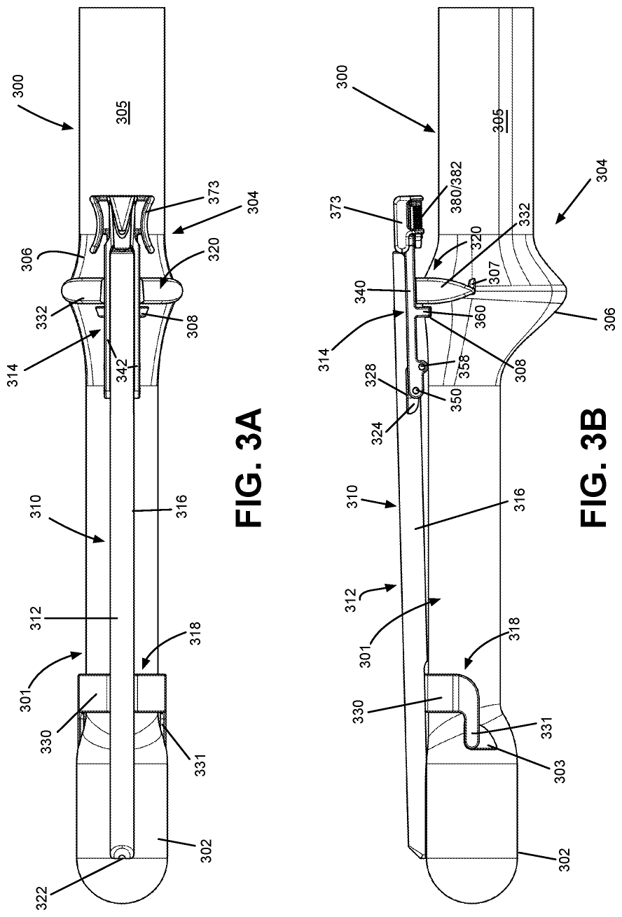

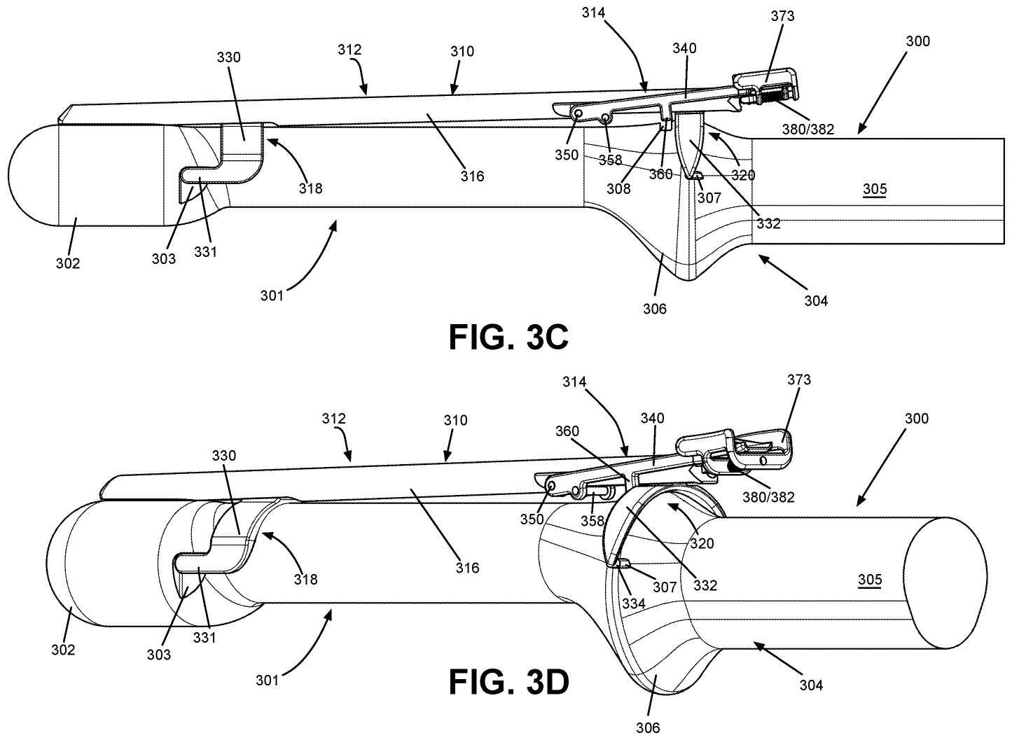

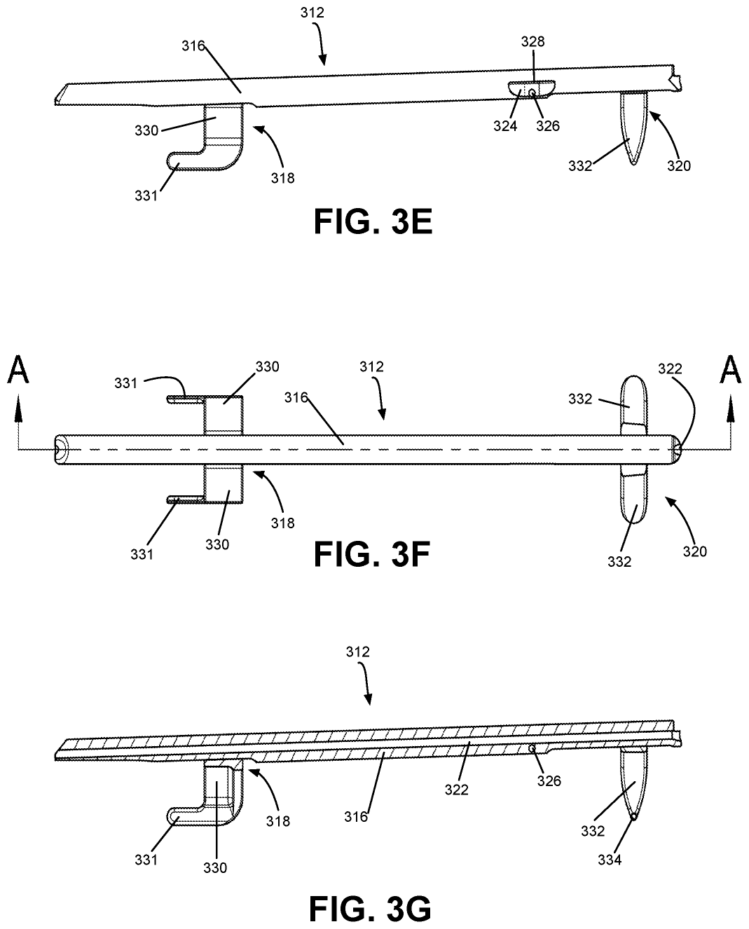

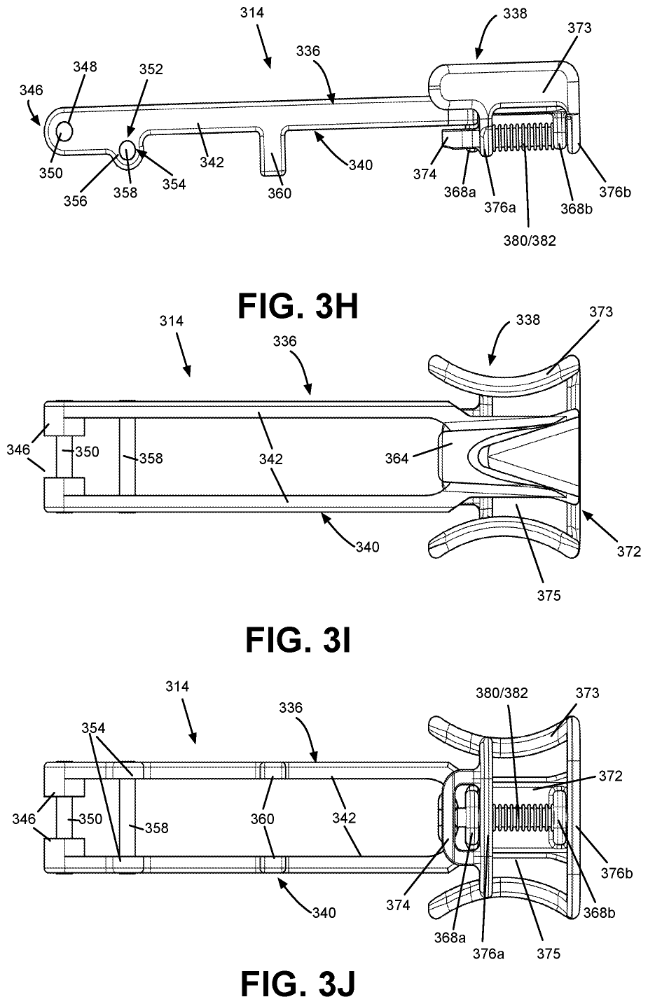

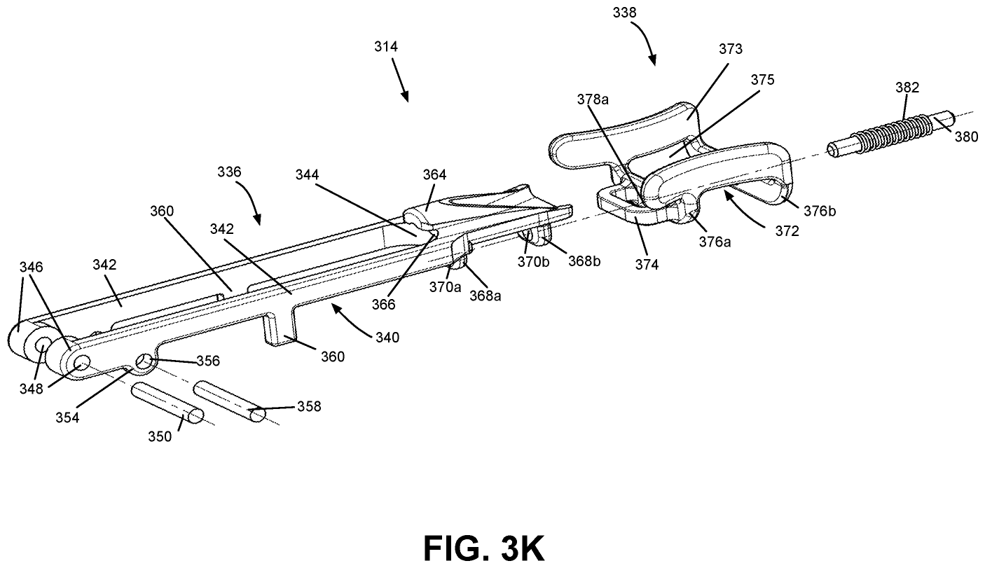

FIGS. 3A and 3B are top and left side views, respectively, illustrating one embodiment of a needle guidance device 310 for use with an ultrasound probe 300 in an assembled or locked configuration, consistent with embodiments described herein. FIG. 3C and 3D are left side plan and right rear isometric views, respectively, illustrating needle guidance device 310 and ultrasound probe 300 in an open or pre-assembled configuration.

Consistent with embodiments described herein, ultrasound probe 300 and needle guidance device 310 are configured to be lockingly coupled to each other so that, during use, needle guidance device 310 is fixed relative to ultrasound probe 300. As shown in FIGS. 3A-3D ultrasound probe 300 includes one or more features that facilitate cooperative engagement with needle guidance device 310. In particular, ultrasound probe 300 includes a generally tubular body 301 having a forward end 302 and a rearward end 304.

As shown, forward end 302 includes a generally bulbous portion having a diameter larger than that of the intermediate portion between the forward and rearward ends. Consistent with embodiments described herein, a rearward portion of forward end 302 includes a pair of forward locating pockets 303 positioned on opposite sides of probe 300. Forward locating pockets 303 include a rearward-facing cavity having a forward wall therein. As described in additional detail below, forward locating pockets 303 facilitate positioning and securing of needle guidance device 310 to ultrasound probe 300 during assembly.

Rearward end 304 includes a handle portion 305 and a transition portion 306 to transition a shape of body 301 between handle portion 305 and the intermediate portion. As shown, transition portion 306 includes a least a portion having a larger diameter than that of the intermediate portion and handle portion 305.

Consistent with implementations described herein, transition portion 306 includes a pair of rearward locating pockets 307 positioned on opposite sides of probe 300 and an upper locating slot 308 formed in a top surface of probe 300. Rearward locating pockets 307 include a rearward-facing cavity having a forward wall therein, which effectively act as stops for preventing further axial advancement of ultrasound probe 100 relative to needle guidance device 310. Upper locating slot 308 is configured to receive slot engagement members. As described in additional detail below, rearward locating pockets 307, in coordination with forward locating pockets 303 and upper locating slot 308, facilitate positioning and securing of needle guidance device 310 to ultrasound probe 300 during assembly.

Needle guidance device 310 includes a guide body 312 and a lock assembly 314. Guide body 312 includes a guide tube 316, a forward securing member 318, and a rear securing member 320. FIGS. 3E-3G are left side, top, and cross-sectional views, respectively, of guide body 312. The cross-sectional view of FIG. 3G is taken along the line A-A in FIG. 3F.

As shown in FIGS. 3E-3G, guide tube 316 comprises a generally cylindrical configuration having a central aperture 322 extending therethrough and sized for receiving a needle or other puncture device. In some implementations, guide tubes 316 having different sized apertures 322 may be provided for different gauge puncture devices. In some embodiments, rather than an enclosed central aperture 322, guide tube 316 may include a channel or slot formed vertically therein, into which the puncture device is received.

An intermediate portion of guide tube 316 includes cut out portion 324 that includes a transverse aperture 326 extending therethrough and positioned below the central aperture 322 in guide tube 316, as shown in FIG. 3G. As described below, transverse aperture 326 is sized to receive a pivot pin when assembled together with lock assembly 314. In addition, cut out portion 324 includes a planar upper portion 328, which forms a stop for engaging a portion of lock assembly 314, as also described below.

Forward securing member 318 includes opposing curved arms 330 that project downwardly from a forward portion of guide tube 316 and which include a shape configured to correspond to a shape of forward end 302 of ultrasound probe 300. During assembly, curved arms 330 are configured to engage forward end 302 of ultrasound probe 300 to positively center guide tube 316 longitudinally with respect to ultrasound probe 300. Consistent with the embodiment of FIGS. 3A-G, curved arms 330 terminate in forward pocket engaging members 331 for engaging forward locating pockets 303 in ultrasound probe 300. In one implementation, forward pocket engaging members 331 comprise a pair of arms that project forwardly and substantially perpendicularly from curved arms 330. As shown, forward pocket engaging members 331 are sized for receipt with forward locating pockets 303.

In this manner, insertion of forward end 302 of ultrasound probe 300 within curved arms 330 effectively captures an axial position of the forward end of guide tube 316 relative to ultrasound probe 300. That is, the forward end of guide tube 316 may not be pulled radially outwardly from ultrasound probe 300 when forward end 302 of ultrasound probe 300 is positioned with curved arms 330.

Rear securing member 320 also includes curved arms 332 that project downwardly from guide tube 316 and which are configured to engage an outer surface of transition portion 306 of ultrasound probe 300. Consistent with some implementations described herein, unlike curved arms 330 in forward securing member 318, curved arms 332 in rear securing member 320 include locating elements 334 which project inwardly at a terminus of curved arms 332. As shown in FIG. 3A, when assembled, locating elements 334 are slidingly positioned within locating pockets 307 in transition portion 306 of ultrasound probe 300.

FIGS. 3H-3K are left side, top, bottom, and exploded isometric views, respectively, of lock assembly 314 consistent with implementations described herein. As shown, lock assembly 314 includes a lever component 336 and a latch component 338 which together cooperatively engage guide tube 316 and ultrasound probe 300 to secure needle guidance device 300 to ultrasound probe 300 in the manner described below.

As shown in FIGS. 3I-3K, lever component 336 includes a body portion 340 having a pair of opposing arms 342 and a guide tube alignment portion 344. Arms 342 are sized to accommodate at least a portion of guide tube 316 therebetween, as shown in FIG. 3F. A forward end of each of arms 342 includes an engagement portion 346 for engaging cut out portion 324 in guide tube 316. As shown in FIGS. 3H and 3K, each engagement portion 346 includes an aperture 348 therethrough for aligning with transverse aperture 326 in cut out portion 324. During assembly, apertures 348 and 326 receive a pivot pin 350 therethrough.

Consistent with embodiments described herein, arms 342 also include a pivot limiting element 352 configured to limit the extent to which lever component 336 may be pivoted with respect to guide tube 316. In one implementation, pivot limiting element 352 includes downward projections 354 in arms 342 that include apertures 356 therethrough which may receive a limiting pin 358 therethrough. The position of apertures 356 relative to apertures 348 define the pivot limit for lock assembly 314. In other implementations, pivot limiting element 352 may include a different structure, such as a fixed bar which joins arms 342 in a location similar to limiting pin 358.

As shown in FIGS. 3H and 3K, arms 342 in lever component 336 may also include slot engagement elements 360 which project downwardly therefrom. As shown in FIG. 3A, when locked onto ultrasound probe 300, slot engagement elements 360 become seated within locating slot 308. When slot engagement elements 360 engage locating slot 308 and locating elements 334 in rear securing member 320 engage locating pockets 307, needle guidance device becomes fixed relative to ultrasound probe 300.

As shown, a rearward end of lever component 336 includes a guide tube alignment portion 344 which joins arms 342. As shown in FIG. 3K, guide tube alignment portion 344 includes an upper guide tube engagement feature 364 and a central aperture 366 longitudinally positioned to align with central aperture 322 in guide tube 316, when latch component 338 is engaged with lever component 336 and guide tube 316, as described below. As shown in FIG. 3B, upper guide tube engagement feature 364 includes a portion which projects forwardly relative to a remainder of guide tube alignment portion 344. When lock assembly 314 is latched onto guide tube 316, upper guide tube engagement feature 364 is configured to engage an upper, rear end of guide tube 316 in a position in which aperture 366 is aligned with aperture 322.

As shown in FIGS. 3B, 3H, and 3K, guide tube alignment portion 344 further includes downwardly projecting tabs 368a and 368b. Tabs 368a and 368b include apertures 370a and 370b formed therethrough and function to engage a biasing element in latch component 338, as described below.

As shown in FIGS. 3I-3K and FIG. 3A-3B, latch component 338 comprises a generally frame-like member 372 configured to engage both a rearward portion guide tube 316 of and lever component 336. In particular, as shown, latch component 338 includes a latch engagement portion 373 and a lower guide tube engagement feature 374. Latch engagement portion 373 projects upwardly from frame-like member 372 and provides an engagement surface with which a user may manipulate (i.e., open and close) latch component 338, as described below. Frame-like member 372 include cavity 375 therein for receiving at least a portion of guide tube alignment portion 344, as shown in FIG. 3A. Frame-like member 372 includes a pair of tab engagement elements 376a and 376b that include alignment apertures 378a and 378b formed therethrough. Lower guide tube engagement feature 374 projects forwardly from forward tab engagement element 376a and engages a lower surface of guide tube 316 when latch component 338 is in a latched position with respect to guide tube 316.

During assembly, guide tube alignment portion 344 of lever component 336 is inserted with cavity 375 such that tab engagement elements 376a and 376b abut respective rearward surfaces of tabs 368a and 368b. A rod 380 having a biasing element, such as a spring, 382 positioned thereon is inserted and secured within apertures 370a/370b and 378a/378b, such that biasing element 382 is positioned between a rearward side of forward tab engagement element 376 and a forward side of rearward tab 368b, as shown in FIG. 3J.

To secure needle guidance device 310 to ultrasound probe 300, an operator positions guide tube 316 relative to ultrasound probe 300 such that forward securing member 318 and rearward securing member of guide tube 316 engage forward end 302 and transition portion 306 of ultrasound probe 300, respectively. Guide tube 316 is then fixed relative to ultrasound probe 300 by seating transition portion 306 within curved arms 332 of rear securing member 320.

Latch component 338 is then opened by pulling rearwardly on latch engagement portion 373, thus compressing biasing element 382 and allowing lower guide tube engagement feature 374 to move rearwardly relative to lever component 336. Lever component 336 is then pivoted about pivot pin 350 such that slot engagement elements 368 become seated within locating slot 308. Latch engagement portion 373 is then released, which causes biasing element 382 to urge lower guide tube engagement feature 374 to move forwardly relative to lever component 336. In its forward position, lower guide tube engagement feature 374 engages a lower surface of guide tube 316 and upper guide tube engagement feature 364 engages a lower surface of guide tube 316, effectively securing guide tube 316 to ultrasound probe 300.

Consistent with embodiments described herein, central aperture 366 in guide tube alignment portion 344 is only aligned with central aperture 322 in guide tube 316 when guide tube 316 is securely latched onto ultrasound probe, thus preventing premature or unsafe use of needle guidance device 310.

Upon completion of a procedure, needle guidance device 310 may be removed from ultrasound probe 300 by reversing the above steps. That is, latch engagement portion 373 may be again moved rearwardly to release guide tube 316 from lower guide tube engagement feature 374. Lever component 336 may then be pivoted about pivot pin 350 such that slot engagement elements 368 becomes disengaged from locating slot 308. At this point, needle guidance device 310 may be removed from ultrasound probe 300 by sliding guide body 312 forward relative to ultrasound probe 300.

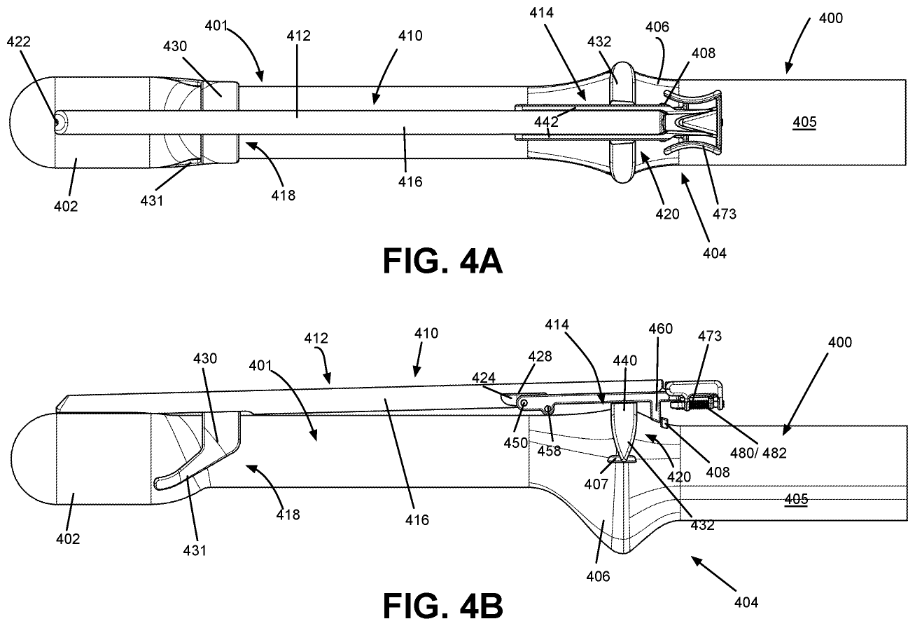

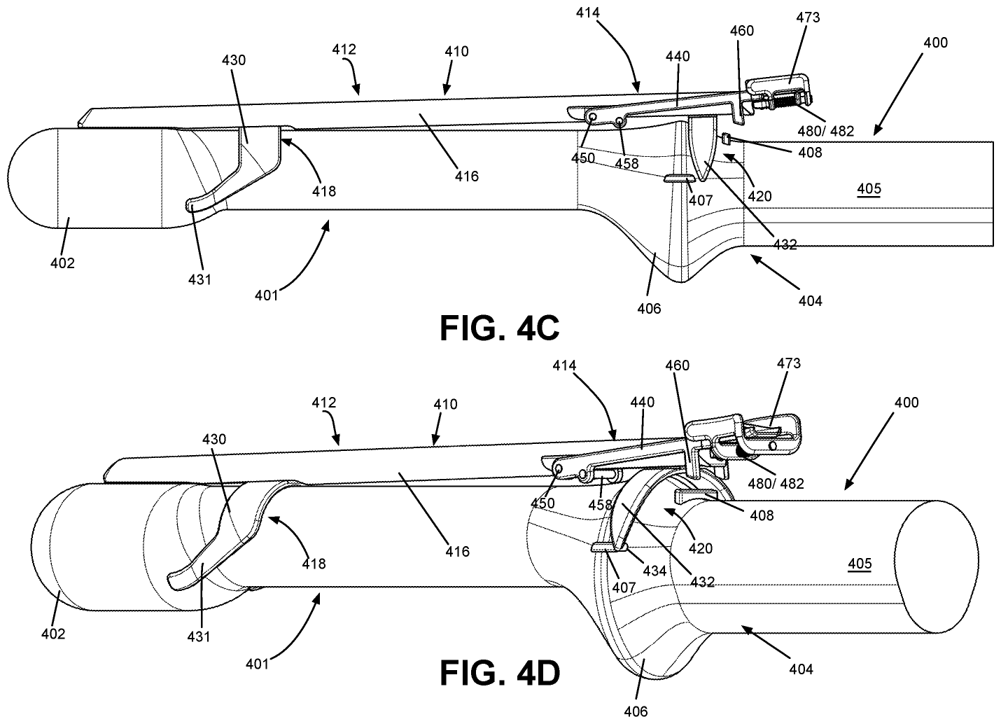

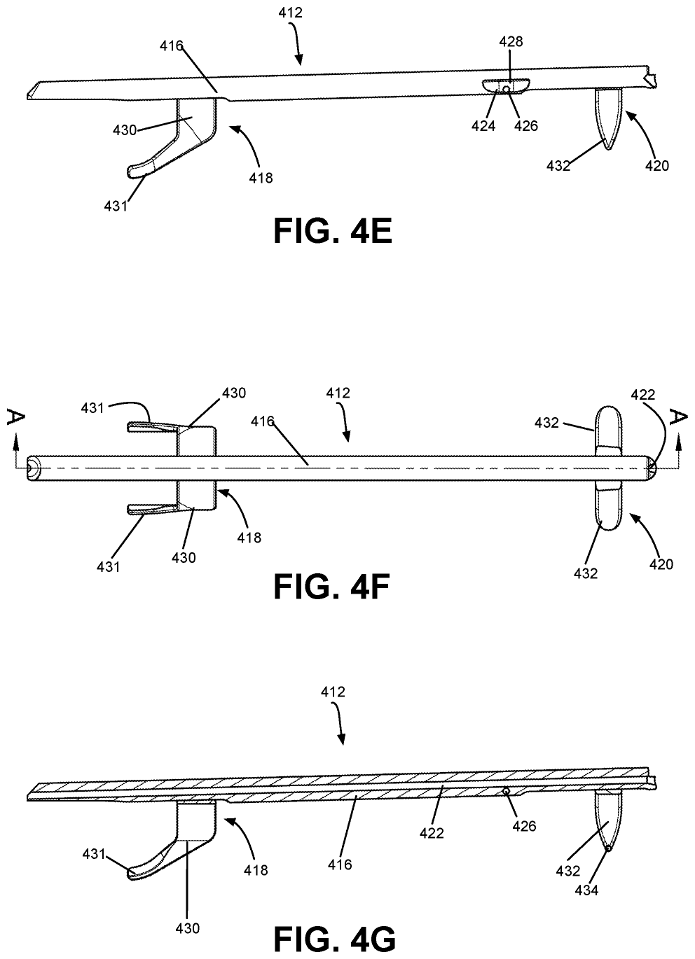

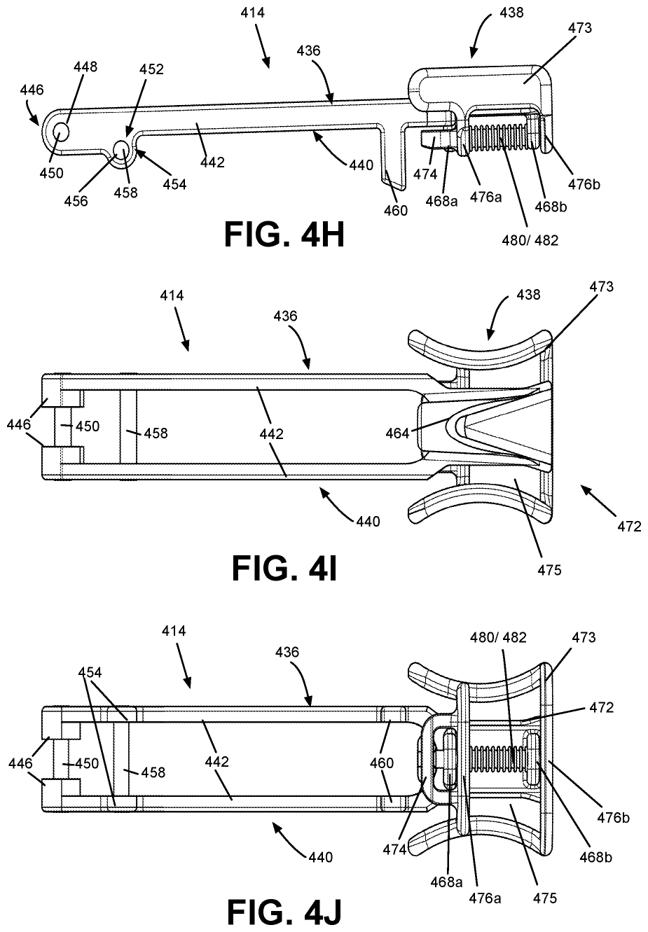

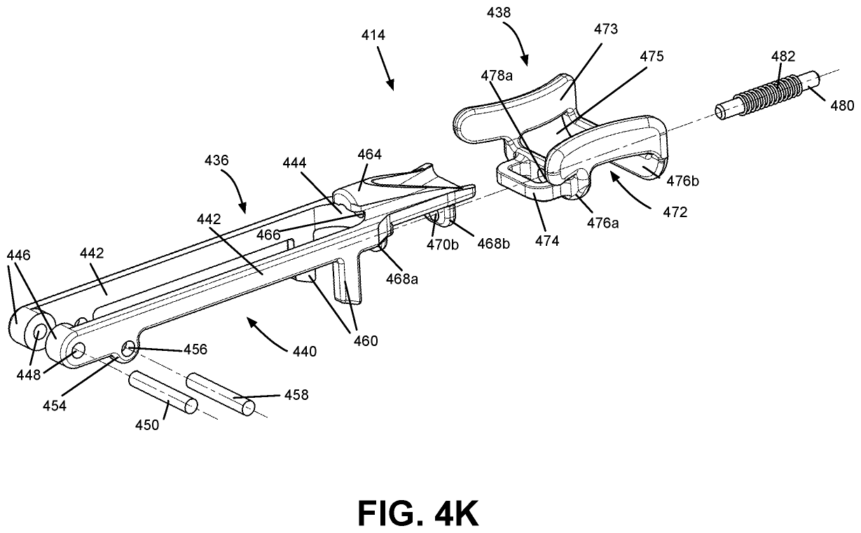

FIGS. 4A and 4B are top and left side views, respectively, illustrating one embodiment of a needle guidance device 410 for use with an ultrasound probe 400 in an assembled or locked configuration, consistent with embodiments described herein. FIG. 4C and 4D are left side plan and right rear isometric views, respectively, illustrating needle guidance device 410 and ultrasound probe 400 in an open or pre-assembled configuration.

Consistent with embodiments described herein, ultrasound probe 400 and needle guidance device 410 are configured to be lockingly coupled to each other so that, during use, needle guidance device 410 is fixed relative to ultrasound probe 400. As shown in FIGS. 4A-4D ultrasound probe 400 includes one or more features that facilitate cooperative engagement with needle guidance device 410. In particular, ultrasound probe 400 includes a generally tubular body 401 having a forward end 402, an intermediate portion, and a rearward end 404.

As shown, forward end 402 includes a generally bulbous portion having a diameter larger than that of the intermediate portion between the forward and rearward ends. Rearward end 404 includes a handle portion 405 and a transition portion 406 to transition a shape of body 401 between handle portion 405 and intermediate portion 403. As shown, transition portion 406 includes a least a portion having a larger diameter than that of intermediate portion 403 and handle portion 405.

Consistent with implementations described herein, transition portion 406 includes a pair of locating pockets 407 positioned on opposite sides of probe 400 and a locating rib 408 formed in a top surface of probe 400. Locating pockets 407 include a rearward-facing cavity having a forward wall therein. Locating rib 408 is configured to engage a portion of needle guidance device 410 during use, as described below. However, as shown in FIG. 4A, contrary to the embodiment of FIGS. 4A-4K, locating rib 408 is positioned on a portion of transition portion 406 rearward of locating pockets 407. As described below, locating pockets 407, in coordination with locating rib 408, facilitate positioning and securing of needle guidance device 410 to ultrasound probe 400 during assembly.

Needle guidance device 410 includes a guide body 412 and a lock assembly 414. Guide body 412 includes a guide tube 416, a forward securing member 418, and a rear securing member 420. FIGS. 4E-4G are left side, top, and cross-sectional views, respectively, of guide body 412. The cross-sectional view of FIG. 4G is taken along the line A-A in FIG. 4F.

As shown in FIGS. 4E-4G, guide tube 416 comprises a generally cylindrical configuration having a central aperture 422 extending therethrough and sized for receiving a needle or other puncture device. In some implementations, guide tubes 416 having different sized apertures 422 may be provided for different gauge puncture devices. In some embodiments, rather than an enclosed central aperture 422, guide tube 416 may include a channel or slot formed vertically therein, into which the puncture device is received.

An intermediate portion of guide tube 416 includes cut out portion 424 that includes a transverse aperture 426 extending therethrough and positioned below the central aperture 422 in guide tube 416, as shown in FIG. 4G. As described below, transverse aperture 426 is sized to receive a pivot pin when assembled together with lock assembly 414. In addition, cut out portion 424 includes a planar upper portion 428, which forms a stop for engaging a portion of lock assembly 414, as also described below.

Forward securing member 418 includes opposing curved arms 430 that project downwardly from a forward portion of guide tube 416 and which include a shape configured to correspond to a shape of forward end 402 of ultrasound probe 400. As shown in FIG. 4B, curved arms 430 may include forward facing prongs 431 contoured to correspond to a shape of the rearward side of forward end 402 to positively position needle guidance device 410 longitudinally relative to ultrasound probe during use, as described below.

In this manner, insertion of forward end 402 of ultrasound probe 400 within curved arms 430 effectively captures an axial position of the forward end of guide tube 416 relative to ultrasound probe 400. That is, the forward end of guide tube 416 may not be pulled radially outwardly from ultrasound probe 400 when forward end 402 of ultrasound probe 400 is positioned with curved arms 430.

Rear securing member 420 also includes curved arms 432 that project downwardly from guide tube 416 and which are configured to engage an outer surface of transition portion 406 of ultrasound probe 400. Consistent with some implementations described herein, unlike curved arms 430 in forward securing member 418, curved arms 432 in rear securing member 420 include locating elements 434 which project inwardly at a terminus of curved arms 432. As shown in FIG. 4A, when assembled, locating elements 434 are slidingly positioned within locating pockets 407 in transition portion 406 of ultrasound probe 400.

FIGS. 4H-4K are left side, top, bottom, and exploded isometric views, respectively, of lock assembly 414 consistent with implementations described herein. As shown, lock assembly 414 includes a lever component 436 and a latch component 438 which together cooperatively engage guide tube 416 and ultrasound probe 400 to secure needle guidance device 400 to ultrasound probe 400 in the manner described below.

As shown in FIGS. 4I-4K, lever component 436 includes a body portion 440 having a pair of opposing arms 442 and a guide tube alignment portion 444. Arms 442 are sized to accommodate at least a portion of guide tube 416 therebetween, as shown in FIG. 4F. A forward end of each of arms 442 includes an engagement portion 446 for engaging cut out portion 424 in guide tube 416. As shown in FIGS. 4H and 4K, each engagement portion 446 includes an aperture 448 therethrough for aligning with transverse aperture 426 in cut out portion 424. During assembly, apertures 448 and 426 receive a pivot pin 450 therethrough.

Consistent with embodiments described herein, arms 442 also include a pivot limiting element 452 configured to limit the extent to which lever component 436 may be pivoted with respect to guide tube 416. In one implementation, pivot limiting element 452 includes downward projections 454 in arms 442 that include apertures 456 therethrough which may receive a limiting pin 458 therethrough. The position of apertures 456 relative to apertures 448 define the pivot limit for lock assembly 414. In other implementations, pivot limiting element 452 may include a different structure, such as a fixed bar which joins arms 442 in a location similar to limiting pin 458.

As shown in FIGS. 4H and 4K, arms 442 in lever component 436 may also include rib engagement elements 460 which project downwardly therefrom. As shown in FIG. 4A, when locked onto ultrasound probe 400, rib engagement elements 460 engage a forward side of locating rib 408. When rib engagement elements 460 engage locating rib 408 and locating elements 434 in rear securing member 420 engage locating pockets 407, needle guidance device becomes fixed relative to ultrasound probe 400.

As shown, a rearward end of lever component 436 includes a guide tube alignment portion 444 which joins arms 442. As shown in FIG. 4K, guide tube alignment portion 444 includes an upper guide tube engagement feature 464 and a central aperture 466 longitudinally positioned to align with central aperture 422 in guide tube 416, when latch component 438 is engaged with lever component 436 and guide tube 416, as described below. As shown in FIG. 4B, upper guide tube engagement feature 464 includes a portion which projects forwardly relative to a remainder of guide tube alignment portion 444. When lock assembly 414 is latched onto guide tube 416, upper guide tube engagement feature 464 is configured to engage an upper, rear end of guide tube 416 in a position in which aperture 466 is aligned with aperture 422.

As shown in FIGS. 4B, 4H, and 4K, guide tube alignment portion 444 further includes downwardly projecting tabs 468a and 468b. Tabs 468a and 468b include apertures 470a and 470b formed therethrough and function to engage a biasing element in latch component 438, as described below.

As shown in FIGS. 41-4K and FIG. 4A-4B, latch component 438 comprises a generally frame-like member 472 configured to engage both a rearward portion guide tube 416 of and lever component 436. In particular, as shown, latch component 438 includes a latch engagement portion 473 and a lower guide tube engagement feature 474. Latch engagement portion 473 projects upwardly from frame-like member 472 and provides an engagement surface with which a user may manipulate (i.e., open and close) latch component 438, as described below. Frame-like member 472 include cavity 475 therein for receiving at least a portion of guide tube alignment portion 444, as shown in FIG. 4A. Frame-like member 472 includes a pair of tab engagement elements 476a and 476b that include alignment apertures 478a and 478b formed therethrough. Lower guide tube engagement feature 474 projects forwardly from forward tab engagement element 476a and engages a lower surface of guide tube 416 when latch component 438 is in a latched position with respect to guide tube 416.

During assembly, guide tube alignment portion 444 of lever component 436 is inserted with cavity 475 such that tab engagement elements 476a and 476b abut respective rearward surfaces of tabs 468a and 468b. A rod 480 having a biasing element, such as a spring, 482 positioned thereon is inserted and secured within apertures 470a/470b and 478a/478b, such that biasing element 482 is positioned between a rearward side of forward tab engagement element 476 and a forward side of rearward tab 468b, as shown in FIG. 4J.

To secure needle guidance device 410 to ultrasound probe 400, an operator positions guide tube 416 relative to ultrasound probe 400 such that forward securing member 418 and rearward securing member of guide tube 416 engage forward end 402 and transition portion 406 of ultrasound probe 400, respectively. Guide tube 416 is then fixed relative to ultrasound probe 400 by seating transition portion 406 within curved arms 432 of rear securing member 420.

Latch component 438 is then opened by pulling rearwardly on latch engagement portion 473, thus compressing biasing element 482 and allowing lower guide tube engagement feature 474 to move rearwardly relative to lever component 436. Lever component 436 is then pivoted about pivot pin 450 such that slot engagement elements 468 become seated within locating slot 408. Latch engagement portion 473 is then released, which causes biasing element 482 to urge lower guide tube engagement feature 474 to move forwardly relative to lever component 436. In its forward position, lower guide tube engagement feature 474 engages a lower surface of guide tube 416 and upper guide tube engagement feature 464 engages a lower surface of guide tube 416, effectively securing guide tube 416 to ultrasound probe 400.

Consistent with embodiments described herein, central aperture 466 in guide tube alignment portion 444 is only aligned with central aperture 422 in guide tube 416 when guide tube 416 is securely latched onto ultrasound probe, thus preventing premature or unsafe use of needle guidance device 410.

Upon completion of a procedure, needle guidance device 410 may be removed from ultrasound probe 400 by reversing the above steps. That is, latch engagement portion 473 may be again moved rearwardly to release guide tube 416 from lower guide tube engagement feature 474. Lever component 436 may then be pivoted about pivot pin 450 such that slot engagement elements 468 becomes disengaged from locating slot 408. At this point, needle guidance device 410 may be removed from ultrasound probe 400 by sliding guide body 412 forward relative to ultrasound probe 400.

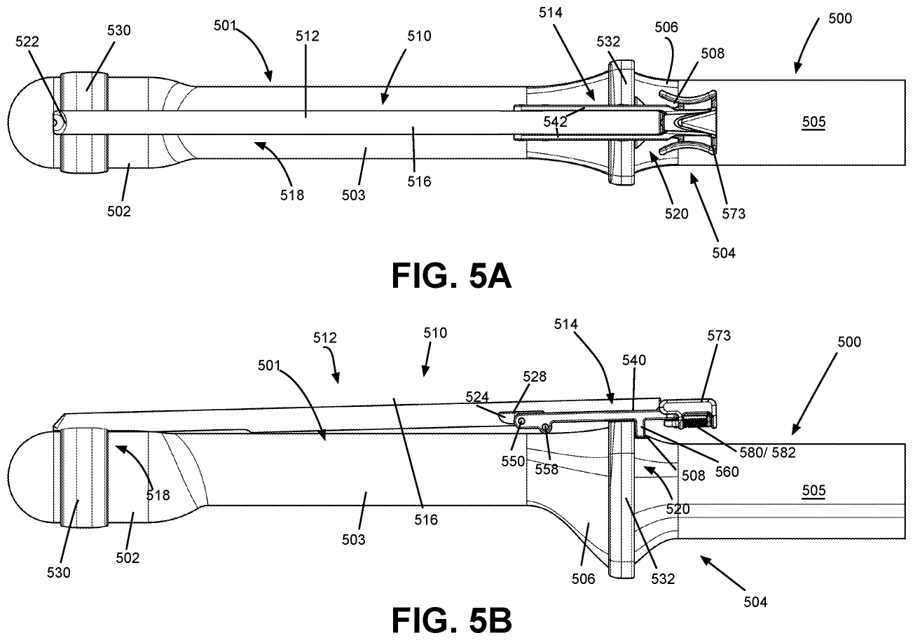

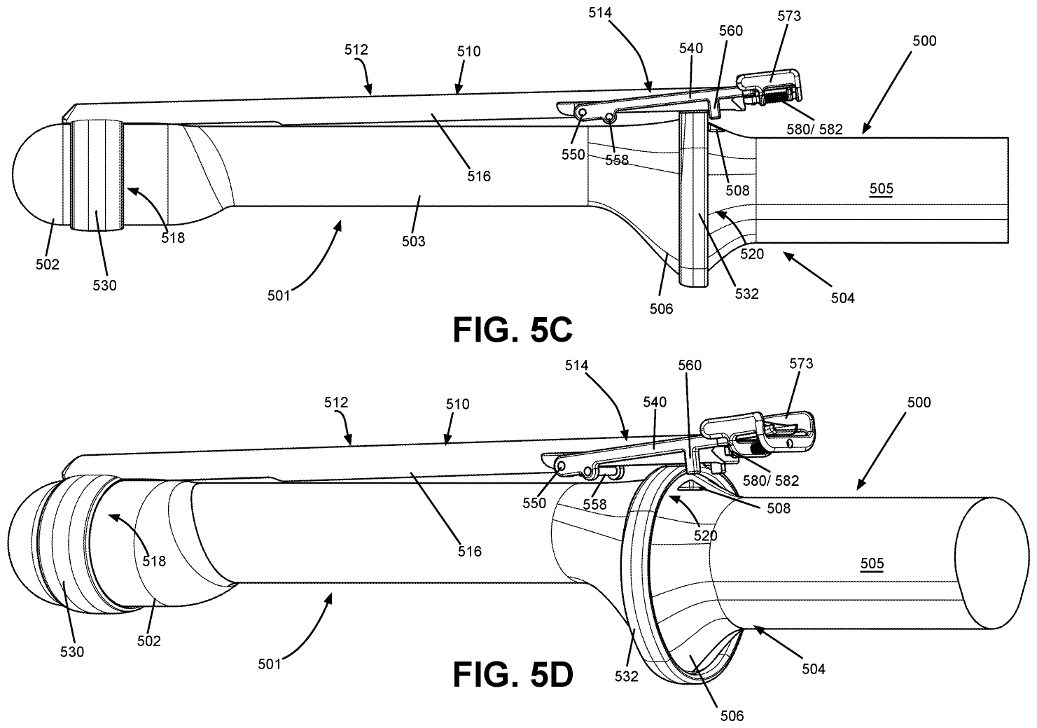

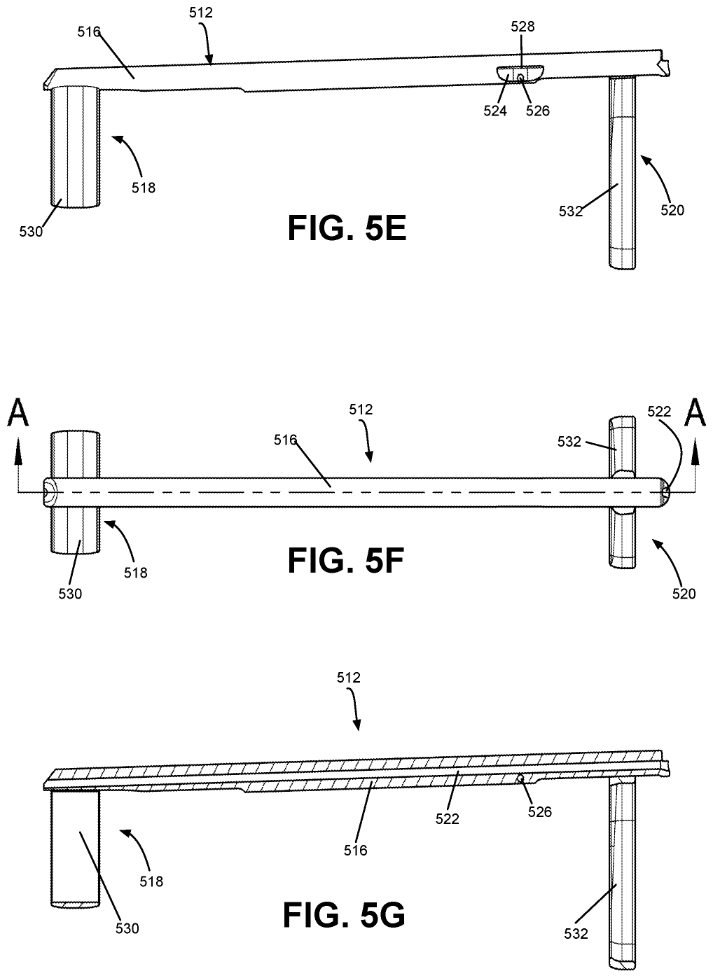

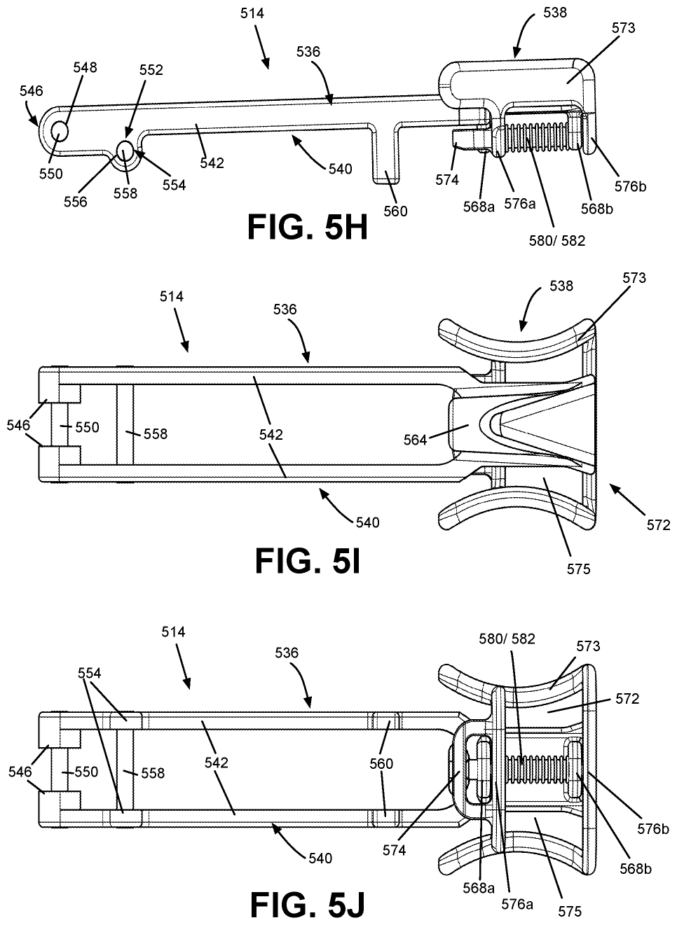

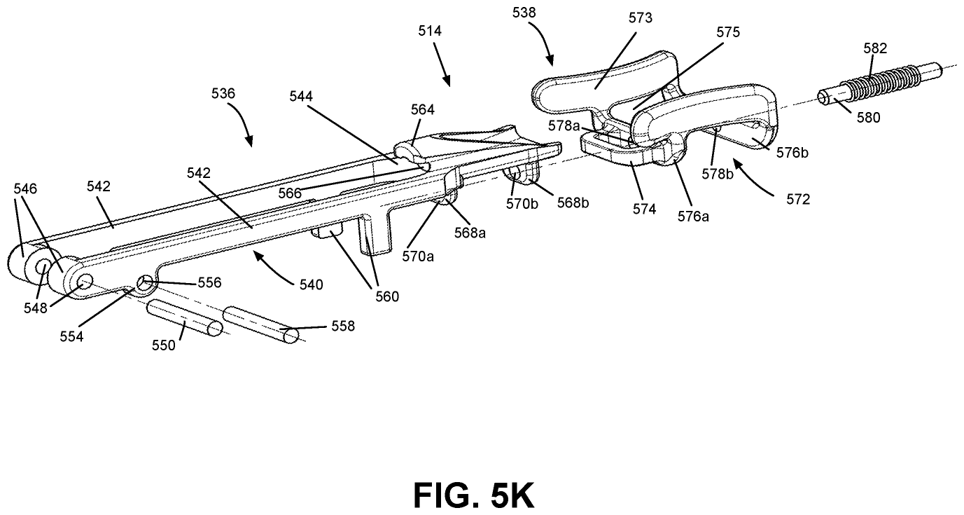

FIGS. 5A and 5B are top and left side views, respectively, illustrating one embodiment of a needle guidance device 510 for use with an ultrasound probe 500 in an assembled or locked configuration, consistent with embodiments described herein. FIG. 5C and 5D are left side plan and right rear isometric views, respectively, illustrating needle guidance device 510 and ultrasound probe 500 in an open or pre-assembled configuration.

Consistent with embodiments described herein, ultrasound probe 500 and needle guidance device 510 are configured to be lockingly coupled to each other so that, during use, needle guidance device 510 is fixed relative to ultrasound probe 500. As shown in FIGS. 5A-5D ultrasound probe 500 includes one or more features that facilitate cooperative engagement with needle guidance device 510. In particular, ultrasound probe 500 includes a generally tubular body 501 having a forward end 502, an intermediate portion, and a rearward end 504.

As shown, forward end 502 includes a generally bulbous portion having a diameter larger than that of the intermediate portion between the forward and rearward ends. Rearward end 504 includes a handle portion 505 and a transition portion 506 to transition a shape of body 501 between handle portion 505 and intermediate portion 503. As shown, transition portion 506 includes a least a portion having a larger diameter than that of intermediate portion 503 and handle portion 505.

Consistent with implementations described herein, transition portion 506 includes a pair of locating pockets 508 positioned on a top surface of probe 500 rearward of a portion of probe 500 having the largest diameter. Locating pockets 508 include a rearward-facing cavity having a forward wall therein. As described below, locating pockets 508 facilitate positioning and securing of needle guidance device 510 to ultrasound probe 500 during assembly.

Needle guidance device 510 includes a guide body 512 and a lock assembly 514. Guide body 512 includes a guide tube 516, a forward securing member 518, and a rear securing member 520. FIGS. 5E-5G are left side, top, and cross-sectional views, respectively, of guide body 512. The cross-sectional view of FIG. 5G is taken along the line A-A in FIG. 5F.

As shown in FIGS. 5E-5G, guide tube 516 comprises a generally cylindrical configuration having a central aperture 522 extending therethrough and sized for receiving a needle or other puncture device. In some implementations, guide tubes 516 having different sized apertures 522 may be provided for different gauge puncture devices. In some embodiments, rather than an enclosed central aperture 522, guide tube 516 may include a channel or slot formed vertically therein, into which the puncture device is received.

An intermediate portion of guide tube 516 includes cut out portion 524 that includes a transverse aperture 526 extending therethrough and positioned below the central aperture 522 in guide tube 516, as shown in FIG. 5G. As described below, transverse aperture 526 is sized to receive a pivot pin when assembled together with lock assembly 514. In addition, cut out portion 524 includes a planar upper portion 528, which forms a stop for engaging a portion of lock assembly 514, as also described below.

Consistent with implementations described herein, forward securing member 518 includes a ring configuration 530 that projects downwardly from a forward portion of guide tube 516 and which includes a shape configured to correspond to a shape of forward end 502 of ultrasound probe 500 to capture forward end 502 of ultrasound probe 500 within forward securing member 518 during use.

In this embodiment, rear securing member 520 also includes a ring configuration 532 that projects downwardly from guide tube 516 and which is configured to engage a portion of transition portion 506 of ultrasound probe 500 having its maximum diameter. In addition, as shown in FIG. 5G, rear securing member 520 may be contoured to configure with a surface configuration of transition portion 506 so as to prevent forward longitudinal movement of guide body 512 relative to ultrasound probe 500 once rear securing member 520 is engaged with transition portion 506.

FIGS. 5H-5K are left side, top, bottom, and exploded isometric views, respectively, of lock assembly 514 consistent with implementations described herein. As shown, lock assembly 514 includes a lever component 536 and a latch component 538 which together cooperatively engage guide tube 516 and ultrasound probe 500 to secure needle guidance device 500 to ultrasound probe 500 in the manner described below.

As shown in FIGS. 5I-5K, lever component 536 includes a body portion 540 having a pair of opposing arms 542 and a guide tube alignment portion 544. Arms 542 are sized to accommodate at least a portion of guide tube 516 therebetween, as shown in FIG. 5F. A forward end of each of arms 542 includes an engagement portion 546 for engaging cut out portion 524 in guide tube 516. As shown in FIGS. 5H and 5K, each engagement portion 546 includes an aperture 548 therethrough for aligning with transverse aperture 526 in cut out portion 524. During assembly, apertures 548 and 526 receive a pivot pin 550 therethrough.

Consistent with embodiments described herein, arms 542 also include a pivot limiting element 552 configured to limit the extent to which lever component 536 may be pivoted with respect to guide tube 516. In one implementation, pivot limiting element 552 includes downward projections 554 in arms 542 that include apertures 556 therethrough which may receive a limiting pin 558 therethrough. The position of apertures 556 relative to apertures 548 define the pivot limit for lock assembly 514. In other implementations, pivot limiting element 552 may include a different structure, such as a fixed bar which joins arms 542 in a location similar to limiting pin 558.

As shown in FIGS. 5H and 5K, arms 542 in lever component 536 may also include pocket engagement elements 560 which project downwardly therefrom. As shown in FIG. 5A, when locked onto ultrasound probe 500, pocket engagement elements 560 become seated within locating pockets 508. When pocket engagement elements 560 engage locating pockets 508 and rear securing member 520 positively engages transition portion 506 of ultrasound probe 500, needle guidance device becomes fixed relative to ultrasound probe 200.

As shown, a rearward end of lever component 536 includes a guide tube alignment portion 544 which joins arms 542. As shown in FIG. 5K, guide tube alignment portion 544 includes an upper guide tube engagement feature 564 and a central aperture 566 longitudinally positioned to align with central aperture 522 in guide tube 516, when latch component 538 is engaged with lever component 536 and guide tube 516, as described below. As shown in FIG. 5B, upper guide tube engagement feature 564 includes a portion which projects forwardly relative to a remainder of guide tube alignment portion 544. When lock assembly 514 is latched onto guide tube 516, upper guide tube engagement feature 564 is configured to engage an upper, rear end of guide tube 516 in a position in which aperture 566 is aligned with aperture 522.

As shown in FIGS. 5B, 5H, and 5K, guide tube alignment portion 544 further includes downwardly projecting tabs 568a and 568b. Tabs 568a and 568b include apertures 570a and 570b formed therethrough and function to engage a biasing element in latch component 538, as described below.

As shown in FIGS. 51-5K and FIG. 5A-5B, latch component 538 comprises a generally frame-like member 572 configured to engage both a rearward portion guide tube 516 of and lever component 536. In particular, as shown, latch component 538 includes a latch engagement portion 573 and a lower guide tube engagement feature 574. Latch engagement portion 573 projects upwardly from frame-like member 572 and provides an engagement surface with which a user may manipulate (i.e., open and close) latch component 538, as described below. Frame-like member 572 include cavity 575 therein for receiving at least a portion of guide tube alignment portion 544, as shown in FIG. 5A. Frame-like member 572 includes a pair of tab engagement elements 576a and 576b that include alignment apertures 578a and 578b formed therethrough. Lower guide tube engagement feature 574 projects forwardly from forward tab engagement element 576a and engages a lower surface of guide tube 516 when latch component 538 is in a latched position with respect to guide tube 516.

During assembly, guide tube alignment portion 544 of lever component 536 is inserted with cavity 575 such that tab engagement elements 576a and 576b abut respective rearward surfaces of tabs 568a and 568b. A rod 580 having a biasing element, such as a spring, 582 positioned thereon is inserted and secured within apertures 570a/570b and 578a/578b, such that biasing element 582 is positioned between a rearward side of forward tab engagement element 576 and a forward side of rearward tab 568b, as shown in FIG. 5J.

To secure needle guidance device 510 to ultrasound probe 500, an operator positions guide tube 516 relative to ultrasound probe 500 such that forward securing member 518 and rearward securing member of guide tube 516 engage forward end 502 and transition portion 506 of ultrasound probe 500, respectively. Guide tube 516 is then fixed relative to ultrasound probe 500 by seating transition portion 506 within curved ring 532 of rear securing member 520.

Latch component 538 is then opened by pulling rearwardly on latch engagement portion 573, thus compressing biasing element 582 and allowing lower guide tube engagement feature 574 to move rearwardly relative to lever component 536. Lever component 536 is then pivoted about pivot pin 550 such that pocket engagement elements 560 become seated within locating pockets 508. Latch engagement portion 573 is then released, which causes biasing element 582 to urge lower guide tube engagement feature 574 to move forwardly relative to lever component 536. In its forward position, lower guide tube engagement feature 574 engages a lower surface of guide tube 516 and upper guide tube engagement feature 564 engages a lower surface of guide tube 516, effectively securing guide tube 516 to ultrasound probe 500.

Consistent with embodiments described herein, central aperture 566 in guide tube alignment portion 544 is only aligned with central aperture 522 in guide tube 516 when guide tube 516 is securely latched onto ultrasound probe, thus preventing premature or unsafe use of needle guidance device 510.

Upon completion of a procedure, needle guidance device 510 may be removed from ultrasound probe 500 by reversing the above steps. That is, latch engagement portion 573 may be again moved rearwardly to release guide tube 516 from lower guide tube engagement feature 574. Lever component 536 may then be pivoted about pivot pin 550 such that pocket engagement elements 560 becomes disengaged from locating pockets 508. At this point, needle guidance device 510 may be removed from ultrasound probe 500 by sliding guide body 512 forward relative to ultrasound probe 500.

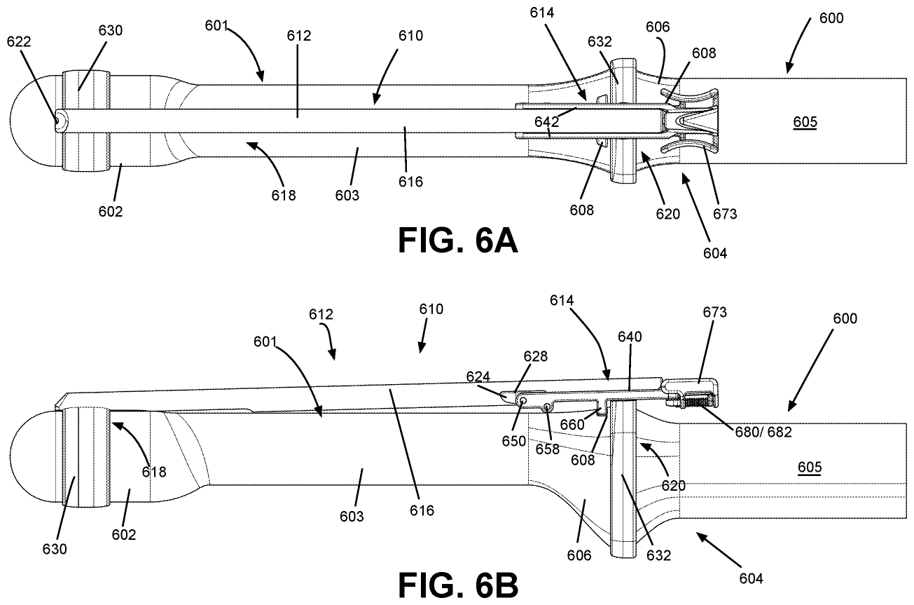

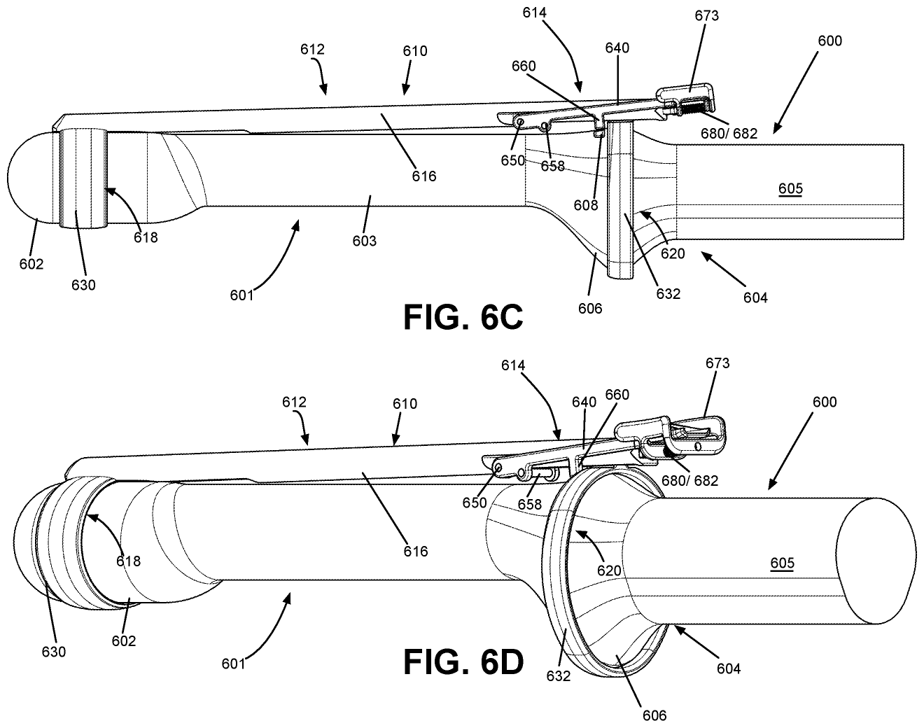

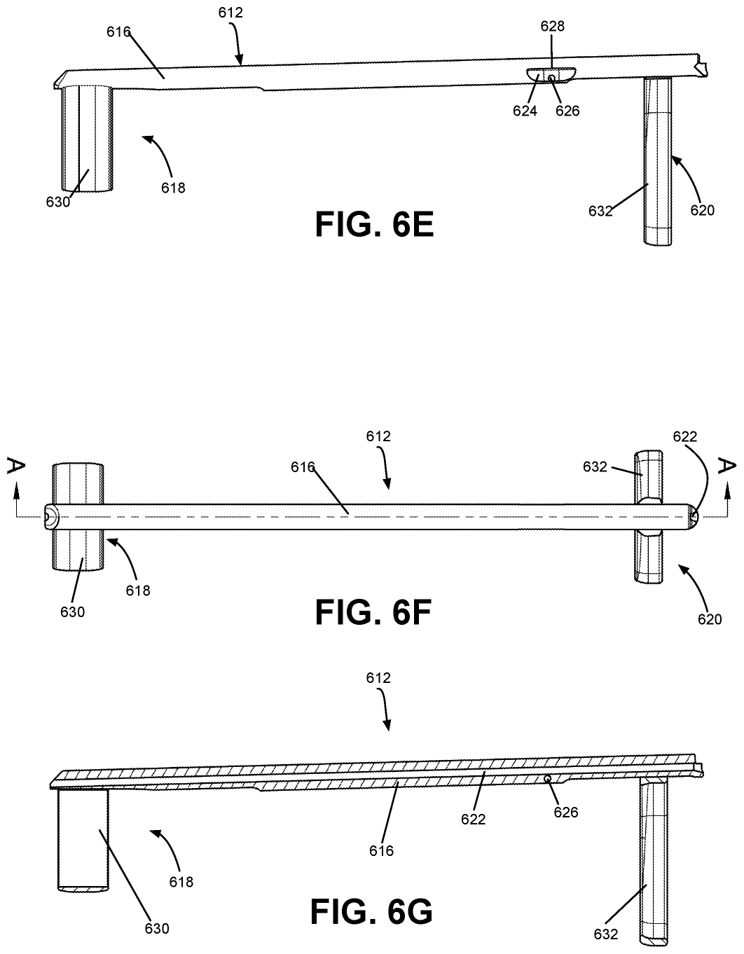

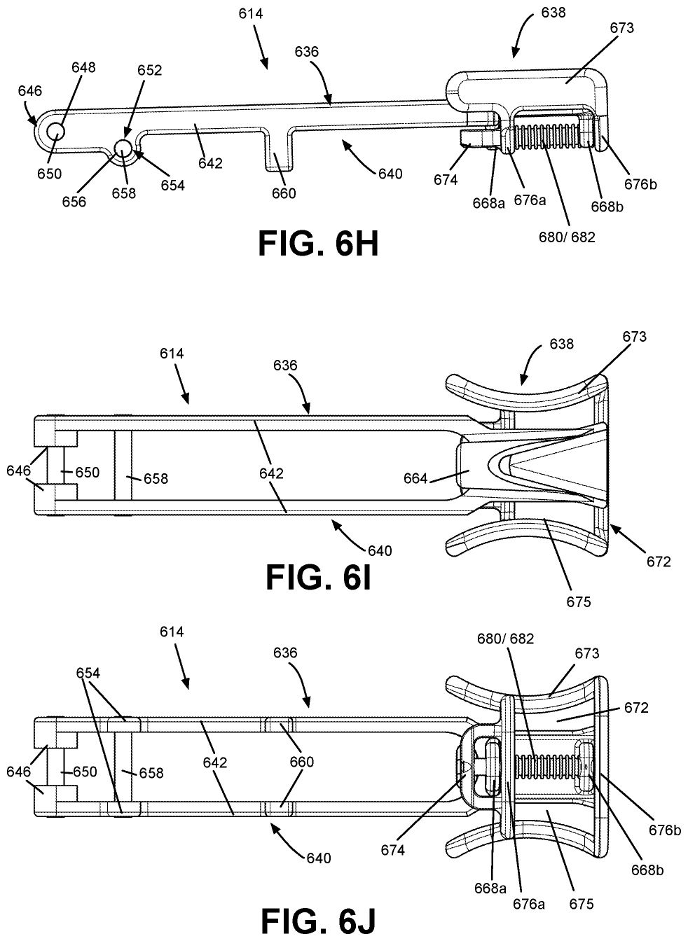

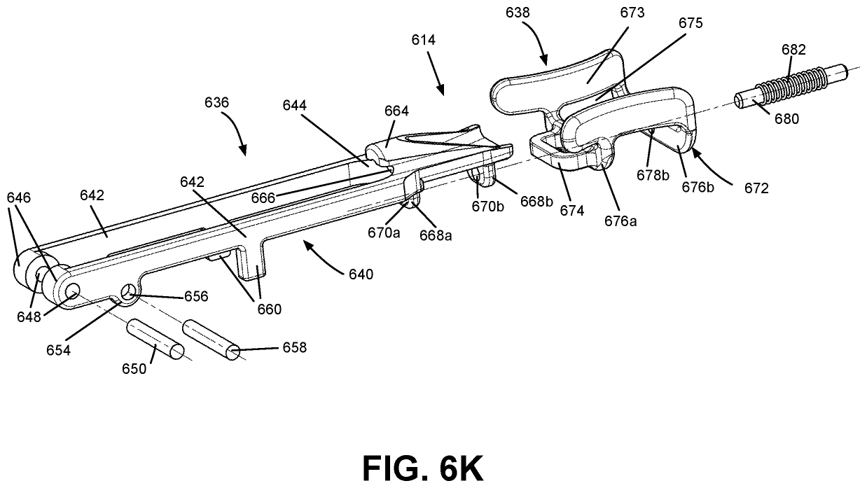

FIGS. 6A and 6B are top and left side views, respectively, illustrating one embodiment of a needle guidance device 610 for use with an ultrasound probe 600 in an assembled or locked configuration, consistent with embodiments described herein. FIG. 6C and 6D are left side plan and right rear isometric views, respectively, illustrating needle guidance device 610 and ultrasound probe 600 in an open or pre-assembled configuration.

Consistent with embodiments described herein, ultrasound probe 600 and needle guidance device 610 are configured to be lockingly coupled to each other so that, during use, needle guidance device 610 is fixed relative to ultrasound probe 600. As shown in FIGS. 6A-6D ultrasound probe 600 includes one or more features that facilitate cooperative engagement with needle guidance device 610. In particular, ultrasound probe 600 includes a generally tubular body 601 having a forward end 602, an intermediate portion, and a rearward end 604.

As shown, forward end 602 includes a generally bulbous portion having a diameter larger than that of the intermediate portion between the forward and rearward ends. Rearward end 604 includes a handle portion 605 and a transition portion 606 to transition a shape of body 601 between handle portion 605 and intermediate portion 603. As shown, transition portion 606 includes a least a portion having a larger diameter than that of intermediate portion 603 and handle portion 605.

Consistent with implementations described herein, transition portion 606 includes an upper locating slot 608 formed in a top surface of probe 300 forward of a portion of probe 600 having the largest diameter. Upper locating slot 308 is configured to receive slot engagement members in the lock assembly, as described below.

Needle guidance device 610 includes a guide body 612 and a lock assembly 614. Guide body 612 includes a guide tube 616, a forward securing member 618, and a rear securing member 620. FIGS. 6E-6G are left side, top, and cross-sectional views, respectively, of guide body 612. The cross-sectional view of FIG. 6G is taken along the line A-A in FIG. 6F.

As shown in FIGS. 6E-6G, guide tube 616 comprises a generally cylindrical configuration having a central aperture 622 extending therethrough and sized for receiving a needle or other puncture device. In some implementations, guide tubes 616 having different sized apertures 622 may be provided for different gauge puncture devices. In some embodiments, rather than an enclosed central aperture 622, guide tube 616 may include a channel or slot formed vertically therein, into which the puncture device is received.

An intermediate portion of guide tube 616 includes cut out portion 624 that includes a transverse aperture 626 extending therethrough and positioned below the central aperture 622 in guide tube 616, as shown in FIG. 6G. As described below, transverse aperture 626 is sized to receive a pivot pin when assembled together with lock assembly 614. In addition, cut out portion 624 includes a planar upper portion 628, which forms a stop for engaging a portion of lock assembly 614, as also described below.

Consistent with implementations described herein, forward securing member 618 includes a ring configuration 630 that projects downwardly from a forward portion of guide tube 616 and which includes a shape configured to correspond to a shape of forward end 602 of ultrasound probe 600 to capture forward end 602 of ultrasound probe 600 within forward securing member 618 during use.

In this embodiment, rear securing member 620 also includes a ring configuration 632 that projects downwardly from guide tube 616 and which is configured to engage a portion of transition portion 606 of ultrasound probe 600 having its maximum diameter. In addition, as shown in FIG. 6G, rear securing member 620 may be contoured to configure with a surface configuration of transition portion 606 so as to prevent forward longitudinal movement of guide body 612 relative to ultrasound probe 600 once rear securing member 620 is engaged with transition portion 606.

FIGS. 6H-6K are left side, top, bottom, and exploded isometric views, respectively, of lock assembly 614 consistent with implementations described herein. As shown, lock assembly 614 includes a lever component 636 and a latch component 638 which together cooperatively engage guide tube 616 and ultrasound probe 600 to secure needle guidance device 600 to ultrasound probe 600 in the manner described below.

As shown in FIGS. 6I-6K, lever component 636 includes a body portion 640 having a pair of opposing arms 642 and a guide tube alignment portion 644. Arms 642 are sized to accommodate at least a portion of guide tube 616 therebetween, as shown in FIG. 6F. A forward end of each of arms 642 includes an engagement portion 646 for engaging cut out portion 444 in guide tube 616. As shown in FIGS. 6H and 6K, each engagement portion 646 includes an aperture 648 therethrough for aligning with transverse aperture 446 in cut out portion 444. During assembly, apertures 648 and 446 receive a pivot pin 650 therethrough.

Consistent with embodiments described herein, arms 642 also include a pivot limiting element 652 configured to limit the extent to which lever component 636 may be pivoted with respect to guide tube 616. In one implementation, pivot limiting element 652 includes downward projections 654 in arms 642 that include apertures 656 therethrough which may receive a limiting pin 658 therethrough. The position of apertures 656 relative to apertures 648 define the pivot limit for lock assembly 614. In other implementations, pivot limiting element 652 may include a different structure, such as a fixed bar which joins arms 642 in a location similar to limiting pin 658.

As shown in FIGS. 6H and 6K, arms 642 in lever component 636 may also include slot engagement elements 660 which project downwardly therefrom. As shown in FIG. 6A, when locked onto ultrasound probe 600, slot engagement elements 660 become seated within locating slot 608. When pocket engagement elements 660 engage locating slot 608 and rear securing member 620 positively engages transition portion 606 of ultrasound probe 600, needle guidance device becomes fixed relative to ultrasound probe 200.

As shown, a rearward end of lever component 636 includes a guide tube alignment portion 644 which joins arms 642. As shown in FIG. 6K, guide tube alignment portion 644 includes an upper guide tube engagement feature 664 and a central aperture 666 longitudinally positioned to align with central aperture 442 in guide tube 616, when latch component 638 is engaged with lever component 636 and guide tube 616, as described below. As shown in FIG. 6B, upper guide tube engagement feature 664 includes a portion which projects forwardly relative to a remainder of guide tube alignment portion 644. When lock assembly 614 is latched onto guide tube 616, upper guide tube engagement feature 664 is configured to engage an upper, rear end of guide tube 616 in a position in which aperture 666 is aligned with aperture 442.

As shown in FIGS. 6B, 6H, and 6K, guide tube alignment portion 644 further includes downwardly projecting tabs 668a and 668b. Tabs 668a and 668b include apertures 670a and 670b formed therethrough and function to engage a biasing element in latch component 638, as described below.

As shown in FIGS. 6I-6K and FIG. 6A-6B, latch component 638 comprises a generally frame-like member 672 configured to engage both a rearward portion guide tube 616 of and lever component 636. In particular, as shown, latch component 638 includes a latch engagement portion 673 and a lower guide tube engagement feature 674. Latch engagement portion 673 projects upwardly from frame-like member 672 and provides an engagement surface with which a user may manipulate (i.e., open and close) latch component 638, as described below. Frame-like member 672 include cavity 675 therein for receiving at least a portion of guide tube alignment portion 644, as shown in FIG. 6A. Frame-like member 672 includes a pair of tab engagement elements 676a and 676b that include alignment apertures 678a and 678b formed therethrough. Lower guide tube engagement feature 674 projects forwardly from forward tab engagement element 676a and engages a lower surface of guide tube 616 when latch component 638 is in a latched position with respect to guide tube 616.

During assembly, guide tube alignment portion 644 of lever component 636 is inserted with cavity 675 such that tab engagement elements 676a and 676b abut respective rearward surfaces of tabs 668a and 668b. A rod 680 having a biasing element, such as a spring, 682 positioned thereon is inserted and secured within apertures 670a/670b and 678a/678b, such that biasing element 682 is positioned between a rearward side of forward tab engagement element 676 and a forward side of rearward tab 668b, as shown in FIG. 6J.

To secure needle guidance device 610 to ultrasound probe 600, an operator positions guide tube 616 relative to ultrasound probe 600 such that forward securing member 618 and rearward securing member of guide tube 616 engage forward end 602 and transition portion 606 of ultrasound probe 600, respectively. Guide tube 616 is then fixed relative to ultrasound probe 600 by seating transition portion 606 within curved arms 632 of rear securing member 620.

Latch component 638 is then opened by pulling rearwardly on latch engagement portion 673, thus compressing biasing element 682 and allowing lower guide tube engagement feature 674 to move rearwardly relative to lever component 636. Lever component 636 is then pivoted about pivot pin 650 such that slot engagement elements 660 become seated within locating slot 608. Latch engagement portion 673 is then released, which causes biasing element 682 to urge lower guide tube engagement feature 674 to move forwardly relative to lever component 636. In its forward position, lower guide tube engagement feature 674 engages a lower surface of guide tube 616 and upper guide tube engagement feature 664 engages a lower surface of guide tube 616, effectively securing guide tube 616 to ultrasound probe 600.

Consistent with embodiments described herein, central aperture 666 in guide tube alignment portion 644 is only aligned with central aperture 442 in guide tube 616 when guide tube 616 is securely latched onto ultrasound probe, thus preventing premature or unsafe use of needle guidance device 610.

Upon completion of a procedure, needle guidance device 610 may be removed from ultrasound probe 600 by reversing the above steps. That is, latch engagement portion 673 may be again moved rearwardly to release guide tube 616 from lower guide tube engagement feature 674. Lever component 636 may then be pivoted about pivot pin 650 such that slot engagement elements 660 becomes disengaged from locating slot 608. At this point, needle guidance device 610 may be removed from ultrasound probe 600 by sliding guide body 612 forward relative to ultrasound probe 600.

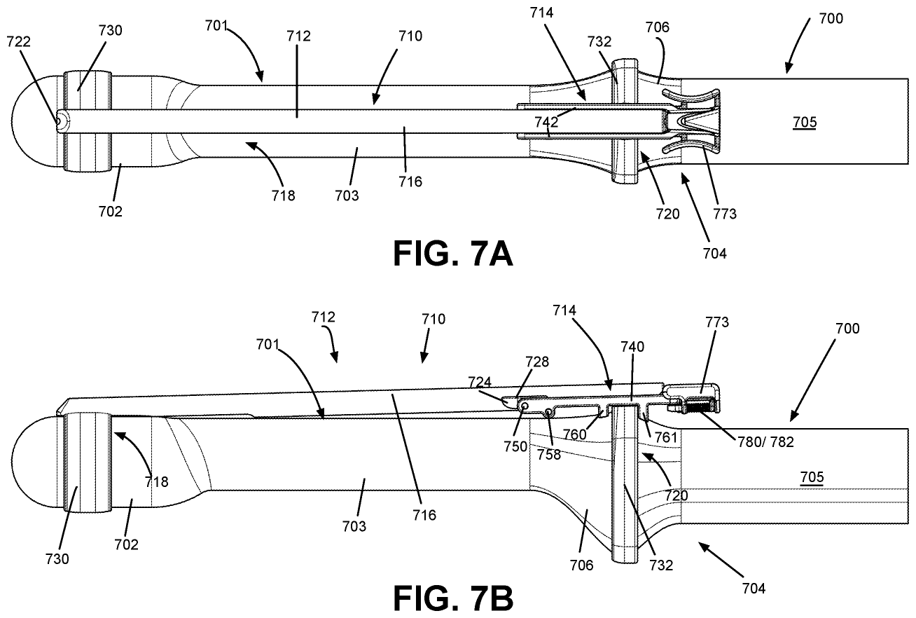

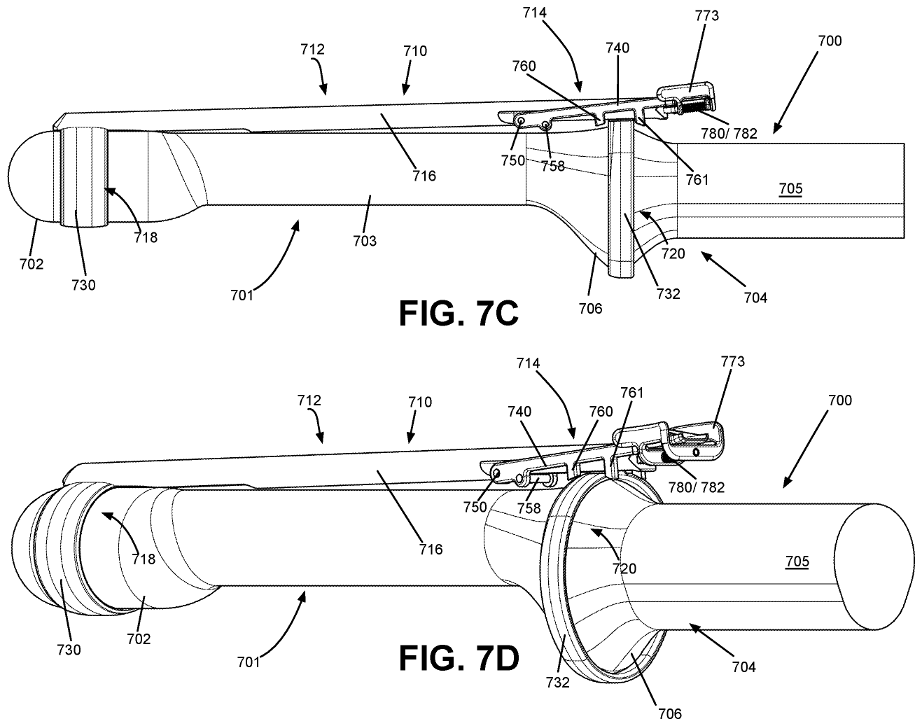

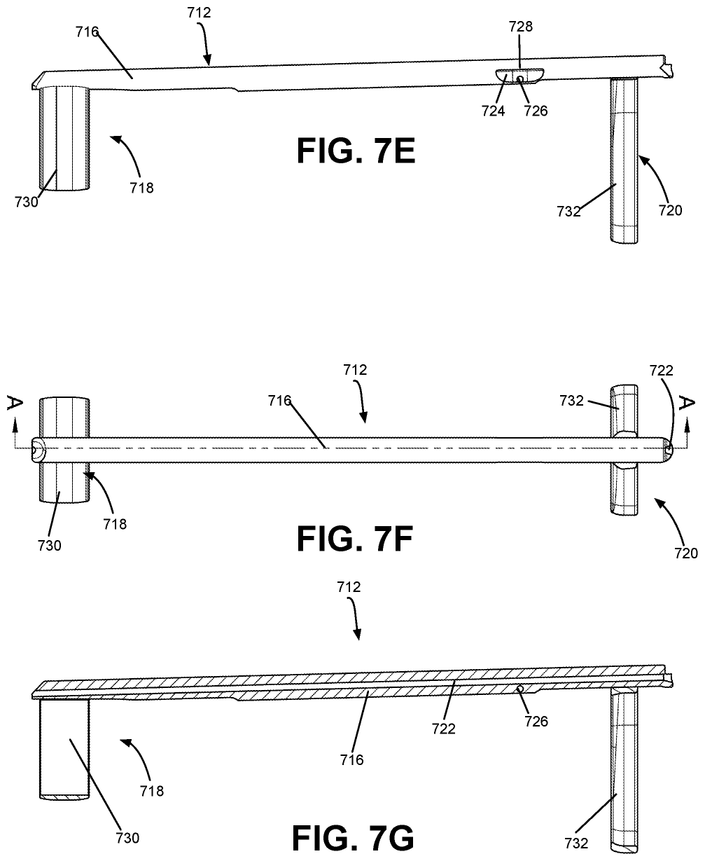

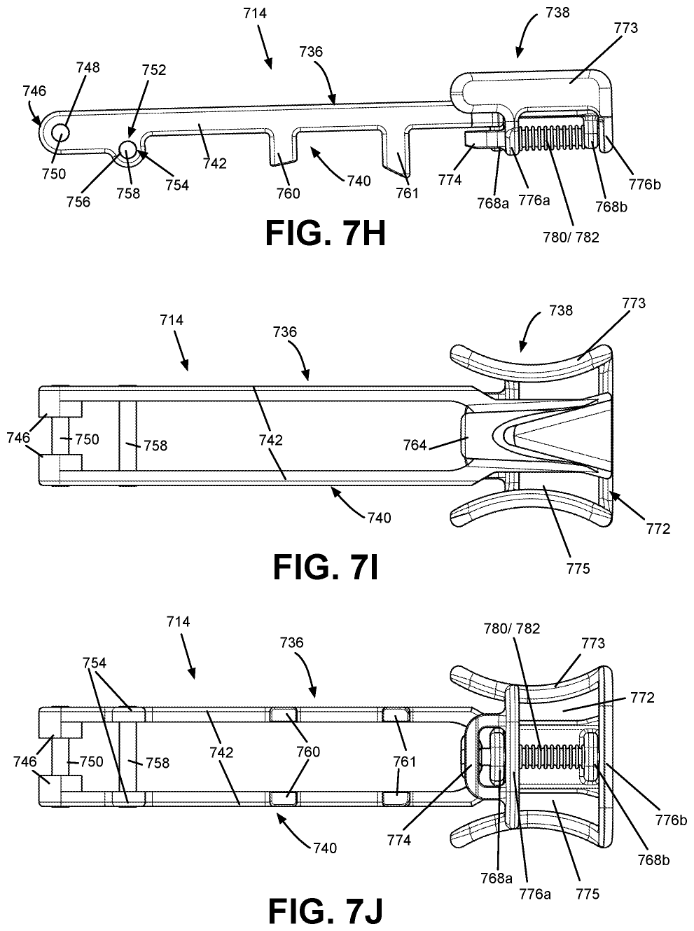

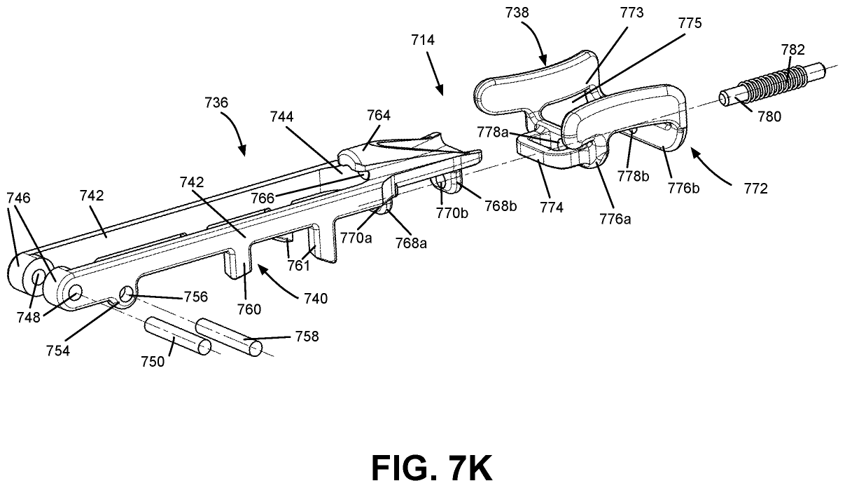

FIGS. 7A and 7B are top and left side views, respectively, illustrating one embodiment of a needle guidance device 710 for use with an ultrasound probe 700 in an assembled or locked configuration, consistent with embodiments described herein. FIG. 7C and 7D are left side plan and right rear isometric views, respectively, illustrating needle guidance device 710 and ultrasound probe 700 in an open or pre-assembled configuration.

Consistent with embodiments described herein, ultrasound probe 700 and needle guidance device 710 are configured to be lockingly coupled to each other so that, during use, needle guidance device 710 is fixed relative to ultrasound probe 700. As shown in FIGS. 7A-7D ultrasound probe 700 includes one or more features that facilitate cooperative engagement with needle guidance device 710. In particular, ultrasound probe 700 includes a generally tubular body 701 having a forward end 702, an intermediate portion, and a rearward end 704.

As shown, forward end 702 includes a generally bulbous portion having a diameter larger than that of the intermediate portion between the forward and rearward ends. Rearward end 704 includes a handle portion 705 and a transition portion 706 to transition a shape of body 701 between handle portion 705 and intermediate portion 703. As shown, transition portion 706 includes a least a portion having a larger diameter than that of intermediate portion 703 and handle portion 705.

Consistent with implementations described herein, and in contrast to previously described embodiments, neither forward end 702, nor transition portion 706 include specific locating features for cooperating with needle guidance device 710. Rather, as described below, features of needle guidance device 710 allow for positive securement of needle guidance device 710 onto ultrasound probe 700 using existing aspects or features of the probe design to prevent rotation and fore and aft movement.

Needle guidance device 710 includes a guide body 712 and a lock assembly 714. Guide body 712 includes a guide tube 716, a forward securing member 718, and a rear securing member 720. FIGS. 7E-7G are left side, top, and cross-sectional views, respectively, of guide body 712. The cross-sectional view of FIG. 7G is taken along the line A-A in FIG. 7F.

As shown in FIGS. 7E-7G, guide tube 716 comprises a generally cylindrical configuration having a central aperture 722 extending therethrough and sized for receiving a needle or other puncture device. In some implementations, guide tubes 716 having different sized apertures 722 may be provided for different gauge puncture devices. In some embodiments, rather than an enclosed central aperture 722, guide tube 716 may include a channel or slot formed vertically therein, into which the puncture device is received.

An intermediate portion of guide tube 716 includes cut out portion 724 that includes a transverse aperture 726 extending therethrough and positioned below the central aperture 722 in guide tube 716, as shown in FIG. 7G. As described below, transverse aperture 726 is sized to receive a pivot pin when assembled together with lock assembly 714. In addition, cut out portion 724 includes a planar upper portion 728, which forms a stop for engaging a portion of lock assembly 714, as also described below.

Consistent with implementations described herein, forward securing member 718 includes a ring configuration 730 that projects downwardly from a forward portion of guide tube 716 and which includes a shape configured to correspond to a shape of forward end 702 of ultrasound probe 700 to capture forward end 702 of ultrasound probe 700 within forward securing member 718 during use.

In this embodiment, rear securing member 720 also includes a ring configuration 732 that projects downwardly from guide tube 716 and which is configured to engage a portion of transition portion 706 of ultrasound probe 700 having its maximum diameter. In addition, as shown in FIG. 7G, rear securing member 720 may be contoured to configure with a surface configuration of transition portion 706 so as to prevent forward longitudinal movement of guide body 712 relative to ultrasound probe 700 once rear securing member 720 is engaged with transition portion 706.

FIGS. 7H-7K are left side, top, bottom, and exploded isometric views, respectively, of lock assembly 714 consistent with implementations described herein. As shown, lock assembly 714 includes a lever component 736 and a latch component 738 which together cooperatively engage guide tube 716 and ultrasound probe 700 to secure needle guidance device 700 to ultrasound probe 700 in the manner described below.

As shown in FIGS. 7I-7K, lever component 736 includes a body portion 740 having a pair of opposing arms 742 and a guide tube alignment portion 744. Arms 742 are sized to accommodate at least a portion of guide tube 716 therebetween, as shown in FIG. 7F. A forward end of each of arms 742 includes an engagement portion 746 for engaging cut out portion 724 in guide tube 716. As shown in FIGS. 7H and 7K, each engagement portion 746 includes an aperture 748 therethrough for aligning with transverse aperture 726 in cut out portion 724. During assembly, apertures 748 and 726 receive a pivot pin 750 therethrough.

Consistent with embodiments described herein, arms 742 also include a pivot limiting element 752 configured to limit the extent to which lever component 736 may be pivoted with respect to guide tube 716. In one implementation, pivot limiting element 752 includes downward projections 754 in arms 742 that include apertures 756 therethrough which may receive a limiting pin 758 therethrough. The position of apertures 756 relative to apertures 748 define the pivot limit for lock assembly 714. In other implementations, pivot limiting element 752 may include a different structure, such as a fixed bar which joins arms 742 in a location similar to limiting pin 758.

As shown in FIGS. 7H and 7K, arms 742 in lever component 736 may also include a pair of forward locating elements 760 and a pair of rearward locating elements 761 which project downwardly therefrom. As shown in FIGS. 7A-7D, forward locating elements 760 and rearward locating elements 761 are positioned on lever component 736 so as to straddle or engage an upper portion of rear securing member 720 when in the locked or closed position. As shown in FIGS. 7C and 7K, lower ends of forward locating elements 760 and rearward locating elements 761 may be contoured to coordinate with an upper surface of ultrasound probe 700. Such a configuration, in combination with forward and rear securing members 718/720, prevents movement of needle guidance device 710 relative to ultrasound probe 700 during use.

As shown, a rearward end of lever component 736 includes a guide tube alignment portion 744 which joins arms 742. As shown in FIG. 7K, guide tube alignment portion 744 includes an upper guide tube engagement feature 764 and a central aperture 766 longitudinally positioned to align with central aperture 722 in guide tube 716, when latch component 738 is engaged with lever component 736 and guide tube 716, as described below. As shown in FIG. 7B, upper guide tube engagement feature 764 includes a portion which projects forwardly relative to a remainder of guide tube alignment portion 744. When lock assembly 714 is latched onto guide tube 716, upper guide tube engagement feature 764 is configured to engage an upper, rear end of guide tube 716 in a position in which aperture 766 is aligned with aperture 722.

As shown in FIGS. 7B, 7H, and 7K, guide tube alignment portion 744 further includes downwardly projecting tabs 768a and 768b. Tabs 768a and 768b include apertures 770a and 770b formed therethrough and function to engage a biasing element in latch component 738, as described below.

As shown in FIGS. 7I-7K and FIG. 7A-7B, latch component 738 comprises a generally frame-like member 772 configured to engage both a rearward portion guide tube 716 of and lever component 736. In particular, as shown, latch component 738 includes a latch engagement portion 773 and a lower guide tube engagement feature 774. Latch engagement portion 773 projects upwardly from frame-like member 772 and provides an engagement surface with which a user may manipulate (i.e., open and close) latch component 738, as described below. Frame-like member 772 include cavity 775 therein for receiving at least a portion of guide tube alignment portion 744, as shown in FIG. 7A. Frame-like member 772 includes a pair of tab engagement elements 776a and 776b that include alignment apertures 778a and 778b formed therethrough. Lower guide tube engagement feature 774 projects forwardly from forward tab engagement element 776a and engages a lower surface of guide tube 716 when latch component 738 is in a latched position with respect to guide tube 716.

During assembly, guide tube alignment portion 744 of lever component 736 is inserted with cavity 775 such that tab engagement elements 776a and 776b abut respective rearward surfaces of tabs 768a and 768b. A rod 780 having a biasing element, such as a spring, 782 positioned thereon is inserted and secured within apertures 770a/770b and 778a/778b, such that biasing element 782 is positioned between a rearward side of forward tab engagement element 776 and a forward side of rearward tab 768b, as shown in FIG. 7J.

To secure needle guidance device 710 to ultrasound probe 700, an operator positions guide tube 716 relative to ultrasound probe 700 such that forward securing member 718 and rearward securing member 720 of guide tube 716 engage forward end 702 and transition portion 706 of ultrasound probe 700, respectively. Guide tube 716 is then fixed relative to ultrasound probe 700 by seating transition portion 706 within rear securing member 720.

Latch component 738 is then opened by pulling rearwardly on latch engagement portion 773, thus compressing biasing element 782 and allowing lower guide tube engagement feature 774 to move rearwardly relative to lever component 736. Lever component 736 is then pivoted about pivot pin 750 such that forward locating elements 760 and rearward locating elements 761 engage rear securing member 720, as described above. Latch engagement portion 773 is then released, which causes biasing element 782 to urge lower guide tube engagement feature 774 to move forwardly relative to lever component 736. In its forward position, lower guide tube engagement feature 774 engages a lower surface of guide tube 716 and upper guide tube engagement feature 764 engages a lower surface of guide tube 716, effectively securing guide tube 716 to ultrasound probe 700.

Consistent with embodiments described herein, central aperture 766 in guide tube alignment portion 744 is only aligned with central aperture 722 in guide tube 716 when guide tube 716 is securely latched onto ultrasound probe, thus preventing premature or unsafe use of needle guidance device 710.

Upon completion of a procedure, needle guidance device 710 may be removed from ultrasound probe 700 by reversing the above steps. That is, latch engagement portion 773 may be again moved rearwardly to release guide tube 716 from lower guide tube engagement feature 774. Lever component 736 may then be pivoted about pivot pin 750 such that forward and rearward locating elements 760/ 761 becomes disengaged from rear securing member 720. At this point, needle guidance device 710 may be removed from ultrasound probe 700 by sliding guide body 712 forward relative to ultrasound probe 700.

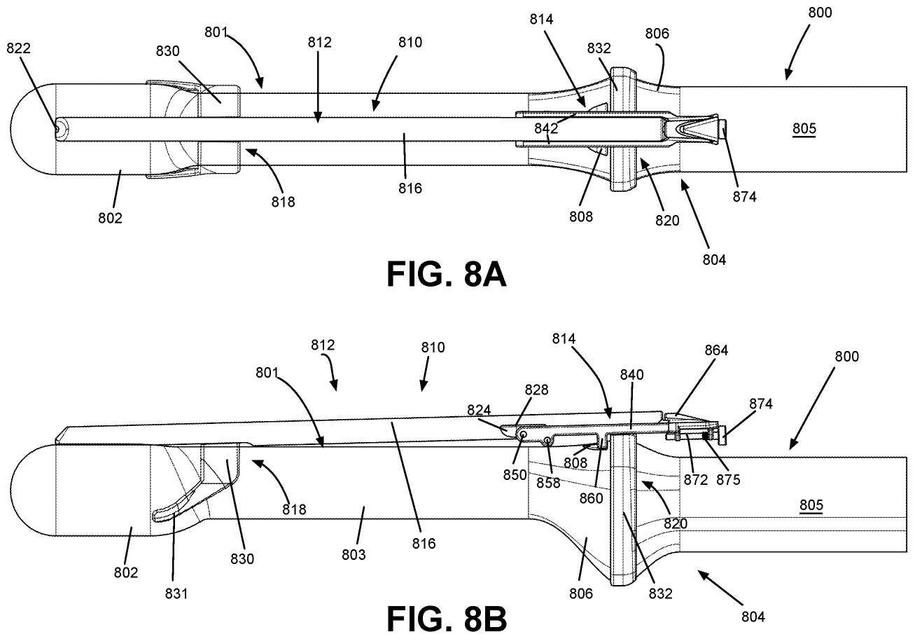

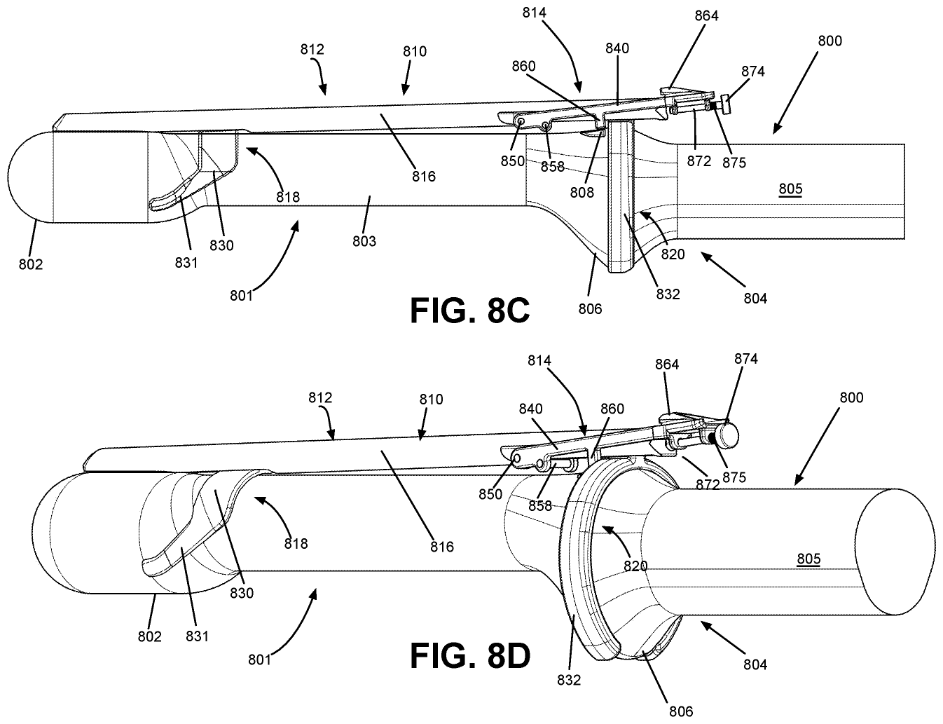

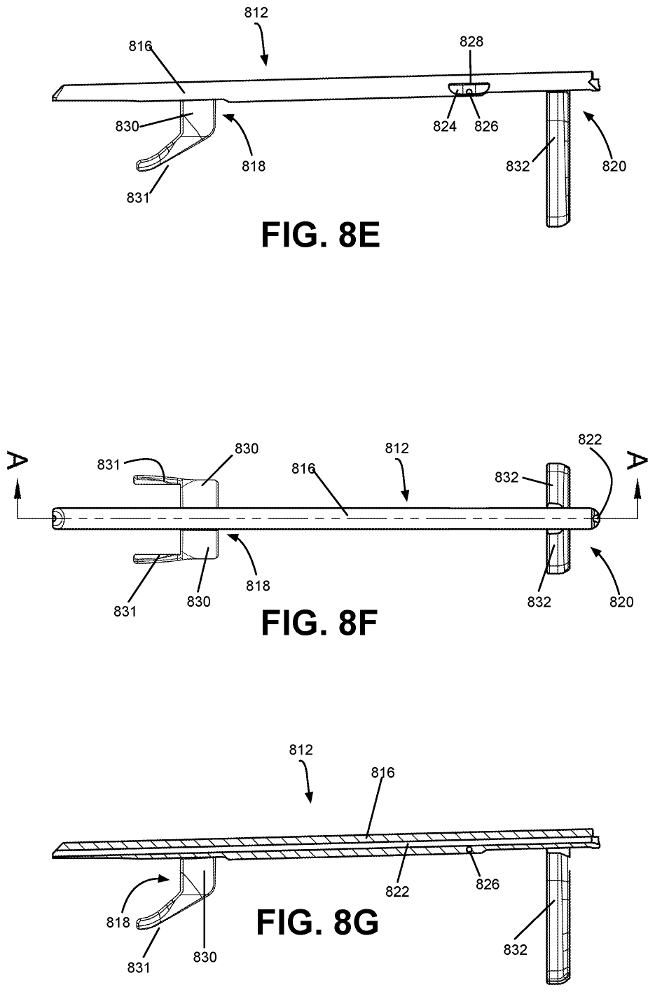

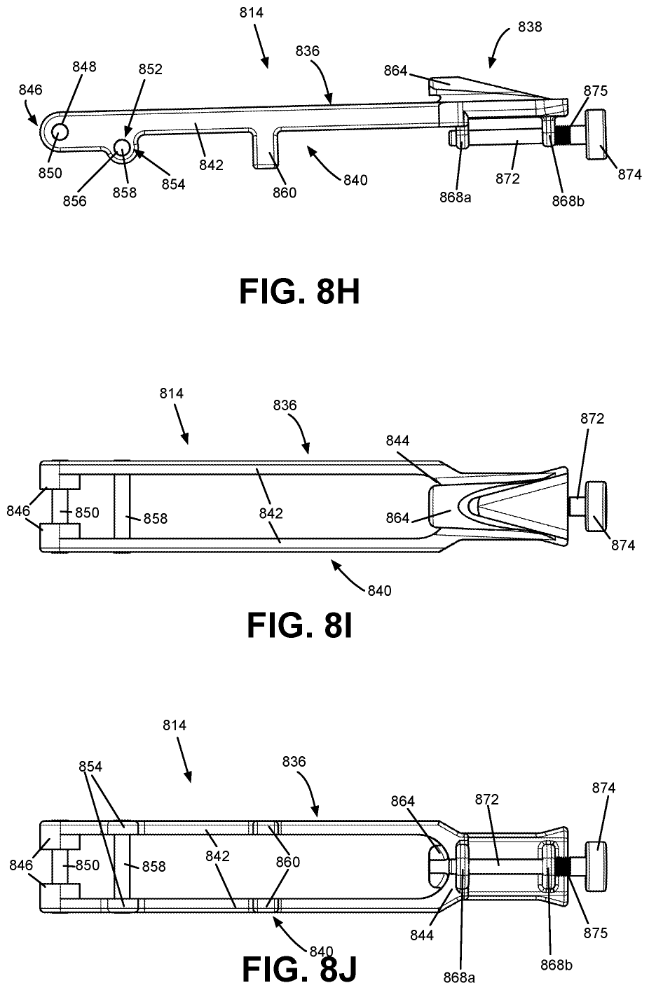

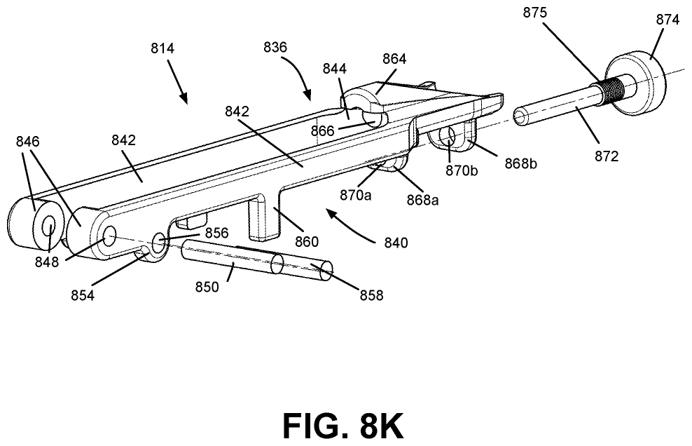

FIGS. 8A and 8B are top and left side views, respectively, illustrating one embodiment of a needle guidance device 810 for use with an ultrasound probe 800 in an assembled or locked configuration, consistent with embodiments described herein. FIG. 8C and 8D are left side plan and right rear isometric views, respectively, illustrating needle guidance device 810 and ultrasound probe 800 in an open or pre-assembled configuration.

Consistent with embodiments described herein, ultrasound probe 800 and needle guidance device 810 are configured to be lockingly coupled to each other so that, during use, needle guidance device 810 is fixed relative to ultrasound probe 800. As shown in FIGS. 8A-8D ultrasound probe 800 includes one or more features that facilitate cooperative engagement with needle guidance device 810. In particular, ultrasound probe 800 includes a generally tubular body 801 having a forward end 802, an intermediate portion 803, and a rearward end 804. As shown, forward end 802 includes a generally bulbous portion having a diameter larger than that of intermediate portion 803. Rearward end 804 includes a handle portion 805 and a transition portion 806 to transition a shape of body 801 between handle portion 805 and intermediate portion 803. As shown, transition portion 806 includes a least a portion having a larger diameter than that of intermediate portion 803 and handle portion 805.

Consistent with implementations described herein, transition portion 806 includes a pair of locating pockets 808 positioned on an upper surface of probe 800 forward of a portion of probe 800 having the largest diameter. Locating pockets 808 include a forward-facing cavity having a rear wall therein. As described in additional detail below, locating pockets 808 facilitate positioning and securing of needle guidance device 810 to ultrasound probe 800 during assembly.

Needle guidance device 810 includes a guide body 812 and a lock assembly 814. Guide body 812 includes a guide tube 816, a forward securing member 818, and a rear securing member 820. FIGS. 8E-8G are left side, top, and cross-sectional views, respectively, of guide body 812. The cross-sectional view of FIG. 8G is taken along the line A-A in FIG. 8F.

As shown in FIGS. 8E-8G, guide tube 816 comprises a generally cylindrical configuration having a central aperture 822 extending therethrough and sized for receiving a needle or other puncture device. In some implementations, guide tubes 816 having different sized apertures 822 may be provided for different gauge puncture devices. In some embodiments, rather than an enclosed central aperture 822, guide tube 816 may include a channel or slot formed vertically therein, into which the puncture device is received.

An intermediate portion of guide tube 816 includes cut out portion 824 that includes a transverse aperture 826 extending therethrough and positioned below the central aperture 822 in guide tube 816, as shown in FIG. 8G. As described below, transverse aperture 826 is sized to receive a pivot pin when assembled together with lock assembly 814. In addition, cut out portion 824 includes a planar upper portion 828, which forms a stop for engaging a portion of lock assembly 814, as also described below.

Forward securing member 818 includes opposing curved arms 830 that project downwardly from a forward portion of guide tube 816 and which include a shape configured to correspond to a shape of forward end 802 of ultrasound probe 800. As shown in FIG. 8B, curved arms 830 may include forward facing prongs 831 contoured to correspond to a shape of the rearward side of forward end 802 to positively position needle guidance device 810 longitudinally relative to ultrasound probe during use, as described below.

In this manner, insertion of forward end 802 of ultrasound probe 800 within curved arms 830 effectively captures an axial position of the forward end of guide tube 816 relative to ultrasound probe 800. That is, the forward end of guide tube 816 may not be pulled radially outwardly from ultrasound probe 800 when forward end 802 of ultrasound probe 800 is positioned with curved arms 830.

Rear securing member 820 also includes curved arms 832 that project downwardly from guide tube 816 and which are configured to engage an outer surface of transition portion 806 of ultrasound probe 800. Consistent with the embodiment of FIGS. 8A-G, curved arms 832 may also be configured to extend more than 180°. In this manner, insertion of rearward end 802 of ultrasound probe 800 within curved arms 832 effectively captures an axial position of the rearward end of guide tube 816 relative to ultrasound probe 800. That is, the rearward end of guide tube 816 may not be pulled radially outwardly from ultrasound probe 800 when transition portion 806 of ultrasound probe 800 is positioned with curved arms 832. In addition, as shown in FIG. 8G, curved arms 832 of rear securing member 820 may be contoured to configure with a surface configuration of transition portion 806 so as to prevent forward longitudinal movement of guide body 812 relative to ultrasound probe 800 once curved arms 832 are engaged with transition portion 806.

FIGS. 8H-8K are left side, top, bottom, and exploded isometric views, respectively, of lock assembly 814 consistent with implementations described herein. As shown, lock assembly 814 includes a lever component 836 and a latch component 838 which together cooperatively engage guide tube 816 and ultrasound probe 800 to secure needle guidance device 800 to ultrasound probe 800 in the manner described below.

As shown in FIGS. 8I-8K, lever component 836 includes a body portion 840 having a pair of opposing arms 842 and a guide tube alignment portion 844. Arms 842 are sized to accommodate at least a portion of guide tube 816 therebetween, as shown in FIG. 8F. A forward end of each of arms 842 includes an engagement portion 846 for engaging cut out portion 824 in guide tube 816. As shown in FIGS. 8H and 8K, each engagement portion 846 includes an aperture 848 therethrough for aligning with transverse aperture 826 in cut out portion 824. During assembly, apertures 848 and 826 receive a pivot pin 850 therethrough.

Consistent with embodiments described herein, arms 842 also include a pivot limiting element 852 configured to limit the extent to which lever component 836 may be pivoted with respect to guide tube 816. In one implementation, pivot limiting element 852 includes downward projections 854 in arms 842 that include apertures 856 therethrough which may receive a limiting pin 858 therethrough. The position of apertures 856 relative to apertures 848 define the pivot limit for lock assembly 814. In other implementations, pivot limiting element 852 may include a different structure, such as a fixed bar which joins arms 842 in a location similar to limiting pin 858.

As shown in FIGS. 8H and 8K, arms 842 in lever component 836 may also include pocket engagement elements 860 which project downwardly therefrom. As shown in FIG. 8A, when locked onto ultrasound probe 800, pocket engagement elements 860 become seated within pockets 808. When pocket engagement elements 860 engage locating pockets 808 and rear securing member 820 positively engages transition portion 806 of ultrasound probe 800, needle guidance device becomes fixed relative to ultrasound probe 800.

As shown, a rearward end of lever component 836 includes a guide tube alignment portion 844 which joins arms 842. As shown in FIG. 8K, guide tube alignment portion 844 includes an upper guide tube engagement feature 864 and a central aperture 866 longitudinally positioned to align with central aperture 822 in guide tube 816, when latch component 838 is engaged with lever component 836 and guide tube 816, as described below. As shown in FIG. 8B, upper guide tube engagement feature 864 includes a portion which projects forwardly relative to a remainder of guide tube alignment portion 844. When lock assembly 814 is latched onto guide tube 816, upper guide tube engagement feature 864 is configured to engage an upper, rear end of guide tube 816 in a position in which aperture 866 is aligned with aperture 822.

As shown in FIGS. 8B, 8H, and 8K, guide tube alignment portion 844 further includes downwardly projecting tabs 868a and 868b. Tabs 868a and 868b include apertures 870a and 870b formed therethrough and function to engage latch component 838, as described below. In one exemplary implementation, one or both of apertures 870a/870b may include a threaded configuration for engaging a threaded portion of latch component 838.

As shown in FIGS. 8H-8K and FIG. 8A-8B, latch component 838 comprises a rod 872 and a latching knob 874. As shown, during use, rod 872 is configured to engage both a rearward portion guide tube 816 of and lever component 836. In particular, as shown, rod 872 is sized for receipt within apertures 870a/870b and may include a threaded portion for 875 for engaging correspondingly threaded portions of apertures 870a/870b. When fully inserted or threaded into apertures 870a/870b, as shown in FIG. 8A, a forward portion of rod 872 engages a lower surface of guide tube 816 to prevent pivoting of guide tube 816 relative to ultrasound probe 800.

To secure needle guidance device 810 to ultrasound probe 800, an operator positions guide tube 816 relative to ultrasound probe 800 such that forward securing member 818 and rearward securing member of guide tube 816 engage forward end 802 and transition portion 806 of ultrasound probe 800, respectively. Guide tube 816 is then fixed relative to ultrasound probe 800 by seating transition portion 806 within curved arms 832 of rear securing member 820.

Lever component 836 is then pivoted about pivot pin 850 such that pocket engagement elements 860 become seated within locating pockets 808. Rod 872 is inserted through apertures 870a/870b, such as by turning knob 874. In this manner, the forward end of rod 872 is brought into engagement with a lower surface of guide tube 816 and upper guide tube engagement feature 864 engages a lower surface of guide tube 816, effectively securing guide tube 816 to ultrasound probe 800.

Consistent with embodiments described herein, central aperture 866 in guide tube alignment portion 844 is only aligned with central aperture 822 in guide tube 816 when guide tube 816 is securely latched onto ultrasound probe, thus preventing premature or unsafe use of needle guidance device 810.

Upon completion of a procedure, needle guidance device 810 may be removed from ultrasound probe 800 by reversing the above steps. That is, rod 872 may be moved (e.g., threadingly) rearwardly to release guide tube 816 from the forward end of rod 872. Lever component 836 may then be pivoted about pivot pin 850 such that pocket engagement elements 860 becomes disengaged from locating pockets 808. At this point, needle guidance device 810 may be removed from ultrasound probe 800 by sliding guide body 812 forward relative to ultrasound probe 800.

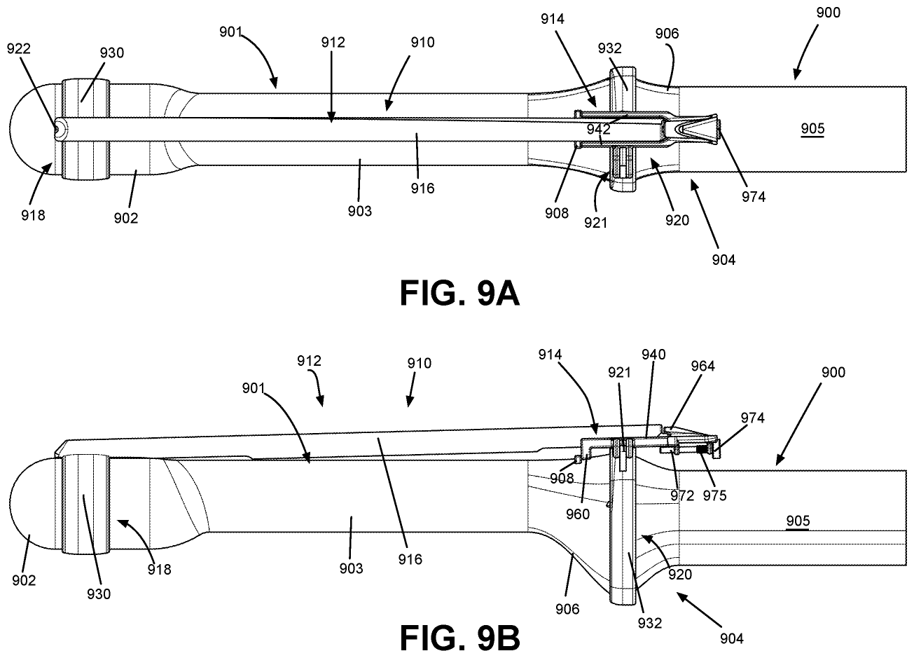

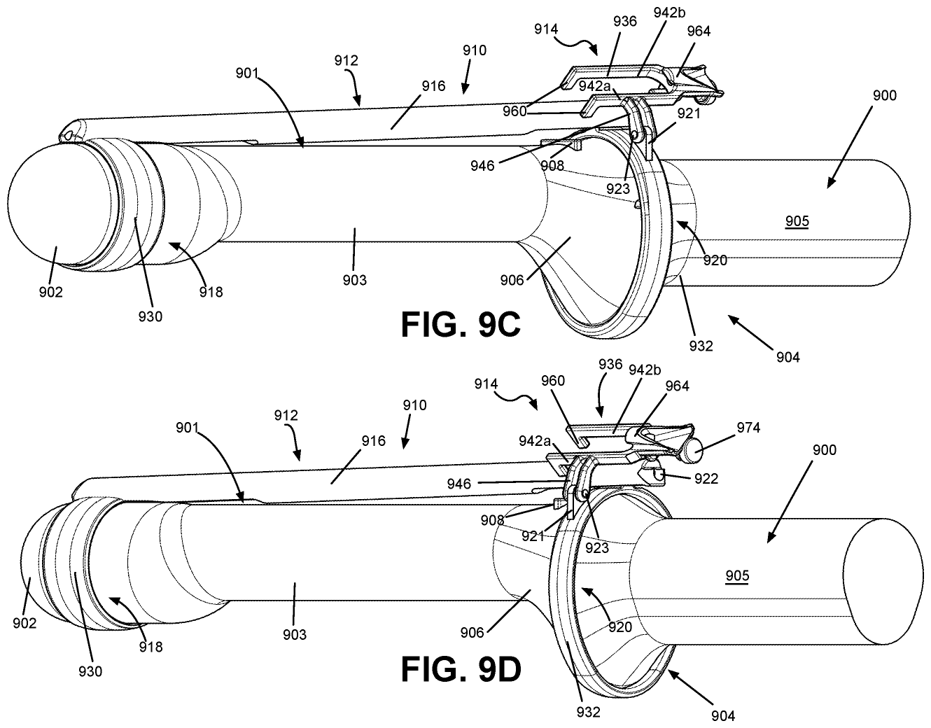

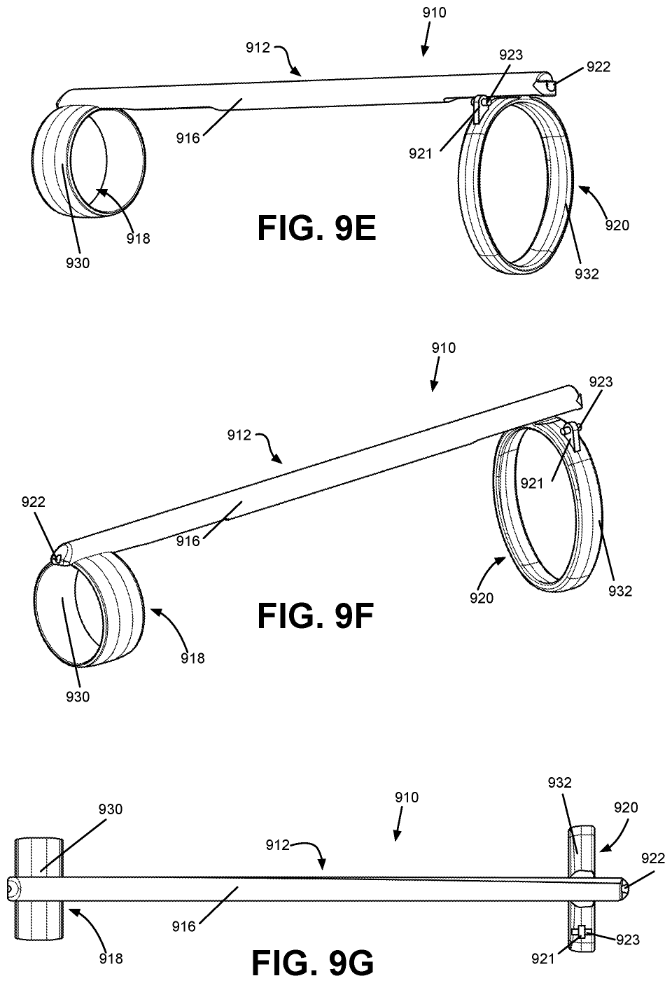

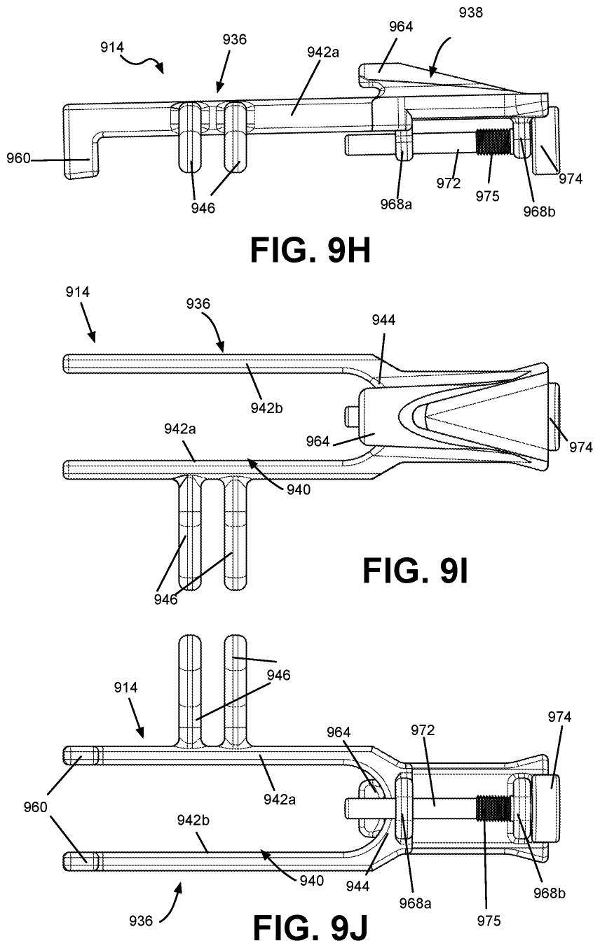

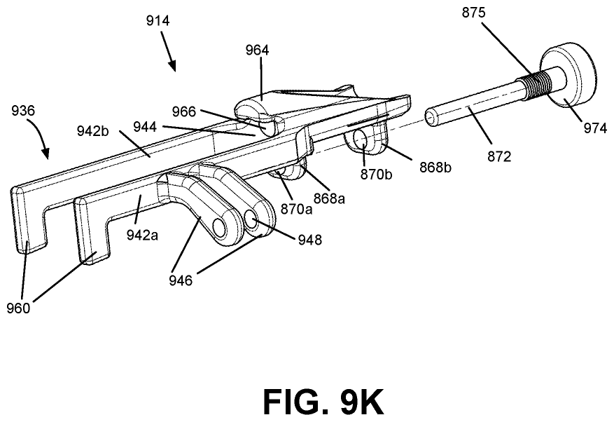

FIGS. 9A and 9B are top and left side views, respectively, illustrating one embodiment of a needle guidance device 910 for use with an ultrasound probe 900 in an assembled or locked configuration, consistent with embodiments described herein. FIG. 9C and 9D are left side plan and right rear isometric views, respectively, illustrating needle guidance device 910 and ultrasound probe 900 in an open or pre-assembled configuration.

Consistent with embodiments described herein, ultrasound probe 900 and needle guidance device 910 are configured to be lockingly coupled to each other so that, during use, needle guidance device 910 is fixed relative to ultrasound probe 900. As shown in FIGS. 9A-9D ultrasound probe 900 includes one or more features that facilitate cooperative engagement with needle guidance device 910. In particular, ultrasound probe 900 includes a generally tubular body 901 having a forward end 902, an intermediate portion 903, and a rearward end 904. As shown, forward end 902 includes a generally bulbous portion having a diameter larger than that of intermediate portion 903. Rearward end 904 includes a handle portion 905 and a transition portion 906 to transition a shape of body 901 between handle portion 905 and intermediate portion 903. As shown, transition portion 906 includes a least a portion having a larger diameter than that of intermediate portion 903 and handle portion 905.

Consistent with implementations described herein, transition portion 906 includes a locating rib 908 projecting from a top surface of probe 900 for engaging with a corresponding feature in needle guidance device 910, as described below.

Needle guidance device 910 includes a guide body 912 and a lock assembly 914. Guide body 912 includes a guide tube 916, a forward securing member 918, and a rear securing member 920. FIGS. 9E-9G are left side, top, and cross-sectional views, respectively, of guide body 912. The cross-sectional view of FIG. 9G is taken along the line A-A in FIG. 9F.

As shown in FIGS. 9E-9G, guide tube 916 comprises a generally cylindrical configuration having a central aperture 922 extending therethrough and sized for receiving a needle or other puncture device. In some implementations, guide tubes 916 having different sized apertures 922 may be provided for different gauge puncture devices. In some embodiments, rather than an enclosed central aperture 922, guide tube 916 may include a channel or slot formed vertically therein, into which the puncture device is received.

Consistent with implementations described herein, forward securing member 918 includes a ring configuration 930 that projects downwardly from a forward portion of guide tube 916 and which includes a shape configured to correspond to a shape of forward end 902 of ultrasound probe 900 to capture forward end 902 of ultrasound probe 900 within forward securing member 918 during use.

In this manner, insertion of forward end 902 of ultrasound probe 900 within forward securing member 918 effectively captures an axial position of the forward end of guide tube 916 relative to ultrasound probe 900. That is, the forward end of guide tube 916 may not be pulled radially outwardly from ultrasound probe 900 when forward end 902 of ultrasound probe 900 is positioned within forward securing member 918.

Rear securing member 920 also includes a ring configuration 930 that projects downwardly from guide tube 916 and which is configured to engage a portion of transition portion 906 of ultrasound probe 900 having its maximum diameter. In addition, as shown in FIG. 9G, rear securing member 920 may be contoured to configure with a surface configuration of transition portion 906 so as to prevent forward longitudinal movement of guide body 912 relative to ultrasound probe 900 once rear securing member 920 is engaged with transition portion 906.

As shown in FIG. 9E-9G, a portion of rear securing member 920 includes a lock assembly engagement feature 921 for pivotably connecting to lock assembly 914, as described below. In one exemplary implementation, lock assembly engagement feature 921 projects upwardly from an upper portion of rear securing member 920 at a position laterally offset from guide tube 916. As shown, lock assembly engagement feature 921 includes a hinge pin 923 for engagement with lock assembly 914.

FIGS. 9H-9K are left side, top, bottom, and exploded isometric views, respectively, of lock assembly 914 consistent with implementations described herein. As shown, lock assembly 914 includes a lever component 936 and a latch component 938 which together cooperatively engage guide tube 916 and ultrasound probe 900 to secure needle guidance device 900 to ultrasound probe 900 in the manner described below.

As shown in FIGS. 9I-9K, lever component 936 includes a body portion 940 having a pair of opposing arms 942a and 942b and a guide tube alignment portion 944. Arms 942a and 942b are sized to accommodate at least a portion of guide tube 916 therebetween, as shown in FIG. 9A. In one implementation, an intermediate portion of arm 942a includes an engagement portion 946 for pivotally coupling to lock assembly engagement feature 921. As shown in FIGS. 9H and 9K, engagement portion 946 projects outwardly relative to arm 942a and includes one or more apertures 948 therethrough for aligning with hinge pin 923. During assembly, apertures 948 are coupled to hinge pin 923, such that lever component 936 is able to pivot into and out of engagement with guide tube 916.

As shown in FIGS. 9H and 9K, arms 942 a forward portion of arms 942a and 942b include locating rib engagement elements 960 which project downwardly therefrom. As shown in FIG. 9A, when locked onto ultrasound probe 900, rib engagement elements 960 engage a rearward side of locating rib 908. When rib engagement elements 960 engage locating rib 908 and rear securing member 920 positively engages transition portion 906 of ultrasound probe 900, needle guidance device becomes fixed relative to ultrasound probe 900.

As shown, a rearward end of lever component 936 includes a guide tube alignment portion 944 which joins arms 942. As shown in FIG. 9K, guide tube alignment portion 944 includes an upper guide tube engagement feature 964 and a central aperture 966 longitudinally positioned to align with central aperture 922 in guide tube 916, when latch component 938 is engaged with lever component 936 and guide tube 916, as described below. As shown in FIG. 9B, upper guide tube engagement feature 964 includes a portion which projects forwardly relative to a remainder of guide tube alignment portion 1. When lock assembly 914 is latched onto guide tube 916, upper guide tube engagement feature 964 is configured to engage an upper, rear end of guide tube 916 in a position in which aperture 966 is aligned with aperture 922.

As shown in FIGS. 9B, 9H, and 9K, guide tube alignment portion 944 further includes downwardly projecting tabs 968a and 968b. Tabs 968a and 968b include apertures 970a and 970b formed therethrough and function to engage latch component 938, as described below. In one exemplary implementation, one or both of apertures 970a/970b may include a threaded configuration for engaging a threaded portion of latch component 938.

As shown in FIGS. 9H-9K and FIG. 9A-9B, latch component 938 comprises a rod 972 and a latching knob 974. As shown, during use, rod 972 is configured to engage both a rearward portion guide tube 916 of and lever component 936. In particular, as shown, rod 972 is sized for receipt within apertures 970a/970b and may include a threaded portion for engaging correspondingly threaded portions of apertures 970a/970b. When fully inserted or threaded into apertures 970a/970b, as shown in FIG. 9A, a forward portion of rod 972 engages a lower surface of guide tube 916 to prevent pivoting of guide tube 916 relative to ultrasound probe 900.

To secure needle guidance device 910 to ultrasound probe 900, an operator positions guide tube 916 relative to ultrasound probe 900 such that forward securing member 918 and rearward securing member 920 of guide tube 916 engage forward end 902 and transition portion 906 of ultrasound probe 900, respectively. Guide tube 916 is then fixed relative to ultrasound probe 900 by seating transition portion 906 within curved arms 932 of rear securing member 920.

Lever component 936 is then pivoted about hinge pin 923 such that rib engagement elements 960 become seated behind locating rib 908. Rod 972 is inserted through apertures 970a/970b, such as by turning knob 974. In this manner, the forward end of rod 972 is brought into engagement with a lower surface of guide tube 916 and upper guide tube engagement feature 964 engages a lower surface of guide tube 916, effectively securing guide tube 916 to ultrasound probe 900.

Consistent with embodiments described herein, central aperture 966 in guide tube alignment portion 944 is only aligned with central aperture 922 in guide tube 916 when guide tube 916 is securely latched onto ultrasound probe, thus preventing premature or unsafe use of needle guidance device 910.

Upon completion of a procedure, needle guidance device 910 may be removed from ultrasound probe 900 by reversing the above steps. That is, rod 972 may be moved (e.g., threadingly) rearwardly to release guide tube 916 from the forward end of rod 972. Lever component 936 may then be pivoted about hinge pin 923 such that rib engagement elements 960 become disengaged from locating rib 908. At this point, needle guidance device 910 may be removed from ultrasound probe 900 by sliding guide body 1 forward relative to ultrasound probe 900.

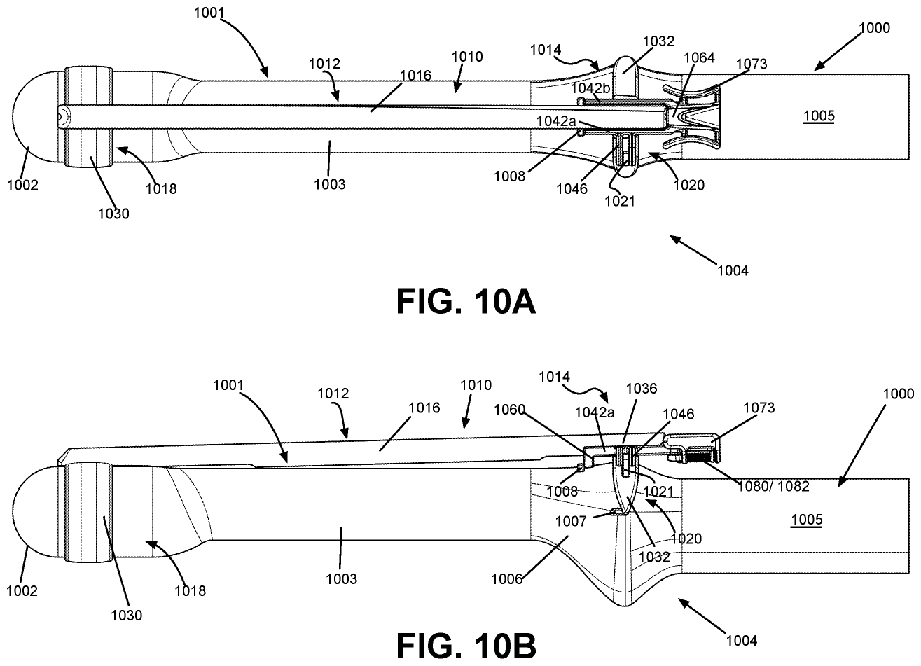

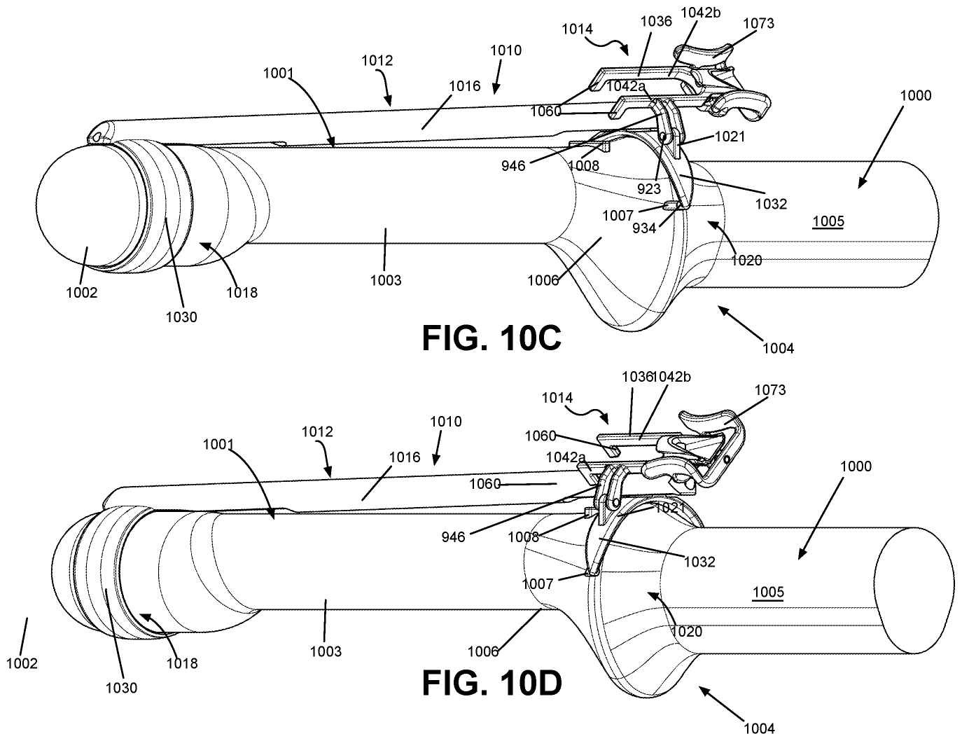

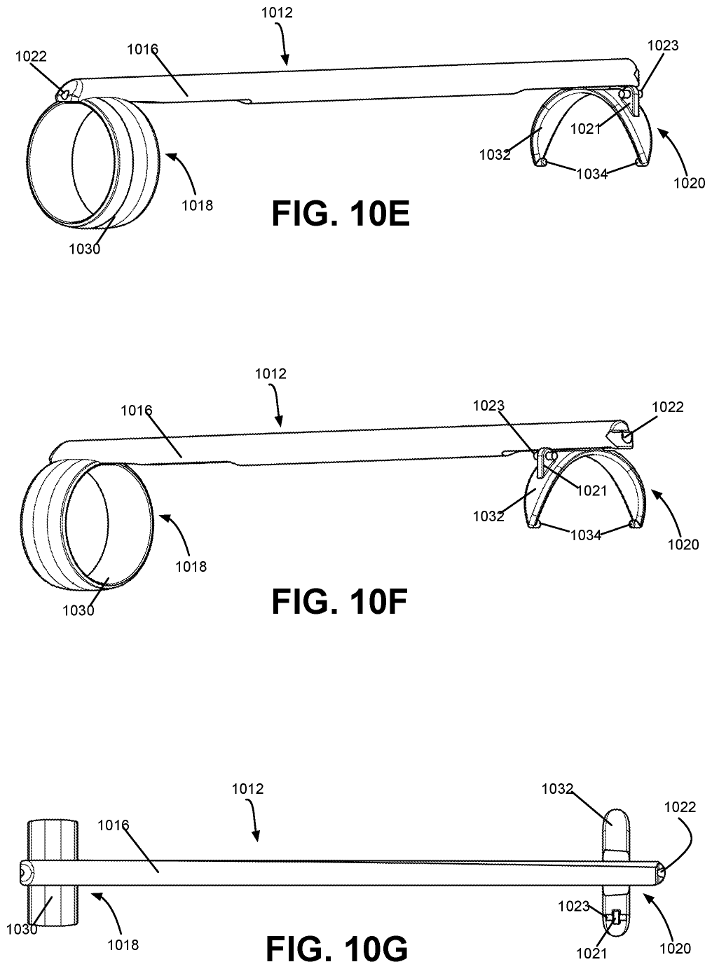

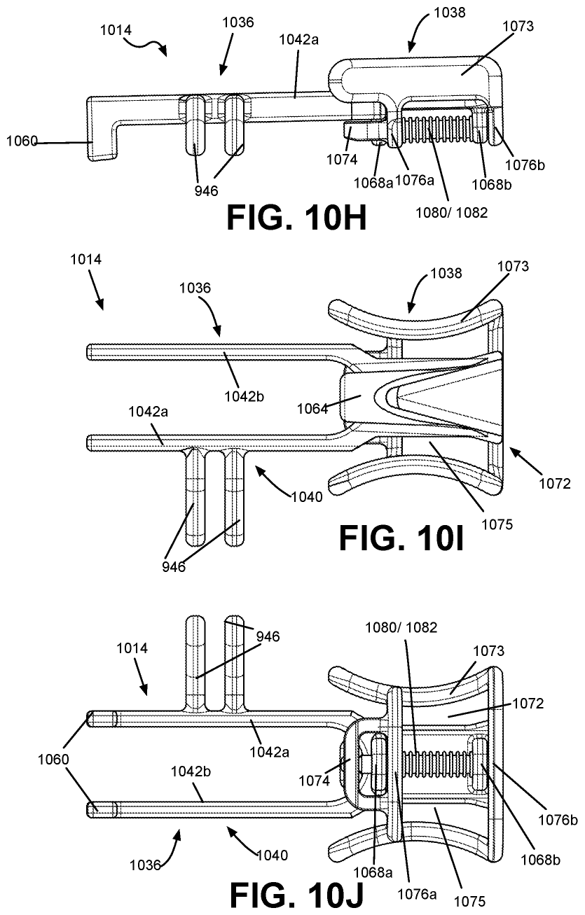

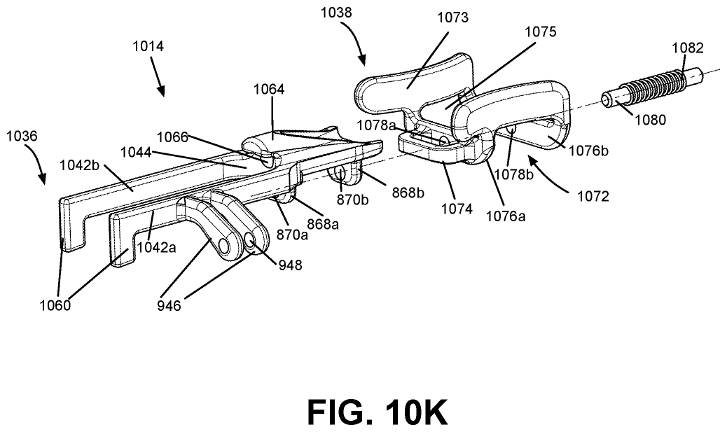

FIGS. 10A and 10B are top and left side views, respectively, illustrating one embodiment of a needle guidance device 1010 for use with an ultrasound probe 1000 in an assembled or locked configuration, consistent with embodiments described herein. FIG. 10C and are left side plan and right rear isometric views, respectively, illustrating needle guidance device 1010 and ultrasound probe 1000 in an open or pre-assembled configuration.

Consistent with embodiments described herein, ultrasound probe 1000 and needle guidance device 1010 are configured to be lockingly coupled to each other so that, during use, needle guidance device 1010 is fixed relative to ultrasound probe 1000. As shown in FIGS. 10A-10D ultrasound probe 1000 includes one or more features that facilitate cooperative engagement with needle guidance device 1010. In particular, ultrasound probe 1000 includes a generally tubular body 1001 having a forward end 1002, an intermediate portion 1003, and a rearward end 1004. As shown, forward end 1002 includes a generally bulbous portion having a diameter larger than that of intermediate portion 1003. Rearward end 1004 includes a handle portion 1005 and a transition portion 1006 to transition a shape of body 1001 between handle portion 1005 and intermediate portion 1003. As shown, transition portion 1006 includes a least a portion having a larger diameter than that of intermediate portion 1003 and handle portion 1005.

Consistent with implementations described herein, transition portion 1006 includes a pair of locating pockets 1007 positioned on opposite sides of probe 1000 and a locating rib 1008 projecting from a top surface of probe 1000. Locating pockets 1007 include a forward-facing cavity having a rear wall therein. As described in additional detail below, locating pockets 1007 and locating rib 1008 collectively facilitate positioning and securing of needle guidance device 1010 to ultrasound probe 1000 during assembly.

Needle guidance device 1010 includes a guide body 1012 and a lock assembly 1014. Guide body 1012 includes a guide tube 1016, a forward securing member 1018, and a rear securing member 1020. FIGS. 10E-10G are left side, top, and cross-sectional views, respectively, of guide body 1012. The cross-sectional view of FIG. 10G is taken along the line A-A in FIG. 10F.

As shown in FIGS. 10E-10G, guide tube 1016 comprises a generally cylindrical configuration having a central aperture 1022 extending therethrough and sized for receiving a needle or other puncture device. In some implementations, guide tubes 1016 having different sized apertures 1022 may be provided for different gauge puncture devices. In some embodiments, rather than an enclosed central aperture 1022, guide tube 1016 may include a channel or slot formed vertically therein, into which the puncture device is received.

Consistent with implementations described herein, forward securing member 1018 includes a ring configuration 1030 that projects downwardly from a forward portion of guide tube 1016 and which includes a shape configured to correspond to a shape of forward end 1002 of ultrasound probe 1000 to capture forward end 1002 of ultrasound probe 1000 within forward securing member 1018 during use.

In this manner, insertion of forward end 1002 of ultrasound probe 1000 within forward securing member 1018 effectively captures an axial position of the forward end of guide tube 1016 relative to ultrasound probe 1000. That is, the forward end of guide tube 1016 may not be pulled radially outwardly from ultrasound probe 1000 when forward end 1002 of ultrasound probe 1000 is positioned within forward securing member 1018.

Rear securing member 1020 includes curved arms 1032 that project downwardly from guide tube 1016 and which are configured to engage an outer surface of transition portion 1006 of ultrasound probe 1000. Consistent with some implementations described herein, curved arms 1032 in rear securing member 1020 include locating elements 1034 which project inwardly at a terminus of curved arms 1032. As shown in FIG. 10A, when assembled, locating elements 1034 are slidingly positioned within locating pockets 1007 in transition portion 1006 of ultrasound probe 1000.

As shown in FIG. 10E-10G, a portion of rear securing member 1020 includes a lock assembly engagement feature 1021 for pivotably connecting to lock assembly 1014, as described below. In one exemplary implementation, lock assembly engagement feature 1021 projects upwardly from an upper portion of rear securing member 1020 at a position laterally offset from guide tube 1016. As shown, lock assembly engagement feature 1021 includes a hinge pin 1023 for engagement with lock assembly 1014.

FIGS. 10H-10K are left side, top, bottom, and exploded isometric views, respectively, of lock assembly 1014 consistent with implementations described herein. As shown, lock assembly 1014 includes a lever component 1036 and a latch component 1038 which together cooperatively engage guide tube 1016 and ultrasound probe 1000 to secure needle guidance device 1000 to ultrasound probe 1000 in the manner described below.

As shown in FIGS. 10I-10K, lever component 1036 includes a body portion 1040 having a pair of opposing arms 1042a and 1042b and a guide tube alignment portion 1044. Arms 1042a and 1042b are sized to accommodate at least a portion of guide tube 1016 therebetween, as shown in FIG. 10A. In one implementation, an intermediate portion of arm 1042a includes an engagement portion 1046 for pivotally coupling to lock assembly engagement feature 1021. As shown in FIGS. 10H and 10K, engagement portion 1046 projects outwardly relative to arm 1042a and includes one or more apertures 1048 therethrough for aligning with hinge pin 1023. During assembly, apertures 1048 are coupled to hinge pin 1023, such that lever component 1036 is able to pivot into and out of engagement with guide tube 1016.

As shown in FIGS. 10H and 10K, arms 1042 a forward portion of arms 1042a and 1042b include locating rib engagement elements 1060 which project downwardly therefrom. As shown in FIG. 10A, when locked onto ultrasound probe 1000, rib engagement elements 1060 engage a rearward side of locating rib 1008. When rib engagement elements 1060 engage locating rib 1008 and rear securing member 1020 positively engages transition portion 1006 of ultrasound probe 1000, needle guidance device becomes fixed relative to ultrasound probe 1000.

As shown, a rearward end of lever component 1036 includes a guide tube alignment portion 1044 which joins arms 1042. As shown in FIG. 10K, guide tube alignment portion 1044 includes an upper guide tube engagement feature 1064 and a central aperture 1066 longitudinally positioned to align with central aperture 1022 in guide tube 1016, when latch component 1038 is engaged with lever component 1036 and guide tube 1016, as described below. As shown in FIG. 10B, upper guide tube engagement feature 1064 includes a portion which projects forwardly relative to a remainder of guide tube alignment portion 1044. When lock assembly 1014 is latched onto guide tube 1016, upper guide tube engagement feature 1064 is configured to engage an upper, rear end of guide tube 1016 in a position in which aperture 1066 is aligned with aperture 1022.

As shown in FIGS. 10B, 10H, and 10K, guide tube alignment portion 1044 further includes downwardly projecting tabs 1068a and 1068b. Tabs 1068a and 1068b include apertures 1070a and 1070b formed therethrough and function to engage latch component 1038, as described below. In one exemplary implementation, one or both of apertures 1070a/1070b may include a threaded configuration for engaging a threaded portion of latch component 1038.

As shown in FIGS. 10I-10K and FIG. 10A-10B, latch component 1038 comprises a generally frame-like member 1072 configured to engage both a rearward portion of guide tube 1016 of and lever component 1036. In particular, as shown, latch component 1038 includes a latch engagement portion 1073 and a lower guide tube engagement feature 1074. Latch engagement portion 1073 projects upwardly from frame-like member 1072 and provides an engagement surface with which a user may manipulate (i.e., open and close) latch component 1038, as described below. Frame-like member 1072 include cavity 1075 therein for receiving at least a portion of guide tube alignment portion 1044, as shown in FIG. 10A. Frame-like member 1072 includes a pair of tab engagement elements 1076a and 1076b that include alignment apertures 1078a and 1078b formed therethrough. Lower guide tube engagement feature 1074 projects forwardly from forward tab engagement element 1076a and engages a lower surface of guide tube 1016 when latch component 1038 is in a latched position with respect to guide tube 1016.

During assembly, guide tube alignment portion 1044 of lever component 1036 is inserted with cavity 1075 such that tab engagement elements 1076a and 1076b abut respective rearward surfaces of tabs 1068a and 1068b. A rod 1080 having a biasing element, such as a spring 1082 is positioned thereon is inserted and secured within apertures 1070a/1070b and 1078a/1078b, such that biasing element 1082 is positioned between a rearward side of forward tab engagement element 1076 and a forward side of rearward tab 1068b, as shown in FIG. 10J.

To secure needle guidance device 1010 to ultrasound probe 1000, an operator positions guide tube 1016 relative to ultrasound probe 1000 such that forward securing member 1018 and rearward securing member 1020 of guide tube 1016 engage forward end 1002 and transition portion 1006 of ultrasound probe 1000, respectively. Guide tube 1016 is then fixed relative to ultrasound probe 1000 by seating locating elements 1034 within locating pockets 1007.

Latch component 1038 is then opened by pulling rearwardly on latch engagement portion 1073, thus compressing biasing element 1082 and allowing lower guide tube engagement feature 1074 to move rearwardly relative to lever component 1036. Latch component 1038 is then pivoted downward about hinge pin 1023 such that rib engagement elements 1068 engage a rearward side of locating rib 1008. Latch engagement portion 1073 is then released, which causes biasing element 1082 to urge lower guide tube engagement feature 174 to move forwardly relative to lever component 1036. In its forward position, lower guide tube engagement feature 1074 engages a lower surface of guide tube 1016 and upper guide tube engagement feature 1064 engages an upper surface of guide tube 1016, effectively securing guide tube 1016 to ultrasound probe 1000.

Consistent with embodiments described herein, central aperture 1066 in guide tube alignment portion 1044 is only aligned with central aperture 1022 in guide tube 1016 when guide tube 1016 is securely latched onto ultrasound probe, thus preventing premature or unsafe use of needle guidance device 1010.

Upon completion of a procedure, needle guidance device 1010 may be removed from ultrasound probe 1000 by reversing the above steps. That is, latch engagement portion 1073 may be again moved rearwardly to release guide tube 1016 from lower guide tube engagement feature 1074. Lever component 1036 may then be pivoted about hinge pin 1023 such that rib engagement elements 1068 becomes disengaged from locating rib 1008. At this point, needle guidance device 1010 may be removed from ultrasound probe 1000 by removing locating elements 1034 from locating pockets 1007.

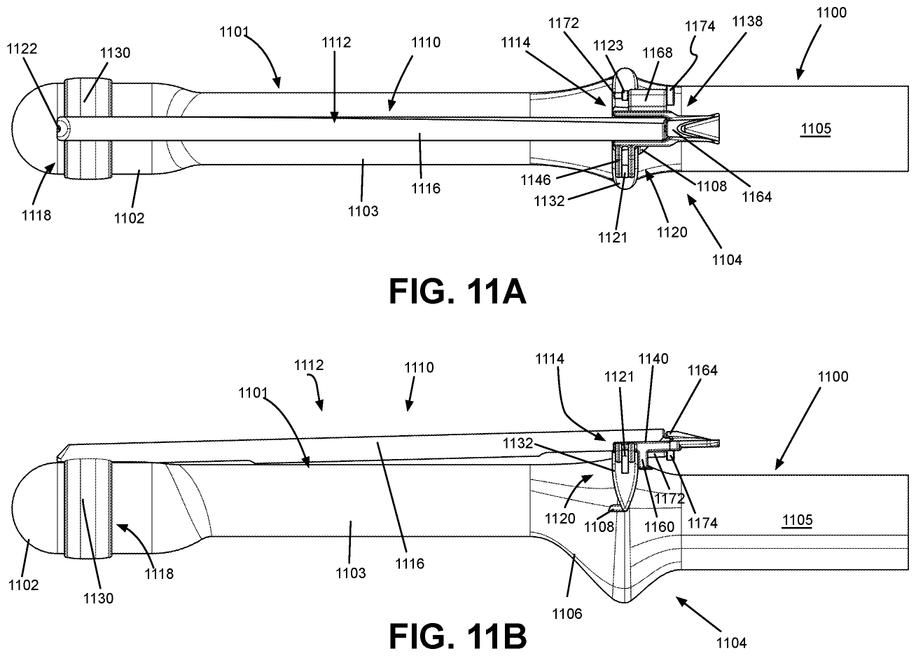

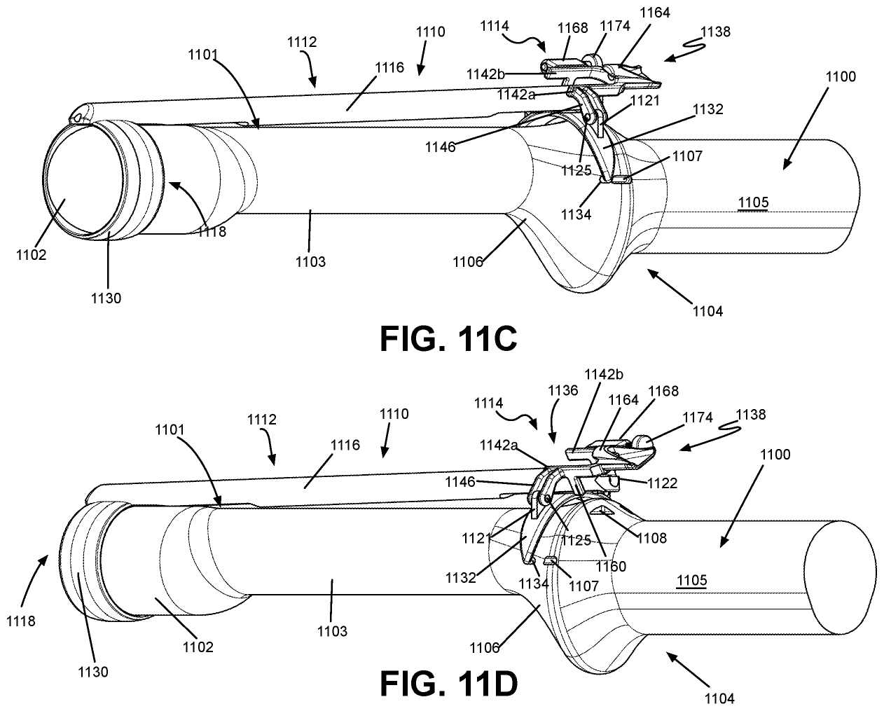

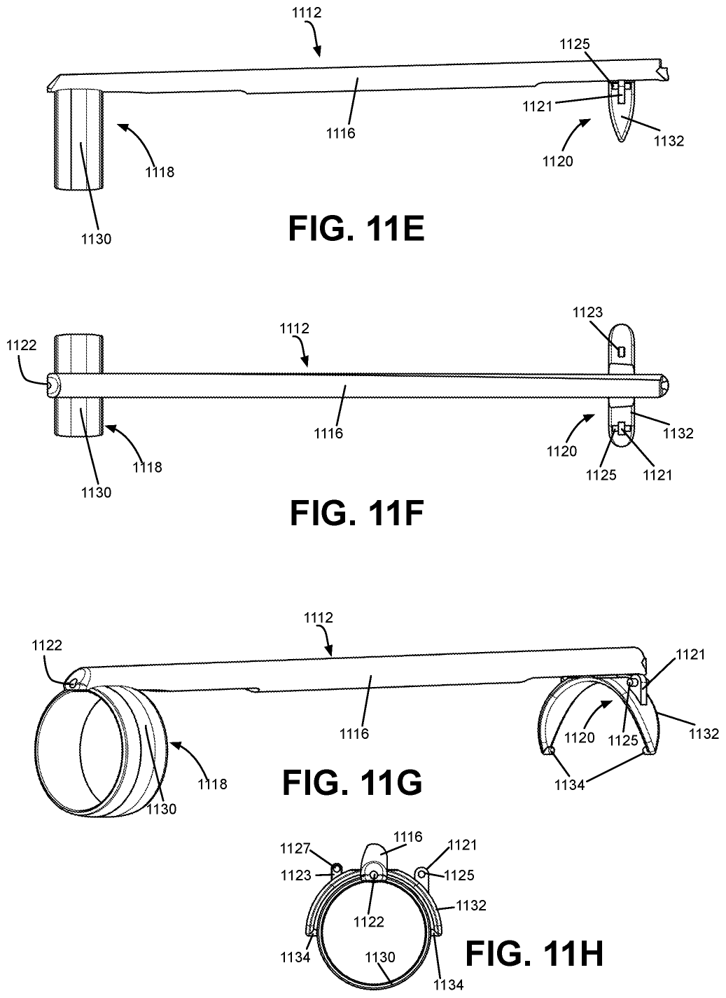

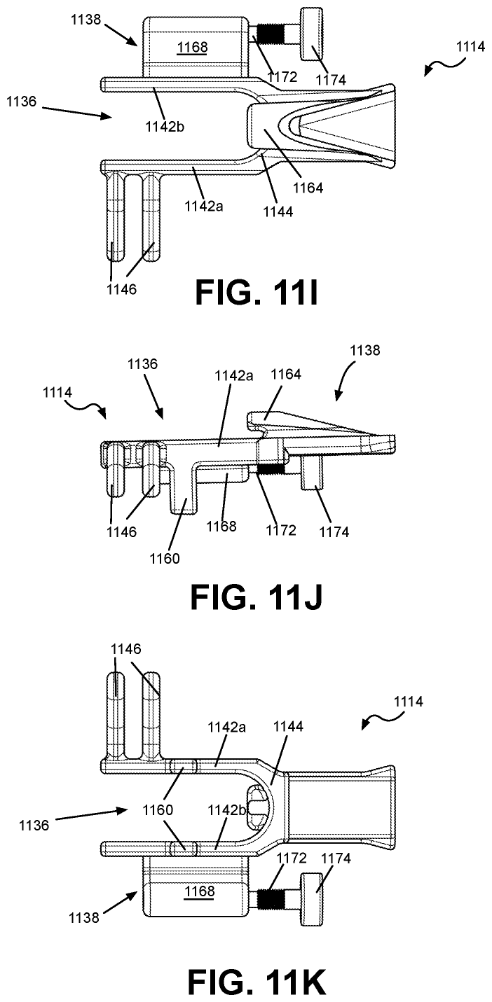

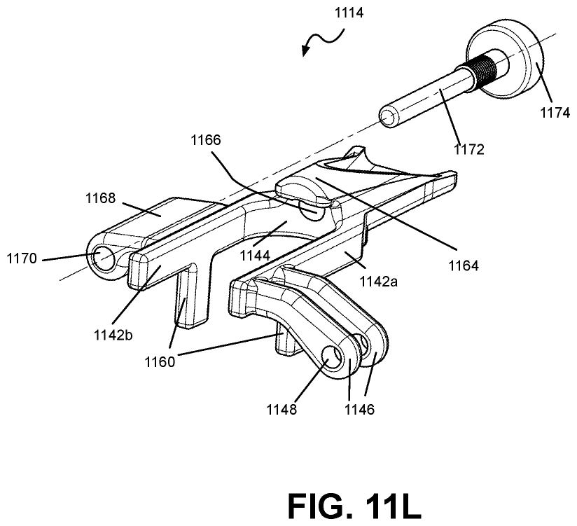

FIGS. 11A and 11B are top and left side views, respectively, illustrating one embodiment of a needle guidance device 1110 for use with an ultrasound probe 1100 in an assembled or locked configuration, consistent with embodiments described herein. FIG. 11C and 11D are left side plan and right rear isometric views, respectively, illustrating needle guidance device 1110 and ultrasound probe 1100 in an open or pre-assembled configuration.

Consistent with embodiments described herein, ultrasound probe 1100 and needle guidance device 1110 are configured to be lockingly coupled to each other so that, during use, needle guidance device 1110 is fixed relative to ultrasound probe 1100. As shown in FIGS. 11A-11D ultrasound probe 1100 includes one or more features that facilitate cooperative engagement with needle guidance device 1110. In particular, ultrasound probe 1100 includes a generally tubular body 1101 having a forward end 1102, an intermediate portion 1103, and a rearward end 1104. As shown, forward end 1102 includes a generally bulbous portion having a diameter larger than that of intermediate portion 1103. Rearward end 1104 includes a handle portion 1105 and a transition portion 1106 to transition a shape of body 1101 between handle portion 1105 and intermediate portion 1103. As shown, transition portion 1106 includes a least a portion having a larger diameter than that of intermediate portion 1103 and handle portion 1105.

Consistent with implementations described herein, transition portion 1106 includes a pair of side locating pockets 1107 positioned on opposite sides of probe 1100 and a pair of upper locating pockets 1108 formed within a top surface of probe 1100. Side locating pockets 1107 each include a rearward-facing cavity having a forward wall therein. As described in additional detail below, side locating pockets 1107 and upper locating pockets 1108 collectively facilitate positioning and securing of needle guidance device 1110 to ultrasound probe 1100 during assembly.

Needle guidance device 1110 includes a guide body 1112 and a lock assembly 1114. Guide body 1112 includes a guide tube 1116, a forward securing member 1118, and a rear securing member 1120. FIGS. 11E-11H are left side, top, front isometric, and front views, respectively, of guide body 1112.

As shown in FIGS. 11E-11H, guide tube 1116 comprises a generally cylindrical configuration having a central aperture 1122 extending therethrough and sized for receiving a needle or other puncture device. In some implementations, guide tubes 1116 having different sized apertures 1122 may be provided for different gauge puncture devices. In some embodiments, rather than an enclosed central aperture 1122, guide tube 1116 may include a channel or slot formed vertically therein, into which the puncture device is received.

Consistent with implementations described herein, forward securing member 1118 includes a ring configuration that projects downwardly from a forward portion of guide tube 1116 and which includes a shape configured to correspond to a shape of forward end 1102 of ultrasound probe 1100 to capture forward end 1102 of ultrasound probe 1100 within forward securing member 1118 during use.

In this manner, insertion of forward end 1102 of ultrasound probe 1100 within forward securing member 1118 effectively captures an axial position of the forward end of guide tube 1116 relative to ultrasound probe 1100. That is, the forward end of guide tube 1116 may not be pulled radially outwardly from ultrasound probe 1100 when forward end 1102 of ultrasound probe 1100 is positioned within forward securing member 1118.

Rear securing member 1120 includes curved arms 1132 that project downwardly from guide tube 1116 and which are configured to engage an outer surface of transition portion 1106 of ultrasound probe 1100. Consistent with some implementations described herein, curved arms 1132 in rear securing member 1120 include locating elements 1134 which project inwardly at a terminus of curved arms 1132. As shown in FIG. 11A, when assembled, locating elements 1134 are slidingly positioned within side locating pockets 1107 in transition portion 1106 of ultrasound probe 1100.

As shown in FIG. 11E-11H, rear securing member 1120 includes a lock assembly hinge 1121 for pivotably connecting to lock assembly 1114 and a latch engaging feature 1123 for releasably engaging a latch component 1138, as described below. In one exemplary implementation, lock assembly hinge 1121 projects upwardly from an upper portion of one side of rear securing member 1120 at a position laterally offset from guide tube 1116. As shown, lock assembly hinge 1121 includes a hinge pin 1125 for engagement with lock assembly 1114. Similarly, latch engaging feature 1123 also projects upwardly from an upper portion of one side of rear securing member 1120 at a position laterally offset from guide tube 1116 opposite from lock assembly hinge 1121. As shown in FIG. 11H, latch engaging feature 1123 includes an aperture 1127 for engaging latch component 1138, as described below.

FIGS. 11I-11L are top, left side, bottom, and exploded isometric views, respectively, of lock assembly 1114 consistent with implementations described herein. As shown, lock assembly 1114 includes a lever component 1136 and a latch component 1138 which together cooperatively engage guide tube 1116 and ultrasound probe 1100 to secure needle guidance device 1100 to ultrasound probe 1100 in the manner described below.

As shown in FIGS. 11I-11K, lever component 1136 includes a body portion 1140 having a pair of opposing arms 1142a and 1142b and a guide tube alignment portion 1144. Arms 1142a and 1142b are sized to accommodate at least a portion of guide tube 1116 therebetween, as shown in FIG. 11A. In one implementation, an intermediate portion of arm 1142a includes an engagement portion 1146 for pivotally coupling to lock assembly hinge 1121. As shown in FIGS. 11I and 11K, engagement portion 1146 projects outwardly relative to arm 1142a and includes one or more apertures 1148 therethrough for aligning with hinge pin 1125. During assembly, apertures 1148 are coupled to hinge pin 1125, such that lever component 1136 is able to pivot into and out of engagement with guide tube 1116.

As shown in FIGS. 11I and 11K, an intermediate portion of arms 1142a and 1142b include locating pocket engagement elements 1160 which project downwardly therefrom. As shown in FIG. 11A, when locked onto ultrasound probe 1100, pocket engagement elements 1160 engage upper locating pockets 1108 in transition portion 1106 of ultrasound probe 1100. When pocket engagement elements 1160 engage upper locating pockets 1108 and locating elements 1134 are slidingly positioned within side locating pockets 1107, needle guidance device becomes fixed relative to ultrasound probe 1100.

As shown, a rearward end of lever component 1136 includes a guide tube alignment portion 1144 which joins arms 1142. As shown in FIG. 11K, guide tube alignment portion 1144 includes an upper guide tube engagement feature 1164 and a central aperture 1166 longitudinally positioned to align with central aperture 1122 in guide tube 1116, when latch component 1138 is engaged with lever component 1136 and guide tube 1116, as described below. As shown in FIG. 11B, upper guide tube engagement feature 1164 includes a portion which projects forwardly relative to a remainder of guide tube alignment portion 1144. When lock assembly 1114 is latched onto guide tube 1116, upper guide tube engagement feature 1164 is configured to engage an upper, rear end of guide tube 1116 in a position in which aperture 1166 is aligned with aperture 1122.

As shown in FIGS. 11A and 111-K, guide tube alignment portion 1144 further includes an outwardly projecting latch tab 1168. As shown, latch tab 1168 include an aperture 1170 formed therethrough to engage latch component 1138, as described below. In one exemplary implementation, apertures 1170 may include a threaded configuration for engaging a threaded portion of latch component 1138.

As shown in FIGS. 11I-11L and FIG. 11A-11B, latch component 1138 comprises a rod 1172 and a latching knob 1174. As shown, during use, rod 1172 is configured to threadingly engage both latch tab 1168 and latch engaging feature 1123 in rear securing member 1120. In particular, as shown, rod 1172 is sized for receipt within apertures 1170 and 1127 and may include a threaded portion for engaging a correspondingly threaded portion of aperture 1170. When fully inserted or threaded into aperture 1170, as shown in FIG. 11A, a forward portion of rod 1172 engages aperture 1127 in latch engaging feature 1123 to secure latch component 1138 to guide tube 1116.

To secure needle guidance device 1110 to ultrasound probe 1100, an operator positions guide tube 1116 relative to ultrasound probe 1100 such that forward securing member 1118 and rearward securing member 1120 of guide tube 1116 engage forward end 1102 and transition portion 1106 of ultrasound probe 1100, respectively. Guide tube 1116 is then fixed relative to ultrasound probe 1100 by seating locating elements 1134 within side locating pockets 1107.

Lever component 1136 is then pivoted about hinge pin 1125 such that upper pocket engaging elements 1160 become seated within upper locating pockets 1108. Rod 1172 is inserted through apertures 1170, such as by turning knob 1174. In this manner, the forward end of rod 1172 is brought into engagement with aperture 1127 in latch engaging feature 1123, effectively securing guide tube 1116 to ultrasound probe 1100.

Consistent with embodiments described herein, central aperture 1166 in guide tube alignment portion 1144 is only aligned with central aperture 1122 in guide tube 1116 when guide tube 1116 is securely latched onto ultrasound probe, thus preventing premature or unsafe use of needle guidance device 1110.

Upon completion of a procedure, needle guidance device 1110 may be removed from ultrasound probe 1100 by reversing the above steps. That is, rod 1172 may be moved (e.g., threadingly) rearwardly to release latch engaging feature 1123 from the forward end of rod 1172. Lever component 1136 may then be pivoted about hinge pin 1125 such that upper pocket engagement elements 1160 become disengaged from upper locating pockets 1108. At this point, needle guidance device 1110 may be removed from ultrasound probe 1100 by sliding guide body 1112 forward relative to ultrasound probe 1100.

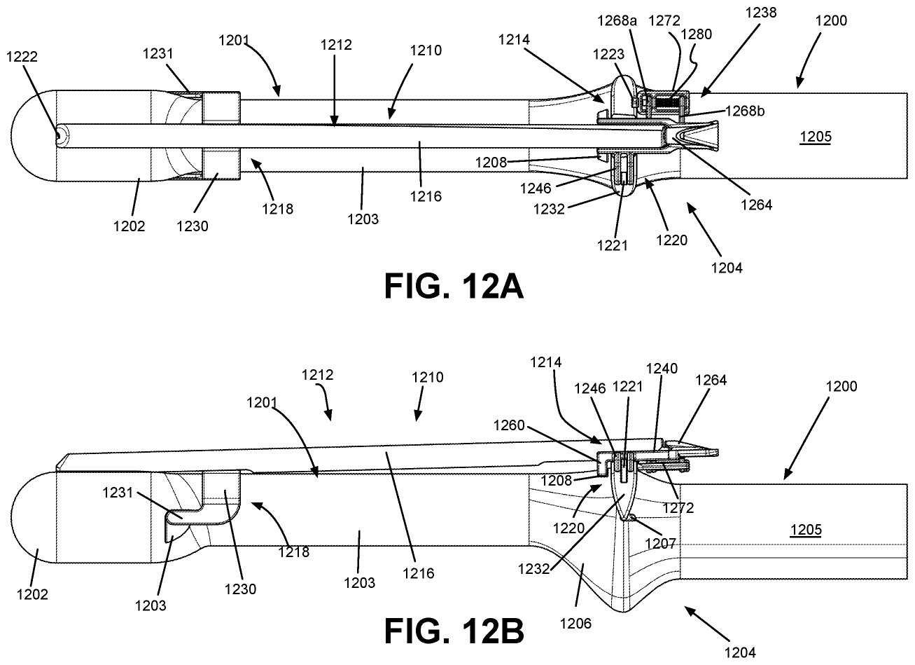

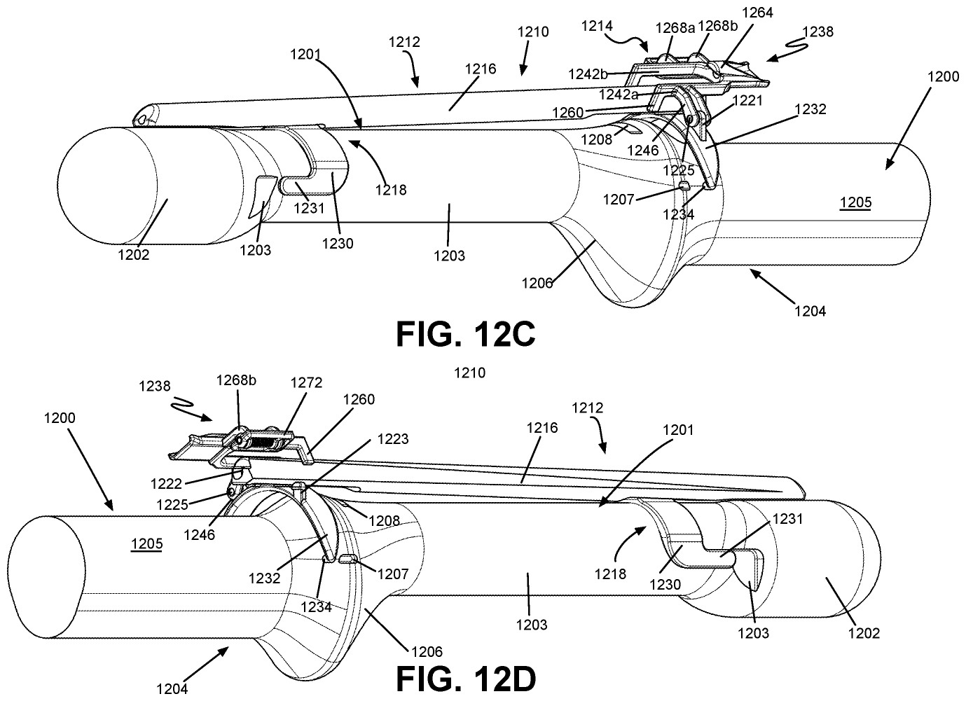

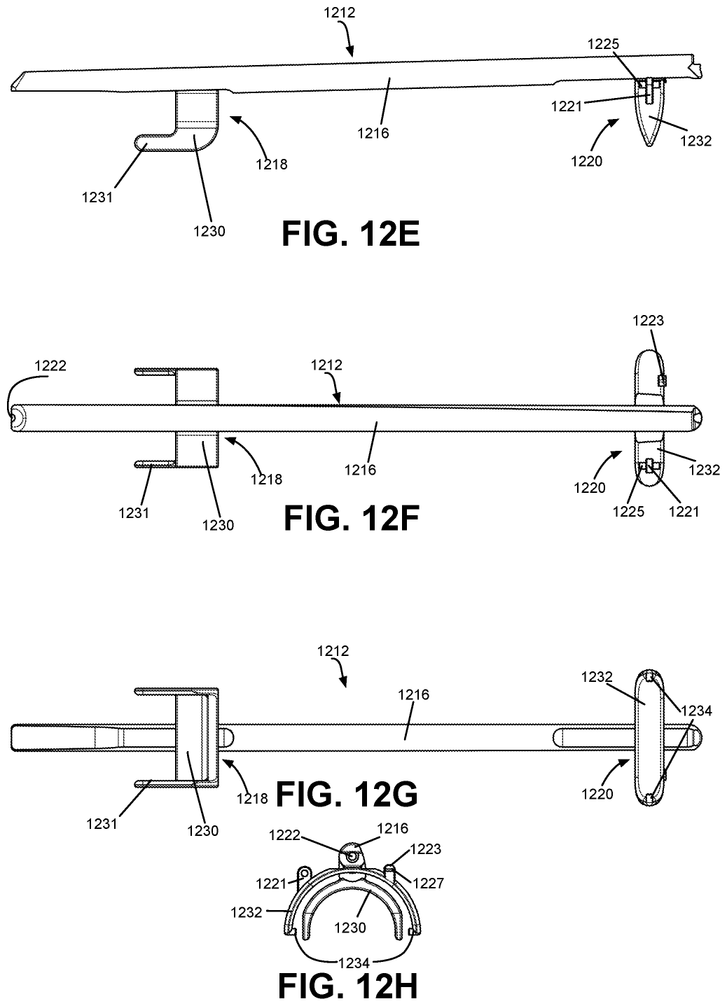

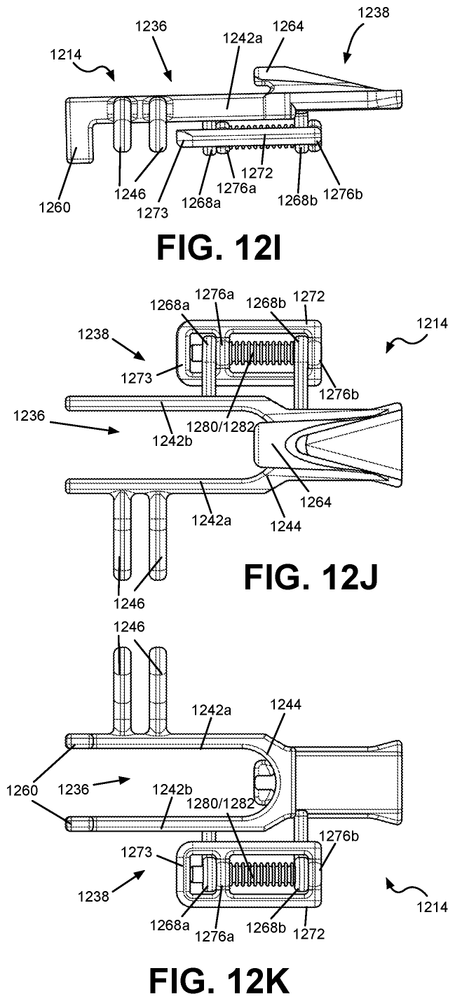

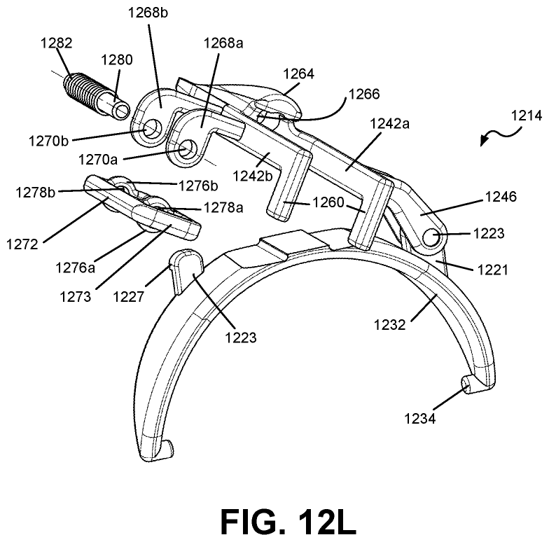

FIGS. 12A and 12B are top and left side views, respectively, illustrating one embodiment of a needle guidance device 1210 for use with an ultrasound probe 1200 in an assembled or locked configuration, consistent with embodiments described herein. FIG. 12C and 12D are left side plan and right rear isometric views, respectively, illustrating needle guidance device 1210 and ultrasound probe 1200 in an open or pre-assembled configuration.

Consistent with embodiments described herein, ultrasound probe 1200 and needle guidance device 1210 are configured to be lockingly coupled to each other so that, during use, needle guidance device 1210 is fixed relative to ultrasound probe 1200. As shown in FIGS. 12A-12D ultrasound probe 1200 includes one or more features that facilitate cooperative engagement with needle guidance device 1210. In particular, ultrasound probe 1200 includes a generally tubular body 1201 having a forward end 1202 and a rearward end 1204.

As shown, forward end 1202 includes a generally bulbous portion having a diameter larger than that of the intermediate portion between the forward and rearward ends. Consistent with embodiments described herein, a rearward portion of forward end 1202 includes a pair of forward locating pockets 1203 positioned on opposite sides of probe 1200. Forward locating pockets 1203 include a rearward-facing cavity having a forward wall therein. As described in additional detail below, forward locating pockets 1203 facilitate positioning and securing of needle guidance device 1210 to ultrasound probe 1200 during assembly.

Rearward end 1204 includes a handle portion 1205 and a transition portion 1206 to transition a shape of body 1201 between handle portion 1205 and the intermediate portion. As shown, transition portion 1206 includes a least a portion having a larger diameter than that of the intermediate portion and handle portion 1205.

Consistent with implementations described herein, transition portion 1206 includes a pair of locating pockets 1207 positioned on opposite sides of probe 1200 and a locating slot 1208 formed within the top surface of probe 1200. Locating pockets 1207 include a rearward-facing cavity having a front wall therein. As described in additional detail below, locating pockets 1207 and locating slot 1208 collectively facilitate positioning and securing of needle guidance device 1210 to ultrasound probe 1200 during assembly.

Needle guidance device 1210 includes a guide body 1212 and a lock assembly 1214. Guide body 1212 includes a guide tube 1216, a forward securing member 1218, and a rear securing member 1220. FIGS. 12E-12H are left side, top, bottom, and front views, respectively, of guide body 1212.

As shown in FIGS. 12E-12H, guide tube 1216 comprises a generally cylindrical configuration having a central aperture 1222 extending therethrough and sized for receiving a needle or other puncture device. In some implementations, guide tubes 1216 having different sized apertures 1222 may be provided for different gauge puncture devices. In some embodiments, rather than an enclosed central aperture 1222, guide tube 1216 may include a channel or slot formed vertically therein, into which the puncture device is received.

Forward securing member 1218 includes opposing curved arms 1230 that project downwardly from a forward portion of guide tube 1216 and which include a shape configured to correspond to a shape of forward end 1202 of ultrasound probe 1200. During assembly, curved arms 1230 are configured to engage forward end 1202 of ultrasound probe 1200 to positively center guide tube 1216 longitudinally with respect to ultrasound probe 1200. Consistent with the embodiment of FIGS. 12A-12H, curved arms 1230 terminate in forward pocket engaging members 1231 for engaging forward locating pockets 1203 in ultrasound probe 1200. In one implementation, forward pocket engaging members 1231 comprise a pair of arms that project forwardly and substantially perpendicularly from curved arms 1230. As shown, forward pocket engaging members 1231 are sized for receipt with forward locating pockets 1203.

In this manner, insertion of forward end 1202 of ultrasound probe 1200 within curved arms 1230 effectively captures an axial position of the forward end of guide tube 1216 relative to ultrasound probe 1200. That is, the forward end of guide tube 1216 may not be pulled radially outwardly from ultrasound probe 1200 when forward end 1202 of ultrasound probe 1200 is positioned with curved arms 1230.

Rear securing member 1220 includes curved arms 1232 that project downwardly from guide tube 1216 and which are configured to engage an outer surface of transition portion 1206 of ultrasound probe 1200. Consistent with some implementations described herein, curved arms 1232 in rear securing member 1220 include locating elements 1234 which project inwardly at a terminus of curved arms 1232. As shown in FIG. 12A, when assembled, locating elements 1234 are slidingly positioned within locating pockets 1207 in transition portion 1206 of ultrasound probe 1200.

As shown in FIGS. 12E-12H, rear securing member 1220 includes a lock assembly hinge 1221 for pivotably connecting to lock assembly 1214 and a latch engaging feature 1223 for releasably engaging a latch component 1238, as described below. In one exemplary implementation, lock assembly hinge 1221 projects upwardly from an upper portion of one side of rear securing member 1220 at a position laterally offset from guide tube 1216. As shown, lock assembly hinge 1221 includes a hinge pin 1225 for engagement with lock assembly 1214. Similarly, latch engaging feature 1223 also projects upwardly from an upper portion of one side of rear securing member 1220 at a position laterally offset from guide tube 1216 opposite from lock assembly hinge 1221. As shown in FIG. 12H, latch engaging feature 1223 includes a shoulder portion 1227 for engaging latch component 1238 when in the locked or closed position, as described below.

FIGS. 12H-12K are left side, top, bottom, and exploded isometric views, respectively, of lock assembly 1214 consistent with implementations described herein. As shown, lock assembly 1214 includes a lever component 1236 and a latch component 1238 which together cooperatively engage guide tube 1216 and ultrasound probe 1200 to secure needle guidance device 1200 to ultrasound probe 1200 in the manner described below.

As shown in FIGS. 12I-12K, lever component 1236 includes a body portion 1240 having a pair of opposing arms 1242a and 1242b and a guide tube alignment portion 1244. Arms 1242a and 1242b are sized to accommodate at least a portion of guide tube 1216 therebetween, as shown in FIG. 12A. In one implementation, an intermediate portion of arm 1242a includes an engagement portion 1246 for pivotally coupling to lock assembly engagement feature 1221. As shown in FIGS. 12H and 12K, engagement portion 1246 projects outwardly relative to arm 1242a and includes one or more apertures 1248 therethrough for aligning with hinge pin 1223. During assembly, apertures 1248 are coupled to hinge pin 1223, such that lever component 1236 is able to pivot into and out of engagement with guide tube 1216.

As shown in FIGS. 12I and 12K, arms 1242 a forward portion of arms 1242a and 1242b include upper pocket engagement elements 1260 which project downwardly therefrom. As shown in FIG. 12A, when locked onto ultrasound probe 1200, upper pocket engagement elements 1260 engage upper locating pockets 1208. When upper pocket engagement elements 1260 engage upper pockets 1208 and locating elements 1234 are slidingly positioned within side locating pockets 1207, needle guidance device becomes fixed relative to ultrasound probe 1200. As shown in FIG. 12D, in some implementations, at least a portion of guide tube 1216 may be cut away to accommodate arm 1242b and its associated upper pocket engagement element 1260.

As shown, a rearward end of lever component 1236 includes a guide tube alignment portion 1244 which joins arms 1242a/1242b. As shown in FIG. 12K, guide tube alignment portion 1244 includes an upper guide tube engagement feature 1264 and a central aperture 1266 longitudinally positioned to align with central aperture 1222 in guide tube 1216, when latch component 1238 is engaged with lever component 1236 and guide tube 1216, as described below. As shown in FIG. 12B, upper guide tube engagement feature 1264 includes a portion which projects forwardly relative to a remainder of guide tube alignment portion 1244. When lock assembly 1214 is latched onto guide tube 1216, upper guide tube engagement feature 1264 is configured to engage an upper, rear end of guide tube 1216 in a position in which aperture 1266 is aligned with aperture 1222.

As shown in FIGS. 12A and 12I-12K, guide tube alignment portion 1244 further includes a pair of outwardly projecting latch tabs 1268a and 1268b. Latch tabs 1268a and 1268b include apertures 1270a and 1270b formed therethrough and function to engage latch component 1238, as described below.

As shown in FIGS. 12I-12L and FIGS. 12A-12B, latch component 1238 comprises a generally frame-like member 1272 configured to engage shoulder portion 1227 in latch engaging feature 1223. In particular, as shown, latch component 1238 includes a latch engagement portion 1273 and a pair of latch tab engagement elements 1276a and 1276b that include alignment apertures 1278a and 1278b formed therethrough. Latch engagement portion 1273 projects forwardlly relative to forward tab engagement element 1276a and engages a shoulder portion 1227 when latch component 1238 is in a latched position with respect to guide tube 1216.

During assembly, latch tabs 1268a and 1268b are inserted within latch component 1238 such that tab engagement elements 1276a and 1276b abut respective rearward surfaces of latch tabs 1268a and 1268b. A rod 1280 having a biasing element, such as a spring 1282 is positioned thereon is inserted and secured within apertures 1270a/1270b and 1278a/1278b, such that biasing element 1282 is positioned between a rearward side of forward tab engagement element 1276 and a forward side of rearward tab 1268b, as shown in FIG. 121.

To secure needle guidance device 1210 to ultrasound probe 1200, an operator positions guide tube 1216 relative to ultrasound probe 1200 such that forward securing member 1218 and rearward securing member 1220 of guide tube 1216 engage forward end 1202 and transition portion 1206 of ultrasound probe 1200, respectively. Guide tube 1216 is then fixed relative to ultrasound probe 1200 by seating forward pocket engaging members 1231 in forward locating pockets 1203 and locating elements 1234 within locating pockets 1207.

Latch component 1238 is then opened by pulling rearwardly on frame-like member 1272, thus compressing biasing element 1282 and allowing latch engagement portion 1273 to move rearwardly relative to latch tabs 1268a and 1268b. Latch component 1238 is then pivoted downward about hinge pin 1225 such that slot engagement elements 1268 engage locating slot 1208. Frame-like member 1272 is then released, which causes biasing element 1282 to urge latch engagement portion 1273 to move forwardly relative to latch engaging feature 1223. In its forward position, latch engagement portion 1273 engages shoulder portion 1227, effectively securing guide tube 1216 to ultrasound probe 1200.

Consistent with embodiments described herein, central aperture 1266 in guide tube alignment portion 1244 is only aligned with central aperture 1222 in guide tube 1216 when guide tube 1216 is securely latched onto ultrasound probe, thus preventing premature or unsafe use of needle guidance device 1210.

Upon completion of a procedure, needle guidance device 1210 may be removed from ultrasound probe 1200 by reversing the above steps. That is, latch engagement portion 1273 may be again moved rearwardly to release guide tube 1216 from lower guide tube engagement feature 1274. Lever component 1236 may then be pivoted about hinge pin 1225 such that slot engagement elements 1268 becomes disengaged from locating slot 1208. At this point, needle guidance device 1210 may be removed from ultrasound probe 1200 by removing locating elements 1234 from locating pockets 1207.

The foregoing description of exemplary implementations provides illustration and description but is not intended to be exhaustive or to limit the embodiments described herein to the precise form disclosed. Modifications and variations are possible in light of the above teachings or may be acquired from practice of the embodiments.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements.

What is claimed is:

1. A puncture device guide, comprising:
a guide body member configured to fixedly attach to an ultrasound probe; and
a lock assembly coupled to the guide body to fix the guide body relative to the ultrasound probe,
wherein the guide body member comprises:
a guide tube having a central aperture extending therethrough for receiving a puncture device;
a forward securing member for engaging a forward portion of the ultrasound probe; and
a rearward securing member for engaging a rearward portion of the ultrasound probe, and
wherein the lock assembly is configured to be moveably coupled to the guide body member between a locked configuration and an unlocked configuration.

2. The puncture device guide of claim 1, wherein at least one of the forward securing member and the rearward securing member are configured to receive the ultrasound probe axially in one direction,
wherein at least one of the ultrasound probe or the rearward securing member include a structure which prevents continued axial advancement of ultrasound probe beyond a predefined location.

3. The puncture device guide of claim 2, wherein the rearward securing member comprises a pair of curved arms projecting downwardly from the guide tube,
wherein the pair of curved arms are configured to engage a transition portion of the ultrasound probe.

4. The puncture device of claim 3, wherein each of the pair of curved arms comprises a pocket engagement element for engaging a respective locating pocket in the transition portion of the ultrasound probe.

5. The puncture device guide of claim 2, wherein the rearward securing member comprises a ring configuration projecting downwardly from the guide tube.

6. The puncture device guide of claim 1, wherein the lock assembly comprises:
a lever component pivotably coupled to the guide body; and
a latch component movably coupled to the lever component.

7. The puncture device of claim 6,
wherein the lever portion comprises:
a pair of opposing arms configured to receive the guide tube therebetween; and
an upper guide tube engagement feature for engaging an upper portion of the guide tube when in the locked configuration,
wherein the opposing arms are pivotally coupled to the guide tube via a pivot pin.

8. The puncture device guide of claim 7, wherein the lever portion further comprises an upper locating engagement feature for engaging an upper locating feature on the ultrasound probe when in the locked configuration.

9. The puncture device guide of claim 8, wherein the ultrasound probe comprises a locating rib on an upper surface of the transition portion, and wherein the upper locating engagement feature comprises a rib engagement element for engaging the locating rib on the ultrasound probe when in the locked configuration.

10. The puncture device guide of claim 9 wherein the rib engagement element and the locating rib are located forward of rearward securing member.

11. The puncture device guide of claim 9 wherein the rib engagement element and the locating rib are located rearward of rearward securing member.

12. The puncture device guide of claim 7, wherein the lever portion further comprises a pair of upper locating engagement features for engaging the rearward securing member when in the locked configuration.

13. The puncture device guide of claim 7,
wherein the rear securing member comprises a lock assembly engagement feature projecting upwardly therefrom,
wherein the lever component comprises an engagement portion projecting outwardly therefrom, and
wherein the engagement portion of the lever component is configured to pivotably couple to the lock assembly engagement feature of the rear securing member to allow the lever component to move between the locked and unlocked configurations.

14. The puncture device guide of claim 7, wherein latch component comprises:
a lower guide tube engagement feature for engaging a lower portion of the guide tube when in the locked configuration,
wherein the lower guide tube engagement feature is movable into an out of engagement with the lower portion of the guide tube to allow movement between the locked and unlocked configurations.

15. The puncture device guide of claim 14, wherein the latch component comprises a biasing element which biases the lower guide tube engagement feature into engagement with the lower portion of the guide tube.

16. The puncture device guide of claim 14, wherein the lower guide tube engagement feature comprises a threaded rod.

17. The puncture device guide of claim 7, wherein the rearward securing member comprises a latch engaging feature; and
wherein latch component comprises:
a latch engaging portion for engaging a latch engaging feature of the rearward securing member when in the locked configuration,
wherein the latch engaging portion is movable into an out of engagement with the latch engaging feature of the rearward securing member to allow movement between the locked and unlocked configurations.

18. The puncture device guide of claim 17, wherein the latch component comprises a biasing element which biases the latch engaging portion into engagement with the latch engaging feature of the rearward securing member.

19. The puncture device guide of claim 17, wherein the latch engaging portion of the latch component comprises a threaded rod.
